SEQUENCE LISTING (253 sequences)

SEQ ID NO: 1 - RNA, 1065 nt, Homo sapiens
  source 1..1065: mol_type = genomic RNA
atgctgtttaatctgaggatcctgttaaacaatgcagcttttagaaatggtcacaacttcatggttcgaaattttcggtgtggacaaccactacaaaataaagtgcagctgaagggccgtgaccttctcactctaaaaaactttaccggagaagaaattaaatatatgctatggctatcagcagatctgaaatttaggataaaacagaaaggagagtatttgcctttattgcaagggaagtccttaggcatgatttttgagaaaagaagtactcgaacaagattgtctacagaaacaggctttgcacttctgggaggacatccttgttttcttaccacacaagatattcatttgggtgtgaatgaaagtctcacggacacggcccgtgtattgtctagcatggcagatgcagtattggctcgagtgtataaacaatcagatttggacaccctggctaaagaagcatccatcccaattatcaatgggctgtcagatttgtaccatcctatccagatcctggctgattacctcacgctccaggaacactatagctctctgaaaggtcttaccctcagctggatcggggatgggaacaatatcctgcactccatcatgatgagcgcagcgaaattcggaatgcaccttcaggcagctactccaaagggttatgagccggatgctagtgtaaccaagttggcagagcagtatgccaaagagaatggtaccaagctgttgctgacaaatgatccattggaagcagcgcatggaggcaatgtattaattacagacacttggataagcatgggacaagaagaggagaagaaaaagcggctccaggctttccaaggttaccaggttacaatgaagactgctaaagttgctgcctctgactggacatttttacactgcttgcccagaaagccagaagaagtggatgatgaagtcttttattctcctcgatcactagtgttcccagaggcagaaaacagaaagtggacaatcatggctgtcatggtgtccctgctgacagattactcacctcagctccagaagcctaaattttga

SEQ ID NO: 2 - DNA, 1065 bp, Homo sapiens
  source 1..1065: mol_type = genomic DNA
atgctgtttaatctgaggatcctgttaaacaatgcagcttttagaaatggtcacaacttcatggttcgaaattttcggtgtggacaaccactacaaaataaagtgcagctgaagggccgtgaccttctcactctaaaaaactttaccggagaagaaattaaatatatgctatggctatcagcagatctgaaatttaggataaaacagaaaggagagtatttgcctttattgcaagggaagtccttaggcatgatttttgagaaaagaagtactcgaacaagattgtctacagaaacaggctttgcacttctgggaggacatccttgttttcttaccacacaagatattcatttgggtgtgaatgaaagtctcacggacacggcccgtgtattgtctagcatggcagatgcagtattggctcgagtgtataaacaatcagatttggacaccctggctaaagaagcatccatcccaattatcaatgggctgtcagatttgtaccatcctatccagatcctggctgattacctcacgctccaggaacactatagctctctgaaaggtcttaccctcagctggatcggggatgggaacaatatcctgcactccatcatgatgagcgcagcgaaattcggaatgcaccttcaggcagctactccaaagggttatgagccggatgctagtgtaaccaagttggcagagcagtatgccaaagagaatggtaccaagctgttgctgacaaatgatccattggaagcagcgcatggaggcaatgtattaattacagacacttggataagcatgggacaagaagaggagaagaaaaagcggctccaggctttccaaggttaccaggttacaatgaagactgctaaagttgctgcctctgactggacatttttacactgcttgcccagaaagccagaagaagtggatgatgaagtcttttattctcctcgatcactagtgttcccagaggcagaaaacagaaagtggacaatcatggctgtcatggtgtccctgctgacagattactcacctcagctccagaagcctaaattttga

SEQ ID NO: 3 - protein, 353 aa, Homo sapiens
MLFNLRILLNNAAFRNGHNFMVRNFRCGQPLQNKVQLKGRDLLTLKNFTGEEIKYMLWLSADLKFRIKQKGEYLPLLQGKSLGMIFEKRSTRTRLSTETGFALLGGHPCFLTTQDIHLGVNESLTDTARVLSSMADAVLARVYKQSDLDTLAKEASIPIFGLSDLYHPIQILADYLTLQEHYSSLKGLTLSWIGDGNNILHSIMMSAAKFGMHLQAATPKGYEPDASVTKLAEQYAKENGTKLLLTNDPLEAAHGGNVLITDTWISMGQEEEKKKRLQAFQGYQVTMKTAKVAASDWTFLHCLPRKPEEVDDEVFYSPRSLVFPEAENRKWTIMAVMVSLLTDYSPQLQKPKF

SEQ ID NO: 4 - protein, 355 aa, synthetic construct
  REGION 1..355: note = Synthetic construct
MLVFNLRILLNNAAFRNGHNFMVRNFRCGQPLQNRVQLKGRDLLTLKNFTGEEIRYMLWLSADLKFRIKQKGEYLPLLQGKSLGMIFEKRSTRTRLSTETGFALLGGHPCFLTTQDIHLGVNESLTDTARVLSSMADAVLARVYKQSDLDTLAKEASIPIINGLSDLYHPIQILADYLTLQEHYSSLKGLTLSWIGDGNNILHSIMMSAAKFGMHLQAATPKGYEPDASVTKLAEQYAKENGTKLLLTNDPLEAAHGGNVLITDTWISMGQEEEKKKRLQAFQGYQVTMKTAKVAASDWTFLHCLPRKPEEVDDEVFYSPRSLVFPEAENRKWTIMAVMVSLLTDYSPQLQKPKF

SEQ ID NO: 5 - RNA, 129 nt, synthetic construct
  misc_feature 1..129: note = Synthetic construct
  source 1..129: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatag

SEQ ID NO: 6 - RNA, 28 nt, synthetic construct
  misc_feature 1..28: note = Synthetic construct
  source 1..28: mol_type = other RNA
attattacatcaaaacaaaaagccgcca

SEQ ID NO: 7 - RNA, 245 nt, synthetic construct
  misc_feature 1..245: note = Synthetic construct
  source 1..245: mol_type = other RNA
cttaagggggcgctgcctacggaggtggcagccatctccttctcggcatcaagcttaccatggtgccccaggccctgctcttggtcccgctgctggtgttccccctctgcttcggcaagttccccatctacaccatccccgacaagctggggccgtggagccccatcgacatccaccacctgtcctgccccaacaacctcgtggtcgaggacgagggctgcaccaacctgagcgggttctcctac

SEQ ID NO: 8 - RNA, 177 nt, synthetic construct
  misc_feature 1..177: note = Synthetic construct
  source 1..177: mol_type = other RNA
tgagtgtcgtacagcctccaggcccccccctcccgggagagccatagtggtctgcggaaccggtgagtacaccggaattgccgggaagactgggtcctttcttggataaacccactctatgcccggccatttgggcgtgcccccgcaagactgctagccgagtagtgttgggttgcg

SEQ ID NO: 9 - RNA, 89 nt, synthetic construct
  misc_feature 1..89: note = Synthetic construct
  source 1..89: mol_type = other RNA
aattattggttaaagaagtatattagtgctaatttccctccgtttgtcctagcttttctcttctgtcaaccccacacgcctttggcaca

SEQ ID NO: 10 - RNA, 569 nt, synthetic construct
  misc_feature 1..569: note = Synthetic construct
  source 1..569: mol_type = other RNA
ctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacgtgtgtttagtcgaggttaaaaaacgtctaggccccccgaaccacggggacgtggttttcctttgaaaaacacgatgataat

SEQ ID NO: 11 - RNA, 44 nt, synthetic construct
  misc_feature 1..44: note = Synthetic construct
  source 1..44: mol_type = other RNA
gtcagctttcaaactctttgtttcttgtttgttgattgagaata

SEQ ID NO: 12 - RNA, 54 nt, synthetic construct
  misc_feature 1..54: note = Synthetic construct
  source 1..54: mol_type = other RNA
ctctcgcctgagaaaaaaaatccacgaaccaatttctcagcaaccagcagcacg

SEQ ID NO: 13 - RNA, 52 nt, synthetic construct
  misc_feature 1..52: note = Synthetic construct
  source 1..52: mol_type = other RNA
acctgtgagggttcgaaggaagtagcagtgttttttgttcctagaggaagag

SEQ ID NO: 14 - RNA, 71 nt, synthetic construct
  misc_feature 1..71: note = Synthetic construct
  source 1..71: mol_type = other RNA
acacagaaacattcgcaaaaacaaaatcccagtatcaaaattcttctctttttttcatatttcgcaaagac

SEQ ID NO: 15 - RNA, 52 nt, synthetic construct
  misc_feature 1..52: note = Synthetic construct
  source 1..52: mol_type = other RNA
cagaaaaatttgctacattgtttcacaaacttcaaatattattcatttattt

SEQ ID NO: 16 - RNA, 158 nt, synthetic construct
  misc_feature 1..158: note = Synthetic construct
  source 1..158: mol_type = other RNA
ctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacat

SEQ ID NO: 17 - RNA, 166 nt, synthetic construct
  misc_feature 1..166: note = Synthetic construct
  source 1..166: mol_type = other RNA
tgcaaggctggccggaagcccttgcctgaaagcaagatttcagcctggaagagggcaaagtggacgggagtggacaggagtggatgcgataagatgtggtttgaagctgatgggtgccagccctgcattgctgagtcaatcaataaagagctttcttttgacccat

SEQ ID NO: 18 - RNA, 143 nt, synthetic construct
  misc_feature 1..143: note = Synthetic construct
  source 1..143: mol_type = other RNA
acgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgca

SEQ ID NO: 19 - RNA, 220 nt, synthetic construct
  misc_feature 1..220: note = Synthetic construct
  source 1..220: mol_type = other RNA
tagagcggcaaaccctagctacactccatagctagtttcttttttttttgtttttttttttttttttttttttttttttttttttttttcctttcttttccttctttttttcctcttttcttggtggctccatcttagccctagtcacggctagctgtgaaaggtccgtgagccgcatgactgcagagagtgccgtaactggtctctctgcagatcatgt

SEQ ID NO: 20 - RNA, 170 nt, synthetic construct
  misc_feature 1..170: note = Synthetic construct
  source 1..170: mol_type = other RNA
acacatcacaaccacaaccttctcaggctaccctgagaaaaaaagacatgaagactcaggactcatcttttctgttggtgtaaaatcaacaccctaaggaacacaaatttctttaaacatttgacttcttgtctctgtgctgcaattaataaaaaatggaaagaatctac

SEQ ID NO: 21 - RNA, 110 nt, synthetic construct
  misc_feature 1..110: note = Synthetic construct
  source 1..110: mol_type = other RNA
gctggagcctcggtagccgttcctcctgcccgctgggcctcccaacgggccctcctcccctccttgcaccggcccttcctggtctttgaataaagtctgagtgggcagca

SEQ ID NO: 22 - RNA, 123 nt, synthetic construct
  misc_feature 1..123: note = Synthetic construct
  source 1..123: mol_type = other RNA
tagtgcagtcactggcacaacgcgttgcccggtaagccaatcgggtatacacggtcgtcatactgcagacagggttcttctactttgcaagatagtctagagtagtaaaataaatagtataag

SEQ ID NO: 23-25 - skipped (3 entries)

SEQ ID NO: 26 - RNA, 1368 nt, synthetic construct
  misc_feature 1..1368: note = Synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctgtttaatctgaggatcctgttaaacaatgcagcttttagaaatggtcacaacttcatggttcgaaattttcggtgtggacaaccactacaaaataaagtgcagctgaagggccgtgaccttctcactctaaaaaactttaccggagaagaaattaaatatatgctatggctatcagcagatctgaaatttaggataaaacagaaaggagagtatttgcctttattgcaagggaagtccttaggcatgatttttgagaaaagaagtactcgaacaagattgtctacagaaacaggctttgcacttctgggaggacatccttgttttcttaccacacaagatattcatttgggtgtgaatgaaagtctcacggacacggcccgtgtattgtctagcatggcagatgcagtattggctcgagtgtataaacaatcagatttggacaccctggctaaagaagcatccatcccaattatcaatgggctgtcagatttgtaccatcctatccagatcctggctgattacctcacgctccaggaacactatagctctctgaaaggtcttaccctcagctggatcggggatgggaacaatatcctgcactccatcatgatgagcgcagcgaaattcggaatgcaccttcaggcagctactccaaagggttatgagccggatgctagtgtaaccaagttggcagagcagtatgccaaagagaatggtaccaagctgttgctgacaaatgatccattggaagcagcgcatggaggcaatgtattaattacagacacttggataagcatgggacaagaagaggagaagaaaaagcggctccaggctttccaaggttaccaggttacaatgaagactgctaaagttgctgcctctgactggacatttttacactgcttgcccagaaagccagaagaagtggatgatgaagtcttttattctcctcgatcactagtgttcccagaggcagaaaacagaaagtggacaatcatggctgtcatggtgtccctgctgacagattactcacctcagctccagaagcctaaattttgactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 27 - RNA, 1368 nt, synthetic construct
  misc_feature 1..1368: note = Synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctctttaatctgcgcatcttactgaacaacgccgcattccggaacggtcacaacttcatggtccgcaatttccgctgtggccagccgcttcaaaacaaggtccagctgaagggacgggatctgctgacactgaagaacttcaccggagaagagatcaagtacatgctgtggctcagcgcagacttgaagttccggatcaagcagaagggagaatacttgcccctgctgcaaggaaagtcgctgggaatgatttttgagaagcggtcaactcgcaccagactctccaccgaaactggtttcgcactgcttggcgggcacccttgcttcctgacgactcaggacatccacctcggcgtgaacgaatcgctaaccgataccgccagagtgctttcttccatggccgacgcggtgctggccagggtgtacaagcagtccgacctcgataccttggcaaaggaggcttccattcccatcatcaacggcctgagcgacctgtaccacccaatccaaatcctggctgactacctgaccctgcaagagcactacagcagcctgaagggtctgaccctgtcatggattggcgatggaaacaatattctgcactccatcatgatgtccgccgcgaagttcggaatgcatctgcaagccgccactccaaaaggatacgaaccggatgcgtccgtgaccaagttggcggaacagtacgcgaaggagaacggaaccaagcttctgctgactaacgaccccctcgaggctgcgcatgggggcaacgtgctgattaccgacacctggatctccatggggcaggaggaagagaagaagaagagactgcaggcattccaggggtaccaggtcaccatgaaaaccgcaaaagtggcagcttcggactggactttcctgcattgcctgccgaggaagccggaggaagtcgacgacgaagtgttctactcgcctcggtccctggtgttccccgaggccgaaaaccggaagtggaccatcatggccgtgatggtgtccttgctgactgactatagcccgcagctgcagaagcctaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 28 - RNA, 1368 nt, synthetic construct
  misc_feature 1..1368: note = Synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctgtttaacctacgtattttgctcaacaatgcagcctttagaaacggacataactttatggttcgaaactttcgctgcgggcagccactgcagaacaaggtccagctgaaagggagagatttgctcacgctgaagaactttactggcgaagaaatcaagtatatgctgtggttgtccgcggacctcaagtttcggattaagcagaaaggggagtatctgccactgctgcaaggaaagagcctcggcatgatcttcgagaagcggagcactcggaccaggctgagtaccgaaactggcttcgcattgttgggtggacatccatgttttctgacaacgcaggacattcatctgggcgtgaacgagagtctgacggacacagctcgcgttctgtcctctatggctgatgcggtgttggcccgggtctataagcagtccgatttggacaccttggctaaggaagctagcataccgattatcaatgggctgtccgacctgtatcaccctattcaaatcctggccgactacctcacactgcaagaacactatagctcattgaagggactgaccctgagctggataggggacggaaacaacatcctacatagcattatgatgtccgctgccaagtttggcatgcatcttcaagccgccacgccaaagggttatgagcccgacgcgtcagtgacaaagctggccgagcagtacgctaaggagaatggtaccaaattactgctgactaatgatccactggaggctgcacatggcggcaatgtactgatcaccgacacatggatctcgatgggccaggaggaagaaaagaagaagaggcttcaggccttccaaggctaccaggtcaccatgaaaacagctaaggttgcagcatctgattggacctttctgcactgtctgccaaggaagcccgaagaggtggacgatgaagtattctatagcccacggagtttggtgttccctgaggctgaaaataggaagtggacaattatggccgtaatggtgtccctgttaaccgactactctccgcaactgcagaaacctaagttttagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 29 - RNA, 1368 nt, synthetic construct
  misc_feature 1..1368: note = Synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctgtttaacttaaggatcctgctgaacaacgccgcttttcgtaacggtcataactttatggtccggaactttagatgtggccagccgctgcagaacaaggttcagctgaaggggagggatctgctgaccttgaagaactttaccggcgaagagatcaagtacatgttgtggctgagcgccgatctgaagtttaggattaagcagaagggggagtatttgccactgctgcaaggaaaatccttggggatgatcttcgagaagcgctccactagaacccggctaagcacagaaaccggcttcgcacttctgggtggacatccctgttttctgacgacgcaggatatacacctgggcgtgaatgagagtctgacggacacagctagggtgttgagcagcatggccgatgcagtactggcccgcgtttataagcagagcgacttggacacactggccaaggaagcgtcaattccgattatcaatgggctgtcagacctgtatcatcccattcaaatcttggctgactatctgaccctgcaagaacattacagctccctgaagggcctcacgttgtcctggattggcgacggaaacaacattctgcattcgatcatgatgagcgctgctaagtttggcatgcacctccaagccgctacacctaagggatatgagcctgatgccagcgtaaccaagctggccgaacagtacgcgaaggagaatggcacgaaactgctgttgacaaatgacccactggaggcagctcacggtggcaacgtgctgatcaccgacacgtggatatctatgggacaggaagaagagaagaagaagcggctgcaggcattccaagggtatcaggtcaccatgaaaacggccaaggttgctgcatccgactggacatttctgcattgcttgccccgcaaaccagaagaagtagacgacgaagtcttttattccccacggtcgctggtgttccccgaggcggagaatcgaaagtggacgattatggccgtgatggtgtccctgctgactgattactctccccaactgcaaaagcctaagttttagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 30 - RNA, 1368 nt, synthetic construct
  misc_feature 1..1368: note = Synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgcttttcaacctgaggatcctcctgaacaacgccgcctttcgcaatggtcacaactttatggtccggaacttcagatgcggccagccgctgcagaacaaggtccagctgaagggacgggatctgctgactctgaagaacttcaccggagaagagatcaagtacatgctgtggctgtcggccgacctgaagttcaggatcaagcagaagggagaatacctcccgctgctgcaaggaaagtccctgggcatgattttcgagaagcgctcgaccagaactcggttgtccaccgaaaccgggtttgcgctgctgggcggacatccttgcttcctgacgactcaggatattcacctgggagtgaacgagtcgctgaccgacaccgccagagtgctgagctcgatggccgacgccgtgttggcacgcgtgtacaagcagtccgatctggataccctggccaaagaagcttccatcccgatcattaacgggctgagcgacctctaccaccccattcaaatcctggccgactacctgactctgcaagaacactacagctcgctgaaggggttgactctgtcctggatcggcgacggaaacaacatcctgcactccatcatgatgtcggccgcaaagttcggcatgcatttgcaagccgccaccccaaagggctacgaaccagacgcgagcgtcaccaagctggccgaacagtacgcgaaggaaaatggtactaagctgctgctgaccaacgacccattggaagctgcccatggtggaaacgtgctgatcaccgacacctggatctcgatgggccaggaagaggagaagaagaagcggctgcaggcgttccaggggtatcaggtcaccatgaaaacagccaaagtggcagcgtcagactggaccttcctccactgtctgcctcgcaagccagaggaggtggacgacgaggtgttctactcccctcggtccctcgtgttccctgaggctgagaaccggaagtggaccattatggccgtgatggtgtcactcctgactgattactccccgcaactgcagaagcccaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 31 - RNA, 1368 nt, synthetic construct
  misc_feature 1..1368: note = Synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctgtttaacctgaggatcctattgaacaatgctgcttttcgtaatggccataactttatggttcggaactttagatgcgggcagccactgcagaacaaggtccagttgaaaggccgcgatctgttgacattgaagaactttaccggcgaagagattaagtatatgctgtggctgtctgctgacctcaagtttcgaatcaagcagaagggcgaatatctccccctgctgcaaggaaagtctctcggcatgatctttgagaagcggagtacccgaacacggctgagcaccgaaacgggcttcgcactgctggggggccatccctgttttctgacaacgcaggacatccacttgggggttaacgaatcattgactgataccgcccgcgtactgtcatccatggccgacgctgtgctggctagggtgtacaagcagtcagatctggatacactggccaaggaagctagcataccaatcatcaatggactgagtgacctttatcacccgattcaaatactagccgattatctgaccctgcaagagcattactcctcgctgaaaggcctcacgctgtcctggatcggcgacggcaacaacattctgcatagtattatgatgtctgctgccaaattcggcatgcatctgcaagctgctacgccgaagggttatgaacccgacgcgtcagttacgaagctcgctgagcagtatgcaaaggagaatggcacaaagctgttgcttaccaacgatcccctggaagctgctcatggcggcaatgtgctgattactgacacctggatttcaatgggccaggaggaggagaagaagaagaggttacaggcttttcaaggttaccaagtcacgatgaaaaccgctaaggtcgcagccagcgactggacattcctgcactgtctgccaagaaagccggaagaagtggacgacgaggtgttctattccccgcggtctttggtgtttccggaggccgaaaacaggaaatggaccattatggccgtgatggtatcgttgctgacggactacagccctcagttgcaaaagcccaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 32 - RNA, 1368 nt, synthetic construct
  misc_feature 1..1368: note = Synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctctttaacctccgcatcctcctcaacaacgccgccttccggaatgggcataacttcatggtccggaacttcagatgcggccagcccctgcaaaacaaggtccagttgaagggacgggacctccttacgctgaagaactttaccggagaagagattaagtacatgctgtggttgtccgctgacctcaagttccgcattaagcagaagggagaatatctgccgctgctgcaaggaaagagcctgggcatgatcttcgaaaagcgctccactagaacccggctgtcgactgagactggattcgccttgctcggtggacacccgtgcttcctgacgacccaggacatccacctgggagtgaacgagtcacttacggataccgcgagggtgctgtcctcaatggccgacgcagtgctcgcgcgcgtgtacaagcagtcagatctggataccctggccaaggaagccagcattcccatcatcaacggactgagcgacctttaccacccaatccagatcctcgccgactacttaaccctgcaagagcactacagctccctgaagggactgactctgtcctggatcggggatggaaacaacatcctgcactccatcatgatgtctgccgctaagtttgggatgcatctgcaagccgcaacccctaagggatacgagcccgacgcctcggtgaccaaacttgcggaacagtacgccaaggaaaacggtaccaagctgctgctgaccaacgaccctctggaagcggcccacggaggaaatgtgctgattaccgacacctggatttcgatgggccaggaggaggagaagaagaagagactgcaggcgttccagggatatcaggtcaccatgaaaaccgccaaggtcgccgccagcgactggaccttcctgcactgtctccctcggaaaccggaagaagtggatgacgaggtgttctactccccgcgctcgctggtgttcccggaggctgaaaacaggaagtggacaatcatggccgtgatggtgtccctgttgaccgactactccccacaactgcagaagcccaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 33 - RNA, 1368 nt, synthetic construct
  misc_feature 1..1368: note = Synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgcttttcaatctgcgcatcctcctgaacaacgccgccttccgcaatggacacaactttatggtccgcaacttccgctgtgggcagccgctgcagaacaaggtccagctcaaggggagagatctcctgaccctgaagaacttcactggagaggagatcaagtacatgctgtggctgtccgccgacctgaaatttcggattaagcagaagggcgaatacctcccactgctgcaaggaaagtctttgggcatgatcttcgaaaagagaagcacccggacccggttgagcaccgaaactgggttcgcgctcctcggtggacacccgtgcttcctgaccacccaagatattcatctgggtgtcaacgaaagcctgaccgacaccgccagggtgctgtcatccatggctgacgcagtgctcgcccgggtgtacaagcagtcagacctggacaccctcgccaaggaagcttcgatccctatcatcaacggactttccgacctgtaccaccccatccaaattctggccgactacctgactctgcaagaacactatagctcgctgaaaggacttactctgtcctggatcggggacggcaacaacattctccattccatcatgatgtccgctgccaagttcggaatgcaccttcaagcagcgactcccaagggatacgaacctgatgcctccgtgactaagctggcagagcagtacgccaaggagaacggtacaaagctgctgctcacgaacgaccccctggaggcggcccacggcggaaacgtgctgattaccgatacctggatctcaatgggccaggaagaggagaagaagaagcggctccaggcgtttcaaggctaccaggtcaccatgaaaaccgcgaaggtcgccgcctccgactggactttcttgcactgcctgccgcggaagcccgaggaagtggatgacgaagtgttctactcgccgagatcgttggtgttccctgaggccgaaaacaggaagtggaccatcatggccgtgatggtgtccctgctgactgattacagcccacagctgcagaagcctaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 34 - RNA, 1368 nt, synthetic construct
  misc_feature 1..1368: note = Synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgcttttcaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 35 - RNA, 1368 nt, synthetic construct
  misc_feature 1..1368: note = Synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgcttttcaacctgagaatcctcctgaacaacgccgccttccgcaatggtcataacttcatggtccgcaactttcgctgcggacagcctctccaaaacaaggtccagctcaaggggcgcgacctcctcacactgaagaacttcactggagaagaaatcaagtacatgctgtggctgagcgccgatctgaagttccggatcaagcagaagggagagtaccttcctctgctgcaagggaagtccttgggaatgattttcgagaagcggtccacccggaccaggctgagcactgaaactggcttcgccctgctgggaggccacccttgtttcctgaccactcaggacatccacctgggcgtgaacgagtccctgaccgatactgccagagtgctgtcctccatggccgacgccgtgctcgcccgggtgtacaagcagtcagacctcgatacgctggccaaggaagcctccattcccattatcaatggtctgtcggacctctaccatccaatccaaatcctcgccgactacctgactctgcaagaacactacagctcactcaagggcctcaccctctcctggatcggcgacggaaacaacatccttcactcgattatgatgtcggccgcgaagttcgggatgcacctccaagctgccactccaaaaggctacgagccggatgcctcagtgactaagttggcggaacagtatgcgaaggagaacggtaccaagctcctgctgactaacgacccgctggaggccgcccacgggggaaacgtgctcatcaccgatacttggatttccatgggacaggaggaagagaagaagaagcggttgcaggcatttcagggctaccaggtcaccatgaaaactgccaaagtcgccgccagcgactggaccttcctgcactgcctgccccgcaagcctgaagaagtggacgacgaggtgttctactctccccggtccctcgtgttccctgaggccgaaaacaggaagtggaccatcatggctgtgatggtgtccctcctgaccgactacagccctcagctccaaaaacccaagttttagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 36 - RNA, 1368 nt, synthetic construct
  misc_feature 1..1368: note = Synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgcttttcaacctgagaatcctcttgaacaatgctgcttttcggaatggccacaactttatggttcggaacttccgttgcggccagcctttacaaaacaaggtccagctgaagggccgggatttgctcacactgaagaactttactggggaggagattaagtatatgctgtggctgtccgctgacctgaagtttaggatcaagcagaagggcgaatatctgccgctgctgcaagggaaaagtctgggcatgatttttgaaaagcgctctacccggaccagactgtctacggaaacaggctttgccctgctgggcggccacccctgttttctgacaacgcaggacatccatctgggcgtgaacgaatcactgaccgatactgctcgggtactcagttctatggctgacgcagtgctggctagggtgtacaagcagagcgacttggacacactggctaaggaggccagcatccccattatcaatggcctgtctgatttgtaccatcccattcaaatcctggctgattatctgacactacaagagcattactcaagtctgaagggtttgactctctcctggatcggcgacggcaacaacattttacattccattatgatgagtgctgctaagtttggcatgcatttgcaagctgctaccccaaagggctatgaacctgacgctagcgtaaccaagttggccgaacagtatgctaaagagaatggcaccaagctgctcctgacgaatgaccccctggaagctgctcatggcggaaacgtacttataactgatacatggattagcatgggccaggaagaggagaagaagaagagactgcaggccttccaaggctatcaggtcaccatgaaaactgccaaggttgcagctagcgactggaccttcctgcactgtttgccgaggaaacccgaggaggtggacgatgaagtcttttattctccccgctccttggtgtttcccgaggctgaaaatcgaaagtggacgataatggcagtgatggtgtccctactgaccgactattctccacaactgcagaagcctaaattctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 37 - RNA, 1368 nt, synthetic construct
  misc_feature 1..1368: note = Synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgcttttcaatctgaggatcctgctgaacaacgctgcttttcgcaacggtcataactttatggttcgcaattttcgttgtggccagccgctgcagaacaaggttcagctgaagggcagagatctgctgactctgaagaacttcactggggaagaaatcaagtatatgttatggctgtccgcggatctgaaatttcgaatcaagcagaagggcgaatatcttcccctgctgcaagggaaatccttgggcatgatttttgagaagaggagcactaggactagattgtcaacagaaacaggctttgctttgttgggcggacatccctgctttctgacgacacaggatatccacctcggcgtaaacgagtccctcaccgacactgctagggtactgagcagcatggccgacgctgtgctagcccgggtttacaagcagtcagacctggacacccttgccaaggaagcttctattccaattatcaacggcctgagtgacctgtatcaccctattcaaatactcgccgactatttgacgcttcaagaacattacagcagcctcaagggcttaaccttgagttggataggcgacggcaacaatatcctgcattccattatgatgtctgccgctaagtttggcatgcatctacaagccgcaacacccaagggctatgaacccgacgctagcgtgaccaagctggccgagcagtatgctaaggaaaatggcacaaagctccttcttaccaacgatcccctggaggctgctcacggcggcaacgtgctgattaccgatacatggattagcatgggccaggaggaggagaaaaagaagcggctccaggcttttcaaggctatcaggtcaccatgaaaactgcaaaggtcgctgcctccgactggactttcctgcattgtctaccccgcaagcctgaggaagtggacgatgaggtgttctactccccacggagtctggtgttcccggaagcagagaatcggaagtggaccatcatggctgtcatggtgtcgctcttgactgactattctccccaactgcaaaaacccaagttttagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 38 - RNA, 1368 nt, synthetic construct
  misc_feature 1..1368: note = Synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgttattcaaccttcgtatcctgctaaacaatgctgcttttcgcaatggccataactttatggttcgcaactttagatgcggccagccgctgcagaacaaggttcagctgaagggccgggacttgctgacgctgaaaaactttaccggggaagagattaagtatatgctgtggctaagcgctgatctgaagtttaggatcaagcagaagggcgaatatctgccactgctgcaagggaagagtcttggcatgatttttgaaaagcggtctaccagaacccggctgtcgaccgagacaggttttgctctgctggggggccatccctgttttctgacaactcaggacattcacctgggcgtgaatgagtccctgaccgatactgctagggtgttgagtagcatggccgacgctgtactcgctcgagtgtataagcagtctgatctggacactctggctaaggaagcttccattcctattatcaacggcttgagcgacctgtaccaccccattcaaatcctcgctgattacttgactttgcaagaacattacagcagcttgaagggcttaacactgagctggataggcgacggaaacaacatcttgcattccataatgatgtccgccgctaagttcgggatgcacctccaagcagccacacccaagggctatgaaccggatgcttccgtgacaaaactggctgagcagtatgctaaggagaatggcacgaaactgctgctcaccaacgacccattggaagctgcacatggtggcaacgtactgatcactgacacttggatctcaatgggccaggaggaagagaagaagaaaaggctgcaggcatttcagggataccaagtcactatgaaaactgccaaggtcgctgcctccgactggacattcctgcattgtctgccacggaagcctgaggaagtcgatgacgaagtgttctatagcccacgaagcttggtgtttcccgaggctgagaataggaagtggaccattatggctgttatggtgtccctgctcaccgactattcccctcaactgcaaaaacccaagttttagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 39 - RNA, 1365 nt, synthetic construct
  misc_feature 1..1365: note = Synthetic construct
  source 1..1365: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccatgctttttaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcccatcatcaacggcctgagcgacctgtaccacccaatccaaatcctggctgactacctgaccctgcaagagcactacagcagcctgaagggtctgaccctgtcatggattggcgatggaaacaatattctgcactccatcatgatgtccgccgcgaagttcggaatgcatctgcaagccgccacgccaaaaggatacgaaccggatgcgtccgtgacgaagttggcggaacagtacgcgaaggagaacggaaccaagcttctgctgactaacgaccccctcgaggctgcgcatgggggcaacgtgctgattaccgacacctggatctccatggggcaggaggaagagaagaagaagagactgcaggcattccaggggtaccaggtcaccatgaaaaccgcaaaagtggcagcttcggactggactttcctgcattgcctgccgaggaagccggaggaagtcgacgacgaagtgttctactcgcctcggtccctggtgttccccgaggccgaaaaccggaagtggaccatcatggccgtgatggtgtccttgctgactgactatagcccgcagctgcagaagcctaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 40 - RNA, 1365 nt, synthetic construct
  misc_feature 1..1365: note = Synthetic construct
  source 1..1365: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccatgcttttcaacctgagaatcctcttgaacaatgctgcttttcggaatggccacaactttatggttcggaacttccgttgcggccagcctttacaaaacaaggtccagctgaagggccgggatttgctcacactgaagaactttactggagaagagatcaagtacatgctgtggctgtcggccgacctgaagttcaggatcaagcagaagggagaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctcgataccttggcaaaggaggcttccattcccatcatcaacggcctgagcgacctgtaccacccaatccaaatcctggctgactacctgaccctgcaagagcactacagcagcctgaagggtctgaccctgtcatggattggcgatggaaacaatattctgcactccatcatgatgtccgccgcgaagttcggaatgcatctgcaagccgccactccaaaaggatacgaaccggatgcgtccgtgaccaagttggcggaacagtacgcgaaggagaacggaaccaagcttctgctgactaacgaccccctcgaggctgcgcatgggggcaacgtgctgattaccgacacctggatctccatggggcaggaggaagagaagaagaagagactgcaggcattccaggggtaccaggtcaccatgaaaaccgcaaaagtggcagcttcggactggactttcctgcattgcctgccgaggaagccggaggaagtcgacgacgaagtgttctactcgcctcggtccctggtgttccccgaggccgaaaaccggaagtggaccatcatggccgtgatggtgtccttgctgactgactatagcccgcagctgcagaagcctaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 41 - RNA, 1365 nt, synthetic construct
  misc_feature 1..1365: note = Synthetic construct
  source 1..1365: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccatgcttttcaacctgagaatcctcttgaacaatgctgcttttcggaatggccacaactttatggttcggaacttccgttgcggccagcctttacaaaacaaggtccagctgaagggccgggatttgctcacactaaagaactttactggagaagagatcaagtacatgctatggctgtcggccgacctgaagttccgtatcaagcagaagggagaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctcgataccttggcaaaggaggcttccattcccatcatcaacggcctgagcgacctgtaccacccaatccaaatcctggctgactacctgaccctgcaagagcactacagcagcctgaagggtctgaccctgtcatggattggcgatggaaacaatattctgcactccatcatgatgtccgccgcgaagttcggaatgcatctgcaagccgccactccaaaaggatacgaaccggatgcatccgtgaccaagttggcggaacagtacgcgaaggagaacggaaccaagctcctgctgactaacgacccgctcgaggctgcgcatgggggtaacgtgctgattacggacacctggatctccatggggcaggaggaagagaagaagaagagactgcaggcattccaggggtaccaggtcaccatgaaaaccgcaaaagtggcagcttcggactggactttcctgcattgcctgccgaggaagccggaggaagtcgacgacgaagtgttctactcgcctcggtccctggtgttccccgaggccgaaaaccggaagtggaccatcatggccgtgatggtgtccttgctgactgactatagcccgcagctgcagaagcctaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 42 - RNA, 1365 nt, synthetic construct
  misc_feature 1..1365: note = Synthetic construct
  source 1..1365: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccatgcttttcaacctgagaatcctcttgaacaatgctgcttttcggaatggccacaactttatggttcggaacttccgttgcggccagcctttacaaaacaaggtccagctgaagggccgggatttgctcacactaaagaactttactggagaagagatcaagtacatgctatggctgtcggccgacctgaagttccgtatcaagcagaagggagaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctcgataccttggcaaaggaggcttccattcccatcatcaacggcctgagcgacctgtaccacccaatccaaatcctggctgactacctgaccctgcaagagcactacagcagcctgaagggtctgaccctgtcatggattggcgatggaaacaatattctgcactccatcatgatgtccgccgcgaagttcggaatgcatctgcaagccgccactccaaaaggatacgaaccggatgcgtccgtgaccaagttggcggaacagtacgcgaaggagaacggaaccaagcttctgctgactaacgaccccctcgaggctgcgcatgggggcaacgtgctgattaccgacacctggatctccatggggcaggaggaagagaagaagaagagactgcaggcattccaggggtaccaggtcaccatgaaaaccgcaaaagtggcagcttcggactggactttcctgcattgcctgccgaggaagccggaggaagtcgacgacgaagtgttctactcgcctcggtccctggtgttccccgaggccgaaaaccggaagtggaccatcatggccgtgatggtgtccttgctgactgactatagcccgcagctgcagaagcctaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 43 - RNA, 1371 nt, synthetic construct
  misc_feature 1..1371: note = Synthetic construct
  source 1..1371: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctgttcaacctgcgaatcctgctgaacaacgccgcttttcggaacgggcacaactttatggtgaggaactttcgctgcggacagcccctccagaataaggtccagctgaagggcagggacctgctgaccctgaaaaatttcacaggggaggaaatcaagtatatgctgtggctgtcagctgatctgaagttccggatcaagcagaagggcgaatatctgcctctgctccagggcaaaagcctggggatgatcttcgaaaagcgcagtactcggaccagactgtcaaccgagactggattcgctctgctgggaggacacccttgttttctgaccactcaggacattcacctgggagtgaacgagtccctgaccgacactgctcgcgtcctgagctctatggccgacgctgtgctggctcgagtctacaaacagtccgacctggataccctggccaaggaagcttctatcccaattattaacggcctgtcagacctgtatcaccccatccagattctggccgattacctgaccctccaggagcactattctagtctgaaagggctgacactgagttggattggggacggaaacaatatcctgcactctattatgatgtcagccgccaagtttggaatgcacctccaggctgcaaccccaaaaggctacgaacccgatgcctcagtgacaaagctggctgaacagtacgccaaagagaacggcactaagctgctgctgaccaacgaccctctggaggccgctcacggaggcaacgtgctgatcaccgatacctggattagtatgggacaggaggaagagaagaagaagcggctccaggccttccagggctaccaggtgacaatgaaaaccgctaaggtcgcagccagcgattggacctttctgcactgcctgcccagaaagcccgaagaggtggacgacgaggtcttctactctcccagaagcctggtgtttcccgaagctgagaataggaagtggacaattatggcagtgatggtcagcctgctgactgattattcacctcagctccagaaaccaaagttctgataactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 44 - RNA, 1371 nt, synthetic construct
  misc_feature 1..1371: note = Synthetic construct
  source 1..1371: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcctggccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgataactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 45 - RNA, 1371 nt, synthetic construct
  misc_feature 1..1371: note = Synthetic construct
  source 1..1371: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctgttcaacctgcgaatcctgctgaacaacgccgcttttcggaacgggcacaactttatggtgaggaactttcgctgcggacagcccctccagaataaggtccagctgaagggcagggacctgctgaccctgaaaaatttcacaggggaggaaatcaagtatatgctgtggctgtcagctgatctgaagttccggatcaagcagaagggcgaatatctgcctctgctccagggcaaaagcctggggatgatcttcgaaaagcgcagtactcggaccagactgtcaaccgagactggattcgctctgctgggaggacacccttgttttctgaccactcaggacattcacctgggagtgaacgagtccctgaccgacactgctcgcgtcctgagctctatggccgacgctgtgctagctcgagtctacaaacagtccgacctggataccctggccaaggaagcttctatcccaattattaacggcctgtcagacctgtatcaccccatccagattctggccgattacctgaccctccaggagcactattctagtctgaaagggctgacactgagttggattggggacggaaacaatatcctgcactctattatgatgtcagccgccaagtttggaatgcacctccaggctgcaaccccaaaaggctacgaacccgatgcctcagtgacaaagctggctgaacagtacgccaaagagaacggcactaagctgctgctgaccaacgaccctctggaggccgctcacggaggcaacgtgctgatcaccgatacctggattagtatgggacaggaggaagagaagaagaagcggctccaggccttccagggctaccaggtgacaatgaaaaccgctaaggtcgcagccagcgattggacctttctgcactgcctgcccagaaagcccgaagaggtggacgacgaggtcttctactctcccagaagcctggtgtttcccgaagctgagaataggaagtggacaattatggcagtgatggtcagcctgctgactgattattcacctcagctccagaaaccaaagttctgataactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 46 - RNA, 1371 nt, synthetic construct
  misc_feature 1..1371: note = Synthetic construct
  source 1..1371: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctgttcaacctgcgaatcctgctgaacaatgccgcttttcggaacgggcacaatttcatggtgaggaactttcgctgcggacagcccctccagaacaaggtccagctgaagggcagggacctgctgaccctgaaaaatttcacaggggaggaaatcaagtacatgctgtggctgtcagccgatctgaagttccggatcaagcagaagggcgaatatctgcctctgctccagggcaaaagcctggggatgatcttcgaaaagcgcagtactcggaccagactgtcaacagagactggattcgcactgctgggaggacacccatgttttctgaccacacaggacattcatctgggagtgaacgagtccctgaccgacacagcacgcgtcctgagctccatggctgatgcagtgctggctcgagtctacaaacagtctgacctggataccctggccaaggaagcttctatcccaatcattaatggcctgagtgacctgtatcaccccatccagattctggccgattacctgaccctccaggagcattattctagtctgaaagggctgacactgagctggattggggacggaaacaatatcctgcactccattatgatgagcgccgccaagtttggaatgcacctccaggctgcaaccccaaaaggctacgaacccgatgcctccgtgacaaagctggcagaacagtatgccaaagagaacggcactaagctgctgctgaccaatgaccctctggaggccgctcacggaggcaacgtgctgatcactgatacctggattagtatgggacaggaggaagagaagaagaagcggctccaggccttccagggctaccaggtgacaatgaaaactgctaaggtcgcagccagcgactggacctttctgcattgcctgcccagaaagcctgaagaggtggacgatgaggtcttctactcacccagaagcctggtgtttcctgaagctgagaataggaagtggacaatcatggcagtgatggtcagcctgctgactgattattcccctcagctccagaaaccaaagttctgataactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 47 - RNA, 1368 nt, synthetic construct
  misc_feature 1..1368: note = Synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgcttttcaaccttcgcattctcctcaacaacgccgcgtttagaaacggacacaacttcatggtccgcaacttccgctgcggacagccgctgcagaacaaggtccagctcaagggtcgggatctcctgacgctgaagaactttaccggcgaagagattaagtacatgctgtggctgtccgccgaccttaagttccggatcaagcagaagggcgaataccttcccctgctgcaaggaaagtccctgggcatgatcttcgagaagcgcagtaccagaaccagactctccactgaaaccgggttcgcgctgcttggcggccacccgtgtttcctcactacgcaagacatccatcttggcgtgaacgagtcccttaccgacaccgccagggtgctgtcaagcatggccgacgccgtccttgcgcgcgtgtacaagcagtcagaccttgatactctggccaaggaagcctccatccctattatcaacggcctatccgacctttaccacccgatccagatcctcgctgactacctgaccctgcaagaacactacagcagcctcaagggactgactctgtcctggatcggcgacgggaacaacatcctgcactcaatcatgatgagcgcagccaagttcggcatgcatctccaagccgctacacccaagggttatgaaccggacgcctctgtgaccaagttggcagaacagtacgccaaggagaacggtactaagctccttttaaccaacgaccccctcgaagcagcccatggcgggaatgtgctcattaccgatacctggatttcgatgggccaggaggaggagaagaagaagcggctgcaggcgttccagggctaccaggtcaccatgaaaactgccaaagtggccgcctcggattggacctttctccactgcctgcctcggaagcctgaggaggtggacgacgaagtgttctactccccacggtccctcgtgttccccgaggccgaaaataggaagtggaccatcatggccgtgatggtgtccctcttgaccgattacagcccgcagcttcagaagcctaaattctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 48 - RNA, 1368 nt, synthetic construct
  misc_feature 1..1368: note = Synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgcttttcaatcttcgcatcctgttgaacaacgccgccttccgcaatggtcacaacttcatggtccggaacttcagatgtggacagcctctccaaaacaaggtccagctgaagggaagggacctcttaaccctcaaaaactttactggagaggagatcaagtacatgctgtggcttagcgccgaccttaagttccggatcaagcagaagggagagtacctcccgctgctgcaaggaaagagtcttggaatgatcttcgagaagcggtccaccagaactcgcctctccactgaaaccggattcgcactcctgggtggacacccgtgctttctgaccacccaagacatccacctcggagtgaacgagagcctcacggacaccgcgagagtgctgtcatccatggccgacgccgtgcttgcacgggtctacaagcagtccgatctggacactcttgccaaggaagcctccattcctatcattaacggtctgtcggatctgtaccacccgattcagatccttgcggactacctcacacttcaagaacactattcaagcctaaagggtctgaccctgtcctggatcggagatggaaacaacattctccattccatcatgatgagcgctgccaagttcggaatgcatctccaagcagcgactcctaagggttacgagccggacgcctcagtgactaagctggccgagcagtacgccaaggagaacggtaccaaactgttgcttactaacgacccgcttgaagcggcccatggaggaaacgtgctgattaccgacacctggatttcgatgggacaggaagaggagaagaagaagcggctccaggcgttccagggataccaggtcaccatgaaaacggccaaagtggccgctagcgattggacctttctgcactgcctcccgcgcaagcctgaagaagtggacgacgaagtgttctactcccctcgctctcttgtgttcccggaagccgaaaacaggaagtggaccatcatggccgtgatggtgtccctcctgaccgattacagcccgcagctgcagaagcctaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 49 - RNA, 1368 nt, synthetic construct
  misc_feature 1..1368: note = Synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgcttttcaatctccgcatcctcctcaacaacgccgcgtttagaaacggccacaacttcatggtccggaacttcagatgtggccagccgcttcagaacaaggtccagctcaagggccgggatcttctgaccctgaagaactttactggcgaagaaatcaagtacatgctctggctctccgccgacttgaagttccgcattaagcagaagggggaataccttccgctgctgcaaggaaagtcgctcggcatgatctttgagaagcgctcaacccgcaccaggctgtccactgaaaccgggttcgcgctgcttggtggccacccctgcttcctgaccacccaagacattcacctcggagtgaacgaatcgctcactgatactgcccgggtgctgtcgtcgatggccgatgcagtgctggccagggtgtacaaacagtccgatctggacactctggccaaggaggcgtccatccctattatcaacggcctttccgacctctaccacccgattcagatccttgccgattacctcaccctgcaagaacactactcgtcactgaagggtctgaccttgtcctggatcggcgacggcaacaacatcctccattccattatgatgtccgccgccaaattcggcatgcatcttcaagccgcaacccctaagggttacgagccggacgcttccgtgaccaagctcgccgagcagtacgctaaggagaacggaaccaagcttctgctgactaacgaccccctagaggcagcccacgggggcaacgtgcttattactgacacctggatctccatgggacaggaagaagagaagaagaagcggttacaggcgttccagggctatcaggtcaccatgaaaaccgccaaggtcgctgcctcggactggaccttcctgcattgcctgcctcgcaagcccgaagaagtggacgacgaggtgttctactcgccacggtcccttgtgttccctgaggccgagaatagaaagtggaccattatggccgtgatggtgtcccttctcaccgactactcgccgcaactgcagaaacccaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 50 - RNA, 1368 nt, synthetic construct
  misc_feature 1..1368: note = Synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgcttttcaatcttcgcatcctcctcaacaacgccgccttccggaacggtcacaacttcatggtccggaacttccgctgcggccagccgctccaaaacaaagtgcagcttaagggccgcgatctcctgaccctgaagaacttcaccggagaggaaatcaagtacatgctgtggctctcggcggacctgaagtttaggattaagcagaagggggagtatctgccgctgctccaagggaagtcccttggcatgatcttcgaaaagaggtccacccggactcggctcagcaccgaaacaggttttgcacttctggggggccacccgtgcttcctgacgacccaggacatccatctgggtgtcaacgagagtttgaccgacactgccagagtgctgtcatccatggcggacgcggtgctcgcgagagtgtacaagcagtccgatcttgacaccctggcaaaagaggcttcaatcccgatcattaacggactctcggatctgtaccaccctatccaaatcttggccgactacctgaccctgcaagaacactacagctccctgaagggcctgactctttcctggattggcgatggaaacaacattctccattctattatgatgtccgccgccaagttcggcatgcaccttcaagccgccaccccgaagggctacgaacctgacgcctccgtgactaagctagccgaacagtacgctaaggagaacggcactaagcttctccttaccaacgatccgctggaggcggcccatggcggaaatgtgcttatcaccgacacctggattagcatggggcaggaagaagagaagaagaaacggctccaggcattccagggctaccaggtcaccatgaaaactgccaaggtcgccgctagcgactggaccttcctccactgtctgcctcgcaagcctgaagaagtggacgacgaggtgttctactccccgcgctccctcgtgtttcctgaggccgagaacagaaagtggaccatcatggccgtgatggtgtcattacttacggactacagcccgcagctgcagaagccgaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 51 - RNA, 1368 nt, synthetic construct
  misc_feature 1..1368: note = Synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctttttaacttgagaatccttctgaacaacgccgctttccgcaacggtcataacttcatggtccggaacttcagatgtggccagcccctccaaaacaaagtgcagctgaagggccgggaccttcttacgctgaagaatttcaccggcgaagaaatcaagtacatgctctggctgtccgccgatcttaagttccgcattaagcagaagggggaatacctcccgctgctgcaagggaagtcgctgggcatgatttttgagaagcggtcaactcgcacccgcctgtccactgaaactggattcgcactgctcggtggccatccctgcttcctgaccacccaagacatccacctcggcgtgaacgagtccctgactgacaccgcccgggtcttatcctcgatggccgatgctgtgcttgcgagggtgtacaagcagtccgacctcgacacactcgcgaaggaggcctccatccccatcatcaacggcctgtccgacctttaccacccaattcagatcctcgccgattacctgaccctgcaagagcactactcgtcgctcaaggggcttaccctctcgtggattggcgacggcaacaacatccttcactccatcatgatgtcggcagcgaagttcggcatgcatctgcaagccgccacgcctaagggttatgaaccggatgcctcagtgaccaagctcgccgaacagtacgcgaaagagaatggaaccaagctacttctgaccaacgaccccctggaggccgctcacggcggcaacgtcctcattaccgatacttggatttcgatgggacaggaagaggaaaagaagaagagactgcaggcgttccagggataccaggtcaccatgaaaactgccaaagtggcagcctccgactggaccttccttcactgcctgccgaggaagcctgaagaggtggacgacgaggtgttctactccccgcgctccttggtgtttcctgaggccgaaaaccggaagtggactatcatggccgtgatggtgtccctcctcaccgactactcgccgcaactgcagaagcctaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 52 - RNA, 1368 nt, synthetic construct
  misc_feature 1..1368: note = Synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgttattcaaccttagaattctccttaacaacgccgccttccggaatgggcataactttatggtccgcaatttccgctgtggacagcctctgcaaaacaaggtccagctcaagggccgggatctgctgactctcaagaacttcactggggaagaaatcaagtacatgctctggctgagcgccgacctcaagttccgcatcaagcagaagggagagtacctcccgctgctccaagggaagtccctgggcatgatcttcgagaagagatccacccgcaccagactttccactgagactggcttcgccttgctgggaggccacccatgcttcctgacgacccaggacattcaccttggcgtgaacgagtccctgactgacaccgcaagggtgttgtcctcgatggccgacgccgtgcttgcccgggtgtacaagcagagcgatcttgacaccctggctaaggaagcttccattcccatcatcaacggtctgagcgacctgtaccacccgattcagatcctggcggactacctaaccctgcaagagcactatagctccctgaagggcctcacactttcatggatcggcgacggcaacaacatcctgcactctattatgatgagcgctgccaaattcggcatgcacctccaagccgccacgcctaaaggctacgagcccgacgcctcggtgaccaagcttgcggagcagtacgcgaaggaaaacggcaccaagctgcttctcaccaacgatcctctggaagcggcccatggtggcaacgtgctcattaccgacacttggatctccatgggacaggaggaggaaaagaagaagcggctccaggcgtttcagggttaccaggtcaccatgaaaaccgccaaggtcgcagcctccgactggaccttccttcattgccttccgcgcaagcccgaagaagtggacgatgaagtgttttactcacctcggtcactcgtgttcccggaagcagagaacaggaaatggaccattatggccgtgatggtgtccctgctcaccgattacagtccgcaactgcagaagcccaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 53 - RNA, 1368 nt, synthetic construct
  misc_feature 1..1368: note = Synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgcttttcaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcaaatcctggccgattacctcaccctgcaagaacactacagctccctgaagggtctgacattgtcctggatcggcgacggcaacaacattctccattccatcatgatgtccgccgcaaaattcggcatgcatcttcaagccgccacgcctaagggttacgaacccgacgcttccgtgactaagctcgccgagcagtacgctaaggagaacggaaccaagctgctgctgactaacgacccgctagaagcagcccacgggggcaacgtgcttattactgacacctggatctccatgggccaggaggaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcacttctcaccgactacagcccgcagcttcagaagcccaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 54 - RNA, 1368 nt, synthetic construct
  misc_feature 1..1368: note = Synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgcttttcaatctccgcatcctccttaacaacgccgcgtttagaaacggccacaacttcatggtccggaacttcagatgtggccagccgcttcaaaacaaggtccagcttaagggccgggatctcctcacccttaaaaacttcaccggcgaagagatcaagtacatgctctggctctccgcggaccttaagttccgcattaagcagaagggggaataccttccgctgcttcaaggaaagagcctcggcatgatctttgagaagcgctcaaccaggaccaggctttctactgaaactgggttcgcgcttctcggcggtcatccctgcttcctcacgacccaagacatccacctcggagtgaacgaatccctcacggatactgcccgcgtgctttcgagcatggcagacgccgtgctcgcccgggtgtacaaacagtccgatctcgacactctcgccaaggaggcgtcaattcctattatcaacggtcttagtgacctttaccacccgatccagatcctcgccgattacctcacactccaagaacactacagctcccttaagggtcttaccctctcctggatcggcgacggcaacaacattctccactccatcatgatgtccgccgcaaagttcggcatgcatcttcaagccgccaccccgaagggctacgagcctgatgcttccgtgactaagctcgccgagcagtacgctaaggagaacggaaccaagcttcttctcactaacgacccactcgaagcagcccatgggggcaacgtgcttatcactgacacctggatctccatgggccaggaagaagagaagaagaagcggctccaggcgttccagggatatcaggtcaccatgaaaaccgccaaggtcgctgcctccgactggacctttctccactgcctccctcgcaaacctgaagaagtggacgacgaggtgttctactcgccccggagcctcgtgttccccgaggccgagaatagaaagtggaccattatggccgtgatggtgtcactcctcaccgactacagcccgcagcttcagaagcccaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 55 - RNA, 1368 nt, synthetic construct
  misc_feature 1..1368: note = Synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgcttttcaatctccgcatcctccttaacaacgccgcgtttagaaacggacataacttcatggtccggaacttcagatgtggacagccgcttcaaaacaaggtccagctgaagggtcgggatcttctgaccctgaagaactttaccggagaagagatcaagtacatgctctggctctccgcggacttgaagttccgcattaagcagaagggagaatacctcccgctgcttcaaggaaagagcctcggaatgatttttgagaagcgctcaaccaggacccgcctttctactgaaactggattcgcgctgctgggtggacacccctgcttcctgacgacccaggacatccacctcggagtgaacgaatccctcactgataccgcccgggtgttatcgagcatggcagatgccgtgctggccagggtgtacaaacagtccgatctggacactctggccaaggaggcgtcaattcctatcatcaacggacttagtgacctctaccatccgattcaaatcctggccgactacctcaccctgcaagaacactacagctccctgaagggtctgacattgtcctggatcggagatggaaacaacattctccactccatcatgatgtccgccgcaaaattcggaatgcatcttcaagccgccacgcctaagggttacgaacccgacgcttccgtgactaagctcgccgagcagtacgctaaggagaacggtaccaagcttctcctgaccaacgacccactagaagcagcccacggtggaaacgtgcttattactgacacttggatctccatgggacaggaggaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccgcggagcctcgtgttccccgaggccgagaatagaaagtggaccatcatggccgtgatggtgtcactgctcaccgactacagcccgcagcttcagaagcccaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 56 - RNA, 1368 nt, synthetic construct
  misc_feature 1..1368: note = Synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgcttttcaacctccgcattctcctcaacaacgctgccttccggaatggacataacttcatggtccggaacttcagatgcggacagccgcttcagaacaaggtccagcttaaggggagagatctccttaccctcaaaaacttcactggcgaagaaatcaagtacatgctctggcttagtgcggatctcaagttccgcatcaagcagaagggagaatacctcccgctccttcaaggaaagagcctcggcatgatttttgagaagaggtccaccagaactcgcctttcaaccgagactgggttcgccctgcttggcggtcacccctgcttcctcactacccaagacatccacctcggcgtgaacgagagccttaccgacaccgcccgcgtgctctcctcaatggccgacgctgtgctcgcccgggtgtacaagcagtccgaccttgatactctcgccaaggaggcctccatcccaattatcaacgggctctctgatctctaccaccctatccaaatcctcgcggactacctcaccctccaagagcactatagctcgctcaagggcctcaccctttcctggattggcgacggcaacaacattcttcactcgatcatgatgtccgccgccaagttcggcatgcatctccaagccgcgacccccaagggctacgagcctgacgcatccgtgaccaagctcgccgagcagtacgcgaaggaaaatggcaccaagcttcttctcaccaacgacccccttgaggccgctcatggcggcaacgtgctcatcactgacacttggatcagcatgggccaggaggaggaaaagaagaagcgccttcaggcattccagggttaccaggtcaccatgaaaaccgccaaagtggccgcctccgactggacctttcttcactgtctcccgcggaagcctgaagaagtggatgacgaagtgttttactcccctcggtcactcgtgttcccggaagcagaaaacaggaagtggaccattatggcggtcatggtgtccctcctcaccgactacagcccgcagcttcagaaacccaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 57 - RNA, 1368 nt, synthetic construct
  misc_feature 1..1368: note = Synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgcttttcaatctccgcatcctccttaacaacgcagcgtttagaaacggtcacaacttcatggtccggaacttccgctgtggacagccgcttcaaaacaaggtccagctgaagggtcgggaccttctgaccctgaagaactttactggagaagagatcaagtacatgctttggctgtccgcggacttgaagttccgcattaagcagaagggagaataccttccgctgctccaaggaaagagcctgggaatgatctttgagaagcgctcaaccaggacccgcctttctactgaaactggattcgcgctgctgggtggtcacccttgcttcctgacgacccaggacattcacctcggagtgaacgagtccctcactgataccgccagagtgttatcgagcatggcagatgccgtgctggctagggtgtacaaacagtccgatctggacaccctggccaaggaggcatcaattcctattatcaacggacttagtgacctctaccatccgattcaaatcctggccgattacctcaccctgcaagaacactacagctccctgaagggtctgacattgtcctggatcggagatggaaacaacattctccattccatcatgatgtccgcggccaagttcggaatgcatctccaagccgccacgccgaaaggatacgagccggacgcttccgtgactaagctcgccgagcagtacgctaaggagaacggaaccaagcttctgctgactaacgacccgctagaagccgcccacggtggaaacgtgcttattactgacacctggatctccatgggacaggaagaagagaaaaagaagcggctgcaggcgttccagggatatcaggtcaccatgaaaaccgccaaggtcgccgcctccgactggaccttccttcactgcctgcctcggaagcctgaagaagtggacgacgaggtgttctactcgccgcggagcctcgtgttccctgaggccgagaatagaaagtggaccatcatggccgtgatggtgtcactcctcaccgactacagcccgcagcttcagaagcctaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 58 - RNA, 1368 nt, synthetic construct
  misc_feature 1..1368: note = Synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgcttttcaatctccgcattctcctcaacaacgcagcctttagaaacggccacaacttcatggtccggaacttcagatgtggccagccgcttcagaacaaggtccagctcaagggccgggacctcctcaccctcaaaaactttaccggcgaagagatcaagtacatgctctggctttcggccgaccttaagttccgcatcaagcagaagggggaataccttccgctgcttcaaggaaagtccctcggcatgatctttgaaaagcgctcgaccaggacccgcctttccactgaaaccgggttcgcgcttctcggtggccacccctgcttcctcaccacccaagacattcacctcggagtgaacgaatcccttaccgataccgcaagagtgctttcgtcgatggccgatgccgtgcttgcgcgggtgtacaagcagtcagatctcgacactctcgccaaggaggcgtccattcctattatcaacggcctttccgacctttaccacccgattcagatcctcgccgattacctcaccctgcaagagcactactcgtcactcaagggtcttaccctctcctggatcggcgacggaaacaacatcctccattcgatcatgatgtccgccgccaaattcggcatgcacctccaagccgcgaccccgaagggttacgagcccgacgcttccgtgaccaagctcgccgaacagtacgctaaggaaaacggcaccaagctcctcctcactaacgaccctctcgaagcagcccatgggggcaacgtgctcattactgacacttggatctcgatgggccaggaagaggagaaaaagaagcggcttcaggcgttccagggatatcaggtcaccatgaaaaccgccaaggtcgctgcctcggactggaccttccttcactgccttccgcgcaagcctgaagaggtggacgatgaggtgttctactccccacggtcccttgtgttccccgaggccgagaataggaagtggaccatcatggccgtgatggtgtcgctcctcactgactactccccgcaacttcagaagcctaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 59 - RNA, 1368 nt, synthetic construct
  misc_feature 1..1368: note = Synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctgtttaatctgagaatacttctaaacaacgccgccttccggaatggccataactttatggttcggaatttccgctgcggccagccgctgcagaacaaggtccagctgaagggaagagacttgctgaccctcaagaacttcaccggagaagaaatcaagtatatgctgtggctgtccgccgacctgaaattccgcatcaagcagaagggcgaatatctgccgctgttgcaagggaagtccctggggatgatcttcgagaagaggtccaccagaacacggctttcaaccgaaaccgggtttgcactgctgggtggacacccctgttttctgaccactcaagatatccacctgggcgtgaacgagtcccttaccgacactgctagggtgttgtccagcatggccgatgccgtcctggctcgcgtgtacaagcagtccgacctggataccctggcaaaggaagcgtccattcccattatcaacgggctgtccgacctgtaccatccgattcaaatcctggcggactacctgactctgcaagagcattacagcagcttgaaggggcttactctctcgtggatcggcgacgggaacaacatcctgcactccatcatgatgtccgccgccaagttcgggatgcatttgcaagctgcgaccccgaaaggttacgagcccgatgctagcgtaactaagcttgccgaacagtacgccaaagagaatggtacaaaactgcttctgactaacgacccgctggaagcagcccacggcgggaacgtgctgataaccgacacctggatttcaatggggcaggaggaagagaagaagaagcgactgcaggcgttccaaggctatcaggttaccatgaaaaccgccaaagtggcagccagcgattggactttcctgcactgtctgccgcggaagcccgaggaagttgatgacgaagtattctactcaccccggagcctcgtgttccccgaggccgaaaaccggaagtggactattatggccgtgatggtgtcgctgttgaccgactacagcccgcaactgcagaagccgaagttttagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 60 - RNA, 1368 nt, synthetic construct
  misc_feature 1..1368: note = Synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgcttttcaacctgaggatccttttgaacaacgccgcctttcgcaacggccacaactttatggtccgcaatttccgctgcgggcagccgctgcagaacaaggtccagctgaagggccgggatctgctgaccctgaagaacttcaccggggaggaaatcaagtacatgctttggctctccgccgatctgaagttcagaatcaagcagaagggagagtacctcccgttgctgcaaggaaagtcactcggaatgattttcgaaaagagaagcactaggacccgcctctcaactgaaaccgggttcgcgctgctcgggggccatccgtgtttcctgactacccaagacatccacctgggagtgaacgagtcgctgaccgacaccgcacgcgtgctgtcatccatggcggacgcagtgcttgcccgggtgtacaagcagtcggacctggacactcttgccaaggaggcatcaatccccatcattaacggactgtccgatctctaccacccgattcagatcctggctgactacctaaccctgcaagagcactactcaagcctgaaggggctgaccctgtcgtggatcggggacggcaacaacattctgcactccatcatgatgtcggcggctaagttcgggatgcatttgcaagcggcaactccgaagggttatgaacccgacgcctccgtgaccaagctggccgaacagtacgccaaggaaaacggaaccaagttgctgctgactaatgatcccctggaggcggcccacggggggaacgtgctgataaccgatacctggatctccatggggcaggaagaagagaagaaaaagcggctgcaggcattccagggataccaggtcaccatgaaaaccgcaaaagtggcagccagcgactggactttcctccattgcctgccgcgaaagccggaggaggtcgatgacgaggtgttctactccccgcggtcgctggtgttcccggaggcggaaaaccggaagtggaccattatggccgtgatggtgtcactcctgactgactacagcccgcaactgcagaagccgaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 61 - RNA, 1496 nt, synthetic construct
  misc_feature 1..1496: note = Synthetic construct
  source 1..1496: mol_type = other RNA
cttaagggggcgctgcctacggaggtggcagccatctccttctcggcatcaagcttaccatggtgccccaggccctgctcttggtcccgctgctggtgttccccctctgcttcggcaagttccccatctacaccatccccgacaagctggggccgtggagccccatcgacatccaccacctgtcctgccccaacaacctcgtggtcgaggacgagggctgcaccaacctgagcgggttctcctacatgcttttcaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagataagtgaatgcaaggctggccggaagcccttgcctgaaagcaagatttcagcctggaagagggcaaagtggacgggagtggacaggagtggatgcgataagatgtggtttgaagctgatgggtgccagccctgcattgctgagtcaatcaataaagagctttcttttgacccattctagatctag

SEQ ID NO: 62 - RNA, 1368 nt, synthetic construct
  misc_feature 1..1368: note = Synthetic construct
  source 1..1368: mol_type = other RNA
aggaaacttaagattattacatcaaaacaaaaagccgccaatgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcctggccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 63 - RNA, 1495 nt, synthetic construct
  misc_feature 1..1495: note = Synthetic construct
  source 1..1495: mol_type = other RNA
cttaagggggcgctgcctacggaggtggcagccatctccttctcggcatcaagcttaccatggtgccccaggccctgctcttggtcccgctgctggtgttccccctctgcttcggcaagttccccatctacaccatccccgacaagctggggccgtggagccccatcgacatccaccacctgtcctgccccaacaacctcgtggtcgaggacgagggctgcaccaacctgagcgggttctcctacatgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcctggccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaataagtgatgcaaggctggccggaagcccttgcctgaaagcaagatttcagcctggaagagggcaaagtggacgggagtggacaggagtggatgcgataagatgtggtttgaagctgatgggtgccagccctgcattgctgagtcaatcaataaagagctttcttttgacccattctagatctag

SEQ ID NO: 64 - RNA, 1368 nt, synthetic construct
  misc_feature 1..1368: note = Synthetic construct
  source 1..1368: mol_type = other RNA
aggaaacttaagattattacatcaaaacaaaaagccgccaatgcttttcaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 65 - RNA, 1495 nt, synthetic construct
  misc_feature 1..1495: note = Synthetic construct
  source 1..1495: mol_type = other RNA
cttaagggggcgctgcctacggaggtggcagccatctccttctcggcatcaagcttaccatggtgccccaggccctgctcttggtcccgctgctggtgttccccctctgcttcggcaagttccccatctacaccatccccgacaagctggggccgtggagccccatcgacatccaccacctgtcctgccccaacaacctcgtggtcgaggacgagggctgcaccaacctgagcgggttctcctacatgcttttcaacctgagaatcctcttgaacaatgctgcttttcggaatggccacaactttatggttcggaacttccgttgcggccagcctttacaaaacaaggtccagctgaagggccgggatttgctcacactaaagaactttactggagaagagatcaagtacatgctatggctgtcggccgacctgaagttccgtatcaagcagaagggagaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctcgataccttggcaaaggaggcttccattcccatcatcaacggcctgagcgacctgtaccacccaatccaaatcctggctgactacctgaccctgcaagagcactacagcagcctgaagggtctgaccctgtcatggattggcgatggaaacaatattctgcactccatcatgatgtccgccgcgaagttcggaatgcatctgcaagccgccactccaaaaggatacgaaccggatgcatccgtgaccaagttggcggaacagtacgcgaaggagaacggaaccaagctcctgctgactaacgacccgctcgaggctgcgcatgggggtaacgtgctgattacggacacctggatctccatggggcaggaggaagagaagaagaagagactgcaggcattccaggggtaccaggtcaccatgaaaaccgcaaaagtggcagcttcggactggactttcctgcattgcctgccgaggaagccggaggaagtcgacgacgaagtgttctactcgcctcggtccctggtgttccccgaggccgaaaaccggaagtggaccatcatggccgtgatggtgtccttgctgactgactatagcccgcagctgcagaagcctaagttctagataagtgatgcaaggctggccggaagcccttgcctgaaagcaagatttcagcctggaagagggcaaagtggacgggagtggacaggagtggatgcgataagatgtggtttgaagctgatgggtgccagccctgcattgctgagtcaatcaataaagagctttcttttgacccattctagatctag

SEQ ID NO: 66 - RNA, 1475 nt, synthetic construct
  misc_feature 1..1475: note = Synthetic construct
  source 1..1475: mol_type = other RNA
tgagtgtcgtacagcctccaggcccccccctcccgggagagccatagtggtctgcggaaccggtgagtacaccggaattgccgggaagactgggtcctttcttggataaacccactctatgcccggccatttgggcgtgcccccgcaagactgctagccgagtagtgttgggttgcgatgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcctggccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaataagtgatagagcggcaaaccctagctacactccatagctagtttcttttttttttgtttttttttttttttttttttttttttttttttttttttcctttcttttccttctttttttcctcttttcttggtggctccatcttagccctagtcacggctagctgtgaaaggtccgtgagccgcatgactgcagagagtgccgtaactggcctctctgcagatcatgttctag

SEQ ID NO: 67 - RNA, 1335 nt, synthetic construct
  misc_feature 1..1335: note = Synthetic construct
  source 1..1335: mol_type = other RNA
aggaaacttaagattattacatcaaaacaaaaagccgccaatgcttttcaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctaggctggagcctcggtagccgttcctcctgcccgctgggcctcccaacgggccctcctcccctccttgcaccggcccttcctggtctttgaataaagtctgagtgggcagcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 68 - RNA, 1335 nt, synthetic construct
  misc_feature 1..1335: note = Synthetic construct
  source 1..1335: mol_type = other RNA
aggaaacttaagattattacatcaaaacaaaaagccgccaatgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcctggccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgagctggagcctcggtagccgttcctcctgcccgctgggcctcccaacgggccctcctcccctccttgcaccggcccttcctggtctttgaataaagtctgagtgggcagcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 69 - RNA, 1335 nt, synthetic construct
  misc_feature 1..1335: note = Synthetic construct
  source 1..1335: mol_type = other RNA
aggaaacttaagattattacatcaaaacaaaaagccgccaatgcttttcaacctgagaatcctcttgaacaatgctgcttttcggaatggccacaactttatggttcggaacttccgttgcggccagcctttacaaaacaaggtccagctgaagggccgggatttgctcacactaaagaactttactggagaagagatcaagtacatgctatggctgtcggccgacctgaagttccgtatcaagcagaagggagaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctcgataccttggcaaaggaggcttccattcccatcatcaacggcctgagcgacctgtaccacccaatccaaatcctggctgactacctgaccctgcaagagcactacagcagcctgaagggtctgaccctgtcatggattggcgatggaaacaatattctgcactccatcatgatgtccgccgcgaagttcggaatgcatctgcaagccgccactccaaaaggatacgaaccggatgcatccgtgaccaagttggcggaacagtacgcgaaggagaacggaaccaagctcctgctgactaacgacccgctcgaggctgcgcatgggggtaacgtgctgattacggacacctggatctccatggggcaggaggaagagaagaagaagagactgcaggcattccaggggtaccaggtcaccatgaaaaccgcaaaagtggcagcttcggactggactttcctgcattgcctgccgaggaagccggaggaagtcgacgacgaagtgttctactcgcctcggtccctggtgttccccgaggccgaaaaccggaagtggaccatcatggccgtgatggtgtccttgctgactgactatagcccgcagctgcagaagcctaagttctaggctggagcctcggtagccgttcctcctgcccgctgggcctcccaacgggccctcctcccctccttgcaccggcccttcctggtctttgaataaagtctgagtgggcagcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 70 - RNA, 1346 nt, synthetic construct
  misc_feature 1..1346: note = Synthetic construct
  source 1..1346: mol_type = other RNA
aggaaacttaagaattattggttaaagaagtatattagtgctaatttccctccgtttgtcctagcttttctcttctgtcaaccccacacgcctttggcacaatgcttttcaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagctcgagacacatcacaaccacaaccttctcaggctaccctgagaaaaaaagacatgaagactcaggactcatcttttctgttggtgtaaaatcaacaccctaaggaacacaaatttctttaaacatttgacttcttgtctctgtgctgcaattaataaaaaatggaaagaatctatctag

SEQ ID NO: 71 - RNA, 1346 nt, synthetic construct
  misc_feature 1..1346: note = Synthetic construct
  source 1..1346: mol_type = other RNA
aggaaacttaagaattattggttaaagaagtatattagtgctaatttccctccgtttgtcctagcttttctcttctgtcaaccccacacgcctttggcacaatgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcctggccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgactcgagacacatcacaaccacaaccttctcaggctaccctgagaaaaaaagacatgaagactcaggactcatcttttctgttggtgtaaaatcaacaccctaaggaacacaaatttctttaaacatttgacttcttgtctctgtgctgcaattaataaaaaatggaaagaatctatctag

SEQ ID NO: 72 - RNA, 1346 nt, synthetic construct
  misc_feature 1..1346: note = Synthetic construct
  source 1..1346: mol_type = other RNA
aggaaacttaagaattattggttaaagaagtatattagtgctaatttccctccgtttgtcctagcttttctcttctgtcaaccccacacgcctttggcacaatgcttttcaacctgagaatcctcttgaacaatgctgcttttcggaatggccacaactttatggttcggaacttccgttgcggccagcctttacaaaacaaggtccagctgaagggccgggatttgctcacactaaagaactttactggagaagagatcaagtacatgctatggctgtcggccgacctgaagttccgtatcaagcagaagggagaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctcgataccttggcaaaggaggcttccattcccatcatcaacggcctgagcgacctgtaccacccaatccaaatcctggctgactacctgaccctgcaagagcactacagcagcctgaagggtctgaccctgtcatggattggcgatggaaacaatattctgcactccatcatgatgtccgccgcgaagttcggaatgcatctgcaagccgccactccaaaaggatacgaaccggatgcatccgtgaccaagttggcggaacagtacgcgaaggagaacggaaccaagctcctgctgactaacgacccgctcgaggctgcgcatgggggtaacgtgctgattacggacacctggatctccatggggcaggaggaagagaagaagaagagactgcaggcattccaggggtaccaggtcaccatgaaaaccgcaaaagtggcagcttcggactggactttcctgcattgcctgccgaggaagccggaggaagtcgacgacgaagtgttctactcgcctcggtccctggtgttccccgaggccgaaaaccggaagtggaccatcatggccgtgatggtgtccttgctgactgactatagcccgcagctgcagaagcctaagttctagctcgagacacatcacaaccacaaccttctcaggctaccctgagaaaaaaagacatgaagactcaggactcatcttttctgttggtgtaaaatcaacaccctaaggaacacaaatttctttaaacatttgacttcttgtctctgtgctgcaattaataaaaaatggaaagaatctatctag

SEQ ID NO: 73 - RNA, 1222 nt, synthetic construct
  misc_feature 1..1222: note = Synthetic construct
  source 1..1222: mol_type = other RNA
attattacatcaaaacaaaaagccgccaccatgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaggtctctagtaatgagctggagcctcggtagccgttcctcctgcccgctgggcctcccaacgggccctcctcccctccttgcaccggcccttcctggtctttgaataaagtctgagtgggcatctag

SEQ ID NO: 74 - RNA, 1346 nt, synthetic construct
  misc_feature 1..1346: note = Synthetic construct
  source 1..1346: mol_type = other RNA
aggaaacttaagaattattggttaaagaagtatattagtgctaatttccctccgtttgtcctagcttttctcttctgtcaaccccacacgcctttggcacaatgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgactcgagacacatcacaaccacaaccttctcaggctaccctgagaaaaaaagacatgaagactcaggactcatcttttctgttggtgtaaaatcaacaccctaaggaacacaaatttctttaaacatttgacttcttgtctctgtgctgcaattaataaaaaatggaaagaatctatctag

SEQ ID NO: 75 - RNA, 1368 nt, synthetic construct
  misc_feature 1..1368: note = Synthetic construct
  source 1..1368: mol_type = other RNA
aggaaacttaagattattacatcaaaacaaaaagccgccaatgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 76 - RNA, 1496 nt, synthetic construct
  misc_feature 1..1496: note = Synthetic construct
  source 1..1496: mol_type = other RNA
cttaagggggcgctgcctacggaggtggcagccatctccttctcggcatcaagcttaccatggtgccccaggccctgctcttggtcccgctgctggtgttccccctctgcttcggcaagttccccatctacaccatccccgacaagctggggccgtggagccccatcgacatccaccacctgtcctgccccaacaacctcgtggtcgaggacgagggctgcaccaacctgagcgggttctcctacatgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaataagtgaatgcaaggctggccggaagcccttgcctgaaagcaagatttcagcctggaagagggcaaagtggacgggagtggacaggagtggatgcgataagatgtggtttgaagctgatgggtgccagccctgcattgctgagtcaatcaataaagagctttcttttgacccattctagatctag

SEQ ID NO: 77 - RNA, 1380 nt, synthetic construct
  misc_feature 1..1380: note = Synthetic construct
  source 1..1380: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgggcgtcttcaacctgcggatcctgctgaacaacgccgccttccggaacggccacaacttcatggtccgcaacttcagatgcggccagcccctgcagaacaaggtgcagctgaagggccgggacctgctgaccctgaagaacttcaccggcgaagagatcaagtacatgctgtggctgagcgccgacctgaagttccggatcaagcagaagggcgagtacctgcccctgctgcaaggcaagagcctgggcatgatcttcgagaagcggagcacccggacccggctgagcaccgagacaggctttgccctgctgggaggccacccctgctttctgaccacccaggacatccacctgggcgtgaacgagagcctgaccgacaccgccagagtgctgagcagcatggccgacgccgtgctggcccgggtgtacaagcagagcgacctggacaccctggccaaagaggccagcatccccatcatcaacggcctgagcgacctgtaccaccccatccagatcctggccgactacctgaccctgcaggaacactacagctccctgaagggcctgaccctgagctggatcggcgacggcaacaacatcctgcacagcatcatgatgagcgccgccaagttcggcatgcatctgcaggccgccacccccaagggctacgagcctgatgccagcgtgaccaagctggccgagcagtacgccaaagagaacggcaccaagctgctgctgaccaacgaccccctggaagccgcccacggcggcaacgtgctgatcaccgacacctggatcagcatgggccaggaagaggaaaagaagaagcggctgcaggccttccagggctaccaggtcacaatgaagaccgccaaggtggccgccagcgactggaccttcctgcactgcctgccccggaagcccgaagaggtggacgacgaggtgttctacagcccccggtccctggtgttccccgaggccgagaaccggaagtggaccattatggccgtgatggtgtccctgctgaccgactactccccccagctgcagaagcccaagttctagataagtgaactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 78 - RNA, 1380 nt, synthetic construct
  misc_feature 1..1380: note = Synthetic construct
  source 1..1380: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgggcgtcttcaacctgcggatcctgctgaacaacgccgccttccggaacggccacaacttcatggtccgcaacttcagatgcggccagcccctgcagaacagggtgcagctgaagggccgggacctgctgaccctgaagaacttcaccggcgaagagatcaggtacatgctgtggctgagcgccgacctgaagttccggatcaagcagaagggcgagtacctgcccctgctgcaaggcaagagcctgggcatgatcttcgagaagcggagcacccggacccggctgagcaccgagacaggctttgccctgctgggaggccacccctgctttctgaccacccaggacatccacctgggcgtgaacgagagcctgaccgacaccgccagagtgctgagcagcatggccgacgccgtgctggcccgggtgtacaagcagagcgacctggacaccctggccaaagaggccagcatccccatcatcaacggcctgagcgacctgtaccaccccatccagatcctggccgactacctgaccctgcaggaacactacagctccctgaagggcctgaccctgagctggatcggcgacggcaacaacatcctgcacagcatcatgatgagcgccgccaagttcggcatgcatctgcaggccgccacccccaagggctacgagcctgatgccagcgtgaccaagctggccgagcagtacgccaaagagaacggcaccaagctgctgctgaccaacgaccccctggaagccgcccacggcggcaacgtgctgatcaccgacacctggatcagcatgggccaggaagaggaaaagaagaagcggctgcaggccttccagggctaccaggtcacaatgaagaccgccaaggtggccgccagcgactggaccttcctgcactgcctgccccggaagcccgaagaggtggacgacgaggtgttctacagcccccggtccctggtgttccccgaggccgagaaccggaagtggaccattatggccgtgatggtgtccctgctgaccgactactccccccagctgcagaagcccaagttctagataagtgaactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 79 - RNA, 1380 nt, synthetic construct
  misc_feature 1..1380: note = Synthetic construct
  source 1..1380: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctggtcttcaacctgcggatcctgctgaacaacgccgccttccggaacggccacaacttcatggtccgcaacttcagatgcggccagcccctgcagaacagggtgcagctgaagggccgggacctgctgaccctgaagaacttcaccggcgaagagatcaggtacatgctgtggctgagcgccgacctgaagttccggatcaagcagaagggcgagtacctgcccctgctgcaaggcaagagcctgggcatgatcttcgagaagcggagcacccggacccggctgagcaccgagacaggctttgccctgctgggaggccacccctgctttctgaccacccaggacatccacctgggcgtgaacgagagcctgaccgacaccgccagagtgctgagcagcatggccgacgccgtgctggcccgggtgtacaagcagagcgacctggacaccctggccaaagaggccagcatccccatcatcaacggcctgagcgacctgtaccaccccatccagatcctggccgactacctgaccctgcaggaacactacagctccctgaagggcctgaccctgagctggatcggcgacggcaacaacatcctgcacagcatcatgatgagcgccgccaagttcggcatgcatctgcaggccgccacccccaagggctacgagcctgatgccagcgtgaccaagctggccgagcagtacgccaaagagaacggcaccaagctgctgctgaccaacgaccccctggaagccgcccacggcggcaacgtgctgatcaccgacacctggatcagcatgggccaggaagaggaaaagaagaagcggctgcaggccttccagggctaccaggtcacaatgaagaccgccaaggtggccgccagcgactggaccttcctgcactgcctgccccggaagcccgaagaggtggacgacgaggtgttctacagcccccggtccctggtgttccccgaggccgagaaccggaagtggaccattatggccgtgatggtgtccctgctgaccgactactccccccagctgcagaagcccaagttctagataagtgaactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 80 - RNA, 1377 nt, synthetic construct
  misc_feature 1..1377: note = Synthetic construct
  source 1..1377: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctgttcaacctgaggatcctgctgaacaacgcagctttcaggaacggccacaacttcatggtgaggaacttccggtgcggccagcccctgcagaacaaggtgcagctgaagggcagggacctgctgaccctgaagaacttcaccggagaggagatcaagtacatgctgtggctgagcgcagacctgaagttcaggatcaagcagaagggagagtacctgcccctgctgcaggggaagtccctgggcatgatcttcgagaagaggagtaccaggaccaggctgagcaccgaaaccggcttcgccctgctgggaggacacccctgcttcctgaccacccaggacatccacctgggcgtgaacgagagtctgaccgacaccgccagggtgctgtctagcatggccgacgccgtgctggccagggtgtacaagcagtcagacctggacaccctggctaaggaggccagcatccccatcatcaacggcctgagcgacctgtaccaccccatccagatcctggctgactacctgaccctgcaggagcactacagctctctgaagggcctgaccctgagctggatcggcgacgggaacaacatcctgcacagcatcatgatgagcgccgccaagttcggcatgcacctgcaggccgctacccccaagggttacgagcccgacgccagcgtgaccaagctggcagagcagtacgccaaggagaacggcaccaagctgctgctgaccaacgaccccctggaggccgcccacggaggcaacgtgctgatcaccgacacctggatcagcatgggacaggaggaggagaagaagaagcggctgcaggctttccagggttaccaggtgaccatgaagaccgccaaggtggctgccagcgactggaccttcctgcactgcctgcccaggaagcccgaggaggtggacgacgaggtgttctactctcccaggagcctggtgttccccgaggccgagaacaggaagtggaccatcatggctgtgatggtgtccctgctgaccgactacagcccccagctgcagaagcccaagttctgaataagtgaactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 81 - RNA, 1377 nt, synthetic construct
  misc_feature 1..1377: note = Synthetic construct
  source 1..1377: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctgttcaacctgaggatcctgctgaacaacgcagctttcaggaacggccacaacttcatggtgaggaacttccggtgcggccagcccctgcagaacaaggtgcagctgaagggcagggacctgctgaccctgaagaacttcaccggagaggagatcaagtacatgctgtggctgagcgcagacctgaagttcaggatcaagcagaagggagagtacctgcccctgctgcaggggaagtccctgggcatgatcttcgagaagaggagtaccaggaccaggctgagcaccgaaaccggcttcgccctgctgggaggacacccctgcttcctgacgacccaggacatccacctcggagtgaacgaatccctcaccgataccgcccgggtgttatcaagcatggcagatgccgtgctggccagggtgtacaaacagtccgatctggacactctggctaaggaggccagcatccccatcatcaacggcctgagcgacctgtaccaccccatccagatcctggctgactacctgaccctgcaggagcactacagctctctgaagggcctgaccctgagctggatcggcgacgggaacaacatcctgcactccatcatgatgtccgccgcgaagttcggaatgcatctgcaagccgccacgccaaaaggatacgaaccggatgcgcccgtgacaaagttggcggaacagtacgctaaggagaacggaaccaagctgctgctgaccaacgaccccctggaggccgcccacggaggcaacgtgctgatcaccgacacctggatcagcatgggacaggaggaggagaagaagaagcggctgcaggctttccagggttaccaggtgaccatgaagaccgccaaggtggctgccagcgactggaccttcctgcactgcctgcccaggaagcccgaggaggtggacgacgaggtgttctactctcccaggagcctggtgttccccgaggccgagaacaggaagtggaccatcatggctgtgatggtgtccctgctgaccgactacagcccccagctgcagaagcccaagttctgaataagtgaactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 82 - RNA, 1477 nt, synthetic construct
  misc_feature 1..1477: note = Synthetic construct
  source 1..1477: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaataagtgaactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 83 - RNA, 1371 nt, synthetic construct
  misc_feature 1..1371: note = Synthetic construct
  source 1..1371: mol_type = other RNA
aggaaacttaagtcaacacaacatatacaaaacaaacgaatctcaagcaatcaagcattctacttctattgcagcaatttaaatcatttcttttaaagcaaaagcaattttctgaaaattttcaccatttacgaacgatagccatgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgactagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 84 - RNA, 1371 nt, synthetic construct
  misc_feature 1..1371: note = Synthetic construct
  source 1..1371: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgcttttcaacttgagaatcctgctgaacaacgccgcctttcgcaacggtcacaattttatggtcagaaacttcagatgcggacagcccctccaaaacaaggtccagctgaagggccgcgatctcctcaccctgaagaacttcacgggggaggagatcaagtacatgctgtggctctccgctgacctgaagttcaggatcaagcagaagggagaatatctgccgctgctgcaagggaagtccctggggatgattttcgagaagcggagcacccggactcggctctccactgaaactggtttcgcccttctgggcggtcacccctgcttcctgaccactcaagacattcacctcggagtgaacgagtccttgactgacaccgcccgggtgctgtcgagcatggcagacgccgtgctagcccgcgtgtacaagcagtcagacctcgataccctggccaaggaggcttcgatcccgatcatcaacgggttgtccgacctgtaccacccgattcagattctcgccgactacctcaccctgcaagagcattacagctccctgaaggggcttaccctgtcctggattggcgacggaaacaacatcctgcactccattatgatgtcggcggccaagttcggcatgcacctccaagccgcgacccctaagggttacgaaccagacgcgtcagtgactaagctggccgaacagtacgcaaaggaaaatggcacgaagctgctcctgaccaacgatccgttggaagccgcccatggcggaaatgtgctcatcaccgacacctggatctcgatgggacaggaggaagagaagaagaagcggctgcaggcgttccagggctaccaggtcaccatgaaaactgccaaggtggccgccagcgactggaccttcctgcactgccttccgcgcaagcctgaggaggtggacgatgaagtgttctactctccacggtccctggtgttccccgaggcggagaaccgcaaatggaccatcatggctgtgatggtcagcctgctgaccgattacagccctcagttgcaaaagccgaagttttgataactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 85 - RNA, 1471 nt, synthetic construct
  misc_feature 1..1471: note = Synthetic construct
  source 1..1471: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctgttcaacctccgcatcctcctcaacaacgccgcattcagaaacgggcacaacttcatggtcagaaacttccgctgcgggcaacccctacaaaacaaggtccagctcaaggggcgggacctcctgaccctgaagaacttcaccggcgaagagatcaagtacatgctgtggctctccgccgacctgaagttccgcatcaagcagaagggagagtacctcccgctgctgcaagggaagtcgctggggatgatcttcgagaagcggtcaaccagaacccggctgtcaaccgaaaccgggttcgcactgctggggggacacccgtgcttcctgaccacccaagacatccacctgggagtgaacgaatcgctgaccgacaccgcccgcgtgctgagctcaatggcggacgccgtgctggcccgcgtgtacaagcagtccgacctggacaccctggccaaggaagcgtccatcccgatcatcaacggactgtccgacctgtaccacccgatccagatcctggcagactacctgaccctgcaagaacactacagctccctgaagggcctgaccctgtcatggatcggggacgggaacaacatcctgcactccataatgatgtcagccgccaagttcggaatgcacctccaagccgcaaccccgaagggctacgaaccggacgcatcagtgaccaaactggccgagcagtacgccaaggaaaacggcaccaagctcctgctgaccaacgacccgctggaggccgcacacggggggaacgtgctgatcaccgacacctggatctccatgggacaggaggaggaaaagaagaagcggctgcaggcgttccaggggtaccaggtcaccatgaaaaccgcgaaggtcgcggcatcagactggaccttcctgcactgcctgccccggaagccggaagaggtggacgacgaggtgttctactcgccgcgctcgctggtgttccccgaggcggagaacaggaagtggaccatcatggcggtgatggtcagcctcctgaccgactactcgccgcagctgcagaagccgaagttctgataactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 86 - RNA, 1771 nt, synthetic construct
  misc_feature 1..1771: note = Synthetic construct
  source 1..1771: mol_type = other RNA
ctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacgtgtgtttagtcgaggttaaaaaacgtctaggccccccgaaccacggggacgtggttttcctttgaaaaacacgatgataatatgcttttcaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctgaataagtagatagtgcagtcactggcacaacgcgttgcccggtaagccaatcgggtatacacggtcgtcatactgcagacagggttcttctactttgcaagatagtctagagtagtaaaataaatagtataagtctag

SEQ ID NO: 87 - RNA, 1771 nt, synthetic construct
  misc_feature 1..1771: note = Synthetic construct
  source 1..1771: mol_type = other RNA
ctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacgtgtgtttagtcgaggttaaaaaacgtctaggccccccgaaccacggggacgtggttttcctttgaaaaacacgatgataatatgcttttcaacctgagaatcctcttgaacaatgctgcttttcggaatggccacaactttatggttcggaacttccgttgcggccagcctttacaaaacaaggtccagctgaagggccgggatttgctcacactaaagaactttactggagaagagatcaagtacatgctatggctgtcggccgacctgaagttccgtatcaagcagaagggagaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctcgataccttggcaaaggaggcttccattcccatcatcaacggcctgagcgacctgtaccacccaatccaaatcctggctgactacctgaccctgcaagagcactacagcagcctgaagggtctgaccctgtcatggattggcgatggaaacaatattctgcactccatcatgatgtccgccgcgaagttcggaatgcatctgcaagccgccactccaaaaggatacgaaccggatgcatccgtgaccaagttggcggaacagtacgcgaaggagaacggaaccaagctcctgctgactaacgacccgctcgaggctgcgcatgggggtaacgtgctgattacggacacctggatctccatggggcaggaggaagagaagaagaagagactgcaggcattccaggggtaccaggtcaccatgaaaaccgcaaaagtggcagcttcggactggactttcctgcattgcctgccgaggaagccggaggaagtcgacgacgaagtgttctactcgcctcggtccctggtgttccccgaggccgaaaaccggaagtggaccatcatggccgtgatggtgtccttgctgactgactatagcccgcagctgcagaagcctaagttctgaataagtagatagtgcagtcactggcacaacgcgttgcccggtaagccaatcgggtatacacggtcgtcatactgcagacagggttcttctactttgcaagatagtctagagtagtaaaataaatagtataagtctag

SEQ ID NO: 88 - RNA, 1771 nt, synthetic construct
  misc_feature 1..1771: note = Synthetic construct
  source 1..1771: mol_type = other RNA
ctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacgtgtgtttagtcgaggttaaaaaacgtctaggccccccgaaccacggggacgtggttttcctttgaaaaacacgatgataatatgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcctggccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaataagtagatagtgcagtcactggcacaacgcgttgcccggtaagccaatcgggtatacacggtcgtcatactgcagacagggttcttctactttgcaagatagtctagagtagtaaaataaatagtataagtctag

SEQ ID NO: 89 - RNA, 1368 nt, synthetic construct; identical to SEQ ID NO: 34
  misc_feature 1..1368: note = Synthetic construct
  source 1..1368: mol_type = other RNA

SEQ ID NO: 90 - RNA, 1368 nt, synthetic construct; identical to SEQ ID NO: 34
  misc_feature 1..1368: note = Synthetic construct
  source 1..1368: mol_type = other RNA

SEQ ID NO: 91 - RNA, 1368 nt, synthetic construct; identical to SEQ ID NO: 34
  misc_feature 1..1368: note = Synthetic construct
  source 1..1368: mol_type = other RNA

SEQ ID NO: 92 - RNA, 1368 nt, synthetic construct; identical to SEQ ID NO: 34
  misc_feature 1..1368: note = Synthetic construct
  source 1..1368: mol_type = other RNA

SEQ ID NO: 93 - RNA, 1368 nt, synthetic construct; identical to SEQ ID NO: 34
  misc_feature 1..1368: note = Synthetic construct
  source 1..1368: mol_type = other RNA

SEQ ID NO: 94 - RNA, 1371 nt, synthetic construct
  misc_feature 1..1371: note = Synthetic construct
  source 1..1371: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatggtgttcaacctccgcatcctcctcaacaacgccgcattcagaaacgggcacaacttcatggtcagaaacttccgctgcgggcaacccctacaaaacaaggtccagctcaaggggcgggacctcctgaccctgaagaacttcaccggcgaagagatcaagtacatgctgtggctctccgccgacctgaagttccgcatcaagcagaagggagagtacctcccgctgctgcaagggaagtcgctggggatgatcttcgagaagcggtcaaccagaacccggctgtcaaccgaaaccgggttcgcactgctggggggacacccgtgcttcctgaccacccaagacatccacctgggagtgaacgaatcgctgaccgacaccgcccgcgtgctgagctcaatggcggacgccgtgctggcccgcgtgtacaagcagtccgacctggacaccctggccaaggaagcgtccatcccgatcatcaacggactgtccgacctgtaccacccgatccagatcctggcagactacctgaccctgcaagaacactacagctccctgaagggcctgaccctgtcatggatcggggacgggaacaacatcctgcactccataatgatgtcagccgccaagttcggaatgcacctccaagccgcaaccccgaagggctacgaaccggacgcatcagtgaccaaactggccgagcagtacgccaaggaaaacggcaccaagctcctgctgaccaacgacccgctggaggccgcacacggggggaacgtgctgatcaccgacacctggatctccatgggacaggaggaggaaaagaagaagcggctgcaggcgttccaggggtaccaggtcaccatgaaaaccgcgaaggtcgcggcatcagactggaccttcctgcactgcctgccccggaagccggaagaggtggacgacgaggtgttctactcgccgcgctcgctggtgttccccgaggcggagaacaggaagtggaccatcatggcggtgatggtcagcctcctgaccgactactcgccgcagctgcagaagccgaagttctgataactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 95 - RNA, 1371 nt, synthetic construct
  misc_feature 1..1371: note = Synthetic construct
  source 1..1371: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatggtgttcaacctccgcatcctcctcaacaacgccgcattcagaaacgggcacaacttcatggtcagaaacttccgctgcgggcaacccctacaaaaccgggtccagctcaaggggcgggacctcctgaccctgaagaacttcaccggcgaagagatcaagtacatgctgtggctctccgccgacctgaagttccgcatcaagcagaagggagagtacctcccgctgctgcaagggaagtcgctggggatgatcttcgagaagcggtcaaccagaacccggctgtcaaccgaaaccgggttcgcactgctggggggacacccgtgcttcctgaccacccaagacatccacctgggagtgaacgaatcgctgaccgacaccgcccgcgtgctgagctcaatggcggacgccgtgctggcccgcgtgtacaagcagtccgacctggacaccctggccaaggaagcgtccatcccgatcatcaacggactgtccgacctgtaccacccgatccagatcctggcagactacctgaccctgcaagaacactacagctccctgaagggcctgaccctgtcatggatcggggacgggaacaacatcctgcactccataatgatgtcagccgccaagttcggaatgcacctccaagccgcaaccccgaagggctacgaaccggacgcatcagtgaccaaactggccgagcagtacgccaaggaaaacggcaccaagctcctgctgaccaacgacccgctggaggccgcacacggggggaacgtgctgatcaccgacacctggatctccatgggacaggaggaggaaaagaagaagcggctgcaggcgttccaggggtaccaggtcaccatgaaaaccgcgaaggtcgcggcatcagactggaccttcctgcactgcctgccccggaagccggaagaggtggacgacgaggtgttctactcgccgcgctcgctggtgttccccgaggcggagaacaggaagtggaccatcatggcggtgatggtcagcctcctgaccgactactcgccgcagctgcagaagccgaagttctgataactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 96 - RNA, 1371 nt, synthetic construct
  misc_feature 1..1371: note = Synthetic construct
  source 1..1371: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatggtgttcaacctccgcatcctcctcaacaacgccgcattcagaaacgggcacaacttcatggtcagaaacttccgctgcgggcaacccctacaaaaccgggtccagctcaaggggcgggacctcctgaccctgaagaacttcaccggcgaagagatccggtacatgctgtggctctccgccgacctgaagttccgcatcaagcagaagggagagtacctcccgctgctgcaagggaagtcgctggggatgatcttcgagaagcggtcaaccagaacccggctgtcaaccgaaaccgggttcgcactgctggggggacacccgtgcttcctgaccacccaagacatccacctgggagtgaacgaatcgctgaccgacaccgcccgcgtgctgagctcaatggcggacgccgtgctggcccgcgtgtacaagcagtccgacctggacaccctggccaaggaagcgtccatcccgatcatcaacggactgtccgacctgtaccacccgatccagatcctggcagactacctgaccctgcaagaacactacagctccctgaagggcctgaccctgtcatggatcggggacgggaacaacatcctgcactccataatgatgtcagccgccaagttcggaatgcacctccaagccgcaaccccgaagggctacgaaccggacgcatcagtgaccaaactggccgagcagtacgccaaggaaaacggcaccaagctcctgctgaccaacgacccgctggaggccgcacacggggggaacgtgctgatcaccgacacctggatctccatgggacaggaggaggaaaagaagaagcggctgcaggcgttccaggggtaccaggtcaccatgaaaaccgcgaaggtcgcggcatcagactggaccttcctgcactgcctgccccggaagccggaagaggtggacgacgaggtgttctactcgccgcgctcgctggtgttccccgaggcggagaacaggaagtggaccatcatggcggtgatggtcagcctcctgaccgactactcgccgcagctgcagaagccgaagttctgataactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 97 - RNA, 1371 nt, synthetic construct
  misc_feature 1..1371: note = Synthetic construct
  source 1..1371: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctggtcaacctccgcatcctcctcaacaacgccgcattcagaaacgggcacaacttcatggtcagaaacttccgctgcgggcaacccctacaaaacaaggtccagctcaaggggcgggacctcctgaccctgaagaacttcaccggcgaagagatcaagtacatgctgtggctctccgccgacctgaagttccgcatcaagcagaagggagagtacctcccgctgctgcaagggaagtcgctggggatgatcttcgagaagcggtcaaccagaacccggctgtcaaccgaaaccgggttcgcactgctggggggacacccgtgcttcctgaccacccaagacatccacctgggagtgaacgaatcgctgaccgacaccgcccgcgtgctgagctcaatggcggacgccgtgctggcccgcgtgtacaagcagtccgacctggacaccctggccaaggaagcgtccatcccgatcatcaacggactgtccgacctgtaccacccgatccagatcctggcagactacctgaccctgcaagaacactacagctccctgaagggcctgaccctgtcatggatcggggacgggaacaacatcctgcactccataatgatgtcagccgccaagttcggaatgcacctccaagccgcaaccccgaagggctacgaaccggacgcatcagtgaccaaactggccgagcagtacgccaaggaaaacggcaccaagctcctgctgaccaacgacccgctggaggccgcacacggggggaacgtgctgatcaccgacacctggatctccatgggacaggaggaggaaaagaagaagcggctgcaggcgttccaggggtaccaggtcaccatgaaaaccgcgaaggtcgcggcatcagactggaccttcctgcactgcctgccccggaagccggaagaggtggacgacgaggtgttctactcgccgcgctcgctggtgttccccgaggcggagaacaggaagtggaccatcatggcggtgatggtcagcctcctgaccgactactcgccgcagctgcagaagccgaagttctgataactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 98 - RNA, 1471 nt, synthetic construct
  misc_feature 1..1471: note = Synthetic construct
  source 1..1471: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctggtcaacctccgcatcctcctcaacaacgccgcattcagaaacgggcacaacttcatggtcagaaacttccgctgcgggcaacccctacaaaaccgggtccagctcaaggggcgggacctcctgaccctgaagaacttcaccggcgaagagatcaagtacatgctgtggctctccgccgacctgaagttccgcatcaagcagaagggagagtacctcccgctgctgcaagggaagtcgctggggatgatcttcgagaagcggtcaaccagaacccggctgtcaaccgaaaccgggttcgcactgctggggggacacccgtgcttcctgaccacccaagacatccacctgggagtgaacgaatcgctgaccgacaccgcccgcgtgctgagctcaatggcggacgccgtgctggcccgcgtgtacaagcagtccgacctggacaccctggccaaggaagcgtccatcccgatcatcaacggactgtccgacctgtaccacccgatccagatcctggcagactacctgaccctgcaagaacactacagctccctgaagggcctgaccctgtcatggatcggggacgggaacaacatcctgcactccataatgatgtcagccgccaagttcggaatgcacctccaagccgcaaccccgaagggctacgaaccggacgcatcagtgaccaaactggccgagcagtacgccaaggaaaacggcaccaagctcctgctgaccaacgacccgctggaggccgcacacggggggaacgtgctgatcaccgacacctggatctccatgggacaggaggaggaaaagaagaagcggctgcaggcgttccaggggtaccaggtcaccatgaaaaccgcgaaggtcgcggcatcagactggaccttcctgcactgcctgccccggaagccggaagaggtggacgacgaggtgttctactcgccgcgctcgctggtgttccccgaggcggagaacaggaagtggaccatcatggcggtgatggtcagcctcctgaccgactactcgccgcagctgcagaagccgaagttctgataactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 99 - RNA, 1371 nt, synthetic construct
  misc_feature 1..1371: note = Synthetic construct
  source 1..1371: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctggtcaacctccgcatcctcctcaacaacgccgcattcagaaacgggcacaacttcatggtcagaaacttccgctgcgggcaacccctacaaaaccgggtccagctcaaggggcgggacctcctgaccctgaagaacttcaccggcgaagagatccggtacatgctgtggctctccgccgacctgaagttccgcatcaagcagaagggagagtacctcccgctgctgcaagggaagtcgctggggatgatcttcgagaagcggtcaaccagaacccggctgtcaaccgaaaccgggttcgcactgctggggggacacccgtgcttcctgaccacccaagacatccacctgggagtgaacgaatcgctgaccgacaccgcccgcgtgctgagctcaatggcggacgccgtgctggcccgcgtgtacaagcagtccgacctggacaccctggccaaggaagcgtccatcccgatcatcaacggactgtccgacctgtaccacccgatccagatcctggcagactacctgaccctgcaagaacactacagctccctgaagggcctgaccctgtcatggatcggggacgggaacaacatcctgcactccataatgatgtcagccgccaagttcggaatgcacctccaagccgcaaccccgaagggctacgaaccggacgcatcagtgaccaaactggccgagcagtacgccaaggaaaacggcaccaagctcctgctgaccaacgacccgctggaggccgcacacggggggaacgtgctgatcaccgacacctggatctccatgggacaggaggaggaaaagaagaagcggctgcaggcgttccaggggtaccaggtcaccatgaaaaccgcgaaggtcgcggcatcagactggaccttcctgcactgcctgccccggaagccggaagaggtggacgacgaggtgttctactcgccgcgctcgctggtgttccccgaggcggagaacaggaagtggaccatcatggcggtgatggtcagcctcctgaccgactactcgccgcagctgcagaagccgaagttctgataactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 100 - RNA, 1471 nt, synthetic construct
  misc_feature 1..1471: note = Synthetic construct
  source 1..1471: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctggtcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgataactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 101 - RNA, 1371 nt, synthetic construct
  misc_feature 1..1371: note = Synthetic construct
  source 1..1371: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgctggtcaacctgcgcatcctgctgaacaacgccgccttccgcaacggccacaacttcatggtgcgcaacttccgctgcggccagcccctgcagaaccgggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgataactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 102 - RNA, 1368 nt, synthetic construct
  misc_feature 1..1368: note = Synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgcttgtcaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 103 - RNA, 1368 nt, synthetic construct
  misc_feature 1..1368: note = Synthetic construct
  source 1..1368: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgcttgtcaatctccgcatcctccttaacaacgccgcgtttagaaacggccacaacttcatggtccggaacttcagatgtggccagccgcttcaaaaccgggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 104 - RNA, 1371 nt, synthetic construct
  misc_feature 1..1371: note = Synthetic construct
  source 1..1371: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgggccttgtcaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 105 - RNA, 1485 nt, synthetic construct
  misc_feature 1..1485: note = Synthetic construct
  source 1..1485: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgggccttgtcaatctccgcatcctccttaacaacgccgcgtttagaaacggccacaacttcatggtccggaacttcagatgtggccagccgcttcaaaaccgggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 106 - RNA, 1374 nt, synthetic construct
  misc_feature 1..1374: note = Synthetic construct
  source 1..1374: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgggcggacttgtcaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagctcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 107 - RNA, 1377 nt, synthetic construct
  misc_feature 1..1377: note = Synthetic construct
  source 1..1377: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccatggcccttttcaatctccgcatcctccttaacaacgccgcgtttagaaacggccacaacttcatggtccggaacttcagatgtggccagccgcttcaaggcaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagataagtgaactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 108 - RNA, 1377 nt, synthetic construct
  misc_feature 1..1377: note = Synthetic construct
  source 1..1377: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccatggcccttttcaatctccgcatcctccttaacaacgccgcgtttagaaacggccacaacttcatggtccggaacttcagatgtggccagccgcttcaaggccgggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagataagtgaactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 109 - RNA, 1377 nt, synthetic construct
  misc_feature 1..1377: note = Synthetic construct
  source 1..1377: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccatggcccttttcaatctccgcatcctccttaacaacgccgcgtttagaaacggccacaacttcatggtccggaacttcagatgtggccagccgcttcaaggccgggtccagctgaagggccgggatcttctgaccctgaagaactttactggcgaagagatcaggtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagataagtgaactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 110 - RNA, 1377 nt, synthetic construct
  misc_feature 1..1377: note = Synthetic construct
  source 1..1377: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccatggcccttgtcaatctccgcatcctccttaacaacgccgcgtttagaaacggccacaacttcatggtccggaacttcagatgtggccagccgcttcaaggcagggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctaagtgaatagactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 111 - RNA, 1496 nt, synthetic construct
  misc_feature 1..1496: note = Synthetic construct
  source 1..1496: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccatggcccttttcaatctccgcatcctccttaacaacgccgcgtttagaaacggccacaacttcatggtccggaacttcagatgtggccagccgcttcaagtcaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagataagtgaactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 112 - RNA, 1377 nt, synthetic construct
  misc_feature 1..1377: note = Synthetic construct
  source 1..1377: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccatggcccttttcaatctccgcatcctccttaacaacgccgcgtttagaaacggccacaacttcatggtccggaacttcagatgtggccagccgcttcaagtcagggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagataagtgaactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 113 - RNA, 1282 nt, synthetic construct
  misc_feature 1..1282: note = Synthetic construct
  source 1..1282: mol_type = other RNA
ggcagaaaaatttgctacattgtttcacaaacttcaaatattattcatttatttagatctattattacatcaaaacaaaaagccgccaccatgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaggtctctagtaatgagctggagcctcggtagccgttcctcctgcccgctgggcctcccaacgggccctcctcccctccttgcaccggcccttcctggtctttgaataaagtctgagtgggcatctag

SEQ ID NO: 114 - RNA, 1285 nt, synthetic construct
  misc_feature 1..1285: note = Synthetic construct
  source 1..1285: mol_type = other RNA
ggcagaaaaatttgctacattgtttcacaaacttcaaatattattcatttatttagatctattattacatcaaaacaaaaagccgccaccatgggagtattcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaggtctctagtaatgagctggagcctcggtagccgttcctcctgcccgctgggcctcccaacgggccctcctcccctccttgcaccggcccttcctggtctttgaataaagtctgagtgggcatctag

SEQ ID NO: 115 - RNA, 1285 nt, synthetic construct
  misc_feature 1..1285: note = Synthetic construct
  source 1..1285: mol_type = other RNA
ggcagaaaaatttgctacattgtttcacaaacttcaaatattattcatttatttagatctattattacatcaaaacaaaaagccgccaccatgggagtattcaacctgcgcatcctgctgaacaacgccgccttccgcaacggccacaacttcatggtgcgcaacttccgctgcggccagcccctgcagaaccgggtgcagctgaagggccgcgacctgctgaccctgaagaacttcaccggcgaggagatccggtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaggtctctagtaatgagctggagcctcggtagccgttcctcctgcccgctgggcctcccaacgggccctcctcccctccttgcaccggcccttcctggtctttgaataaagtctgagtgggcatctag

SEQ ID NO: 116 - RNA, 1285 nt, synthetic construct
  misc_feature 1..1285: note = Synthetic construct
  source 1..1285: mol_type = other RNA
ggcagaaaaatttgctacattgtttcacaaacttcaaatattattcatttatttagatctattattacatcaaaacaaaaagccgccaccatgctggtattcaacctgcgcatcctgctgaacaacgccgccttccgcaacggccacaacttcatggtgcgcaacttccgctgcggccagcccctgcagaaccgggtgcagctgaagggccgcgacctgctgaccctgaagaacttcaccggcgaggagatccggtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaggtctctagtaatgagctggagcctcggtagccgttcctcctgcccgctgggcctcccaacgggccctcctcccctccttgcaccggcccttcctggtctttgaataaagtctgagtgggcatctag

SEQ ID NO: 117 - RNA, 1225 nt, synthetic construct
  misc_feature 1..1225: note = Synthetic construct
  source 1..1225: mol_type = other RNA
attattacatcaaaacaaaaagccgccaccatgggagtattcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaggtctctagtaatgagctggagcctcggtagccgttcctcctgcccgctgggcctcccaacgggccctcctcccctccttgcaccggcccttcctggtctttgaataaagtctgagtgggcatctag

SEQ ID NO: 118 - RNA, 1225 nt, synthetic construct
  misc_feature 1..1225: note = Synthetic construct
  source 1..1225: mol_type = other RNA
attattacatcaaaacaaaaagccgccaccatgggagtattcaacctgcgcatcctgctgaacaacgccgccttccgcaacggccacaacttcatggtgcgcaacttccgctgcggccagcccctgcagaaccgggtgcagctgaagggccgcgacctgctgaccctgaagaacttcaccggcgaggagatccggtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaggtctctagtaatgagctggagcctcggtagccgttcctcctgcccgctgggcctcccaacgggccctcctcccctccttgcaccggcccttcctggtctttgaataaagtctgagtgggcatctag

SEQ ID NO: 119 - RNA, 1228 nt, synthetic construct
  misc_feature 1..1228: note = Synthetic construct
  source 1..1228: mol_type = other RNA
attattacatcaaaacaaaaagccgccaccatgctggtattcaacctgcgcatcctgctgaacaacgccgccttccgcaacggccacaacttcatggtgcgcaacttccgctgcggccagcccctgcagaaccgggtgcagctgaagggccgcgacctgctgaccctgaagaacttcaccggcgaggagatccggtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaggtctctagtaatgagctggagcctcggtagccgttcctcctgcccgctgggcctcccaacgggccctcctcccctccttgcaccggcccttcctggtctttgaataaagtctgagtgggcagcatctag

SEQ ID NO: 120 - RNA, 1376 nt, synthetic construct
  misc_feature 1..1376: note = Synthetic construct
  source 1..1376: mol_type = other RNA
tcaacacaacatatacaaaacaaacgaatctcaagcaatcaagcattctacttctattgcagcaatttaaatcatttcttttaaagcaaaagcaattttctgaaaattttcaccatttacgaacgatagccaccatgttgttcaacttgaggatcttgttgaacaacgccgccttcaggaacggacacaacttcatggtaaggaacttcaggtgcggacagcccttgcagaacaaagtacagttgaaaggaagggacttgttgacattgaaaaacttcacaggagaagaaatcaaatacatgttgtggttgtcggccgacttgaaattcaggatcaaacagaaaggagaatacttgcccttgttgcagggaaaatcgttgggaatgatcttcgaaaaaaggtcgacaaggacaaggttgtcgacagaaacaggattcgccttgttgggaggacacccctgcttcttgacaacacaggacatccacttgggagtaaacgaatcgttgacagacacagccagggtattgtcgtcgatggccgacgccgtattggccagggtatacaaacagtcggacttggacacattggccaaagaagcctcgatccccatcatcaacggattgtcggacttgtaccaccccatccagatcttggccgactacttgacattgcaggaacactactcgtcgttgaaaggattgacattgtcgtggatcggagacggaaacaacatcttgcactcgatcatgatgtcggccgccaaattcggaatgcacttgcaggccgccacacccaaaggatacgaacccgacgcctcggtaacaaaattggccgaacagtacgccaaagaaaacggaacaaaattgttgttgacaaacgaccccttggaagccgcccacggaggaaacgtattgatcacagacacatggatctcgatgggacaggaagaagaaaaaaaaaaaaggttgcaggccttccagggataccaggtaacaatgaaaacagccaaagtagccgcctcggactggacattcttgcactgcttgcccaggaaacccgaagaagtagacgacgaagtattctactcgcccaggtcgttggtattccccgaagccgaaaacaggaaatggacaatcatggccgtaatggtatcgttgttgacagactactcgccccagttgcagaaacccaaattctgaatagtgaactcgagctagtgactgactaggatctggttaccactaaaccagcctcaagaacacccgaatggagtctctaagctacataataccaacttacacttacaaaatgttgtcccccaaaatgtagccattcgtatctgctcctaataaaaagaaagtttcttcacattctag

SEQ ID NO: 121 - RNA, 1222 nt, synthetic construct
  misc_feature 1..1222: note = Synthetic construct
  source 1..1222: mol_type = other RNA
attattacatcaaaacaaaaagccgccaccatgctgttcaacctgcgcatcctgctgaacaacgccgccttccgcaacggccacaacttcatggtgcgcaacttccgctgcggccagcccctgcagggcaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaggtctctagtaatgagctggagcctcggtagccgttcctcctgcccgctgggcctcccaacgggccctcctcccctccttgcaccggcccttcctggtctttgaataaagtctgagtgggcatctag

SEQ ID NO: 122 - RNA, 1222 nt, synthetic construct
  misc_feature 1..1222: note = Synthetic construct
  source 1..1222: mol_type = other RNA
attattacatcaaaacaaaaagccgccaccatgctgttcaacctgcgcatcctgctgaacaacgccgccttccgcaacggccacaacttcatggtgcgcaacttccgctgcggccagcccctgcagggccgggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaggtctctagtaatgagctggagcctcggtagccgttcctcctgcccgctgggcctcccaacgggccctcctcccctccttgcaccggcccttcctggtctttgaataaagtctgagtgggcatctag

SEQ ID NO: 123 - RNA, 1222 nt, synthetic construct
  misc_feature 1..1222: note = Synthetic construct
  source 1..1222: mol_type = other RNA
attattacatcaaaacaaaaagccgccaccatgctgttcaacctgcgcatcctgctgaacaacgccgccttccgcaacggccacaacttcatggtgcgcaacttccgctgcggccagcccctgcagggccgggtgcagctgaagggccgcgacctgctgaccctgaagaacttcaccggcgaggagatccggtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaggtctctagtaatgagctggagcctcggtagccgttcctcctgcccgctgggcctcccaacgggccctcctcccctccttgcaccggcccttcctggtctttgaataaagtctgagtgggcatctag

SEQ ID NO: 124 - RNA, 1282 nt, synthetic construct
  misc_feature 1..1282: note = Synthetic construct
  source 1..1282: mol_type = other RNA
ggcagaaaaatttgctacattgtttcacaaacttcaaatattattcatttatttagatctattattacatcaaaacaaaaagccgccaccatgctgttcaacctgcgcatcctgctgaacaacgccgccttccgcaacggccacaacttcatggtgcgcaacttccgctgcggccagcccctgcagggcaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaggtctctagtaatgagctggagcctcggtagccgttcctcctgcccgctgggcctcccaacgggccctcctcccctccttgcaccggcccttcctggtctttgaataaagtctgagtgggcatctag

SEQ ID NO: 125 - RNA, 21 nt, synthetic construct
  misc_feature 1..21: note = Synthetic construct
  source 1..21: mol_type = other RNA
cacaaagagtaaagaagaaca

SEQ ID NO: 126 - RNA, 21 nt, synthetic construct
  misc_feature 1..21: note = Synthetic construct
  source 1..21: mol_type = other RNA
aacactaaaagtagaagaaaa

SEQ ID NO: 127 - RNA, 21 nt, synthetic construct
  misc_feature 1..21: note = Synthetic construct
  source 1..21: mol_type = other RNA
ctcagaaagataagatcagcc

SEQ ID NO: 128 - DNA, 1065 bp, synthetic construct; identical to SEQ ID NO: 2
  misc_feature 1..1065: note = Synthetic construct
  source 1..1065: mol_type = other DNA

SEQ ID NO: 129 - DNA, 1065 bp, synthetic construct
  misc_feature 1..1065: note = Synthetic construct
  source 1..1065: mol_type = other DNA
atgctctttaatctgcgcatcttactgaacaacgccgcattccggaacggtcacaacttcatggtccgcaatttccgctgtggccagccgcttcaaaacaaggtccagctgaagggacgggatctgctgacactgaagaacttcaccggagaagagatcaagtacatgctgtggctcagcgcagacttgaagttccggatcaagcagaagggagaatacttgcccctgctgcaaggaaagtcgctgggaatgatttttgagaagcggtcaactcgcaccagactctccaccgaaactggtttcgcactgcttggcgggcacccttgcttcctgacgactcaggacatccacctcggcgtgaacgaatcgctaaccgataccgccagagtgctttcttccatggccgacgcggtgctggccagggtgtacaagcagtccgacctcgataccttggcaaaggaggcttccattcccatcatcaacggcctgagcgacctgtaccacccaatccaaatcctggctgactacctgaccctgcaagagcactacagcagcctgaagggtctgaccctgtcatggattggcgatggaaacaatattctgcactccatcatgatgtccgccgcgaagttcggaatgcatctgcaagccgccactccaaaaggatacgaaccggatgcgtccgtgaccaagttggcggaacagtacgcgaaggagaacggaaccaagcttctgctgactaacgaccccctcgaggctgcgcatgggggcaacgtgctgattaccgacacctggatctccatggggcaggaggaagagaagaagaagagactgcaggcattccaggggtaccaggtcaccatgaaaaccgcaaaagtggcagcttcggactggactttcctgcattgcctgccgaggaagccggaggaagtcgacgacgaagtgttctactcgcctcggtccctggtgttccccgaggccgaaaaccggaagtggaccatcatggccgtgatggtgtccttgctgactgactatagcccgcagctgcagaagcctaagttctag

SEQ ID NO: 130 - DNA, 1065 bp, synthetic construct
  misc_feature 1..1065: note = Synthetic construct
  source 1..1065: mol_type = other DNA
atgctgtttaacctacgtattttgctcaacaatgcagcctttagaaacggacataactttatggttcgaaactttcgctgcgggcagccactgcagaacaaggtccagctgaaagggagagatttgctcacgctgaagaactttactggcgaagaaatcaagtatatgctgtggttgtccgcggacctcaagtttcggattaagcagaaaggggagtatctgccactgctgcaaggaaagagcctcggcatgatcttcgagaagcggagcactcggaccaggctgagtaccgaaactggcttcgcattgttgggtggacatccatgttttctgacaacgcaggacattcatctgggcgtgaacgagagtctgacggacacagctcgcgttctgtcctctatggctgatgcggtgttggcccgggtctataagcagtccgatttggacaccttggctaaggaagctagcataccgattatcaatgggctgtccgacctgtatcaccctattcaaatcctggccgactacctcacactgcaagaacactatagctcattgaagggactgaccctgagctggataggggacggaaacaacatcctacatagcattatgatgtccgctgccaagtttggcatgcatcttcaagccgccacgccaaagggttatgagcccgacgcgtcagtgacaaagctggccgagcagtacgctaaggagaatggtaccaaattactgctgactaatgatccactggaggctgcacatggcggcaatgtactgatcaccgacacatggatctcgatgggccaggaggaagaaaagaagaagaggcttcaggccttccaaggctaccaggtcaccatgaaaacagctaaggttgcagcatctgattggacctttctgcactgtctgccaaggaagcccgaagaggtggacgatgaagtattctatagcccacggagtttggtgttccctgaggctgaaaataggaagtggacaattatggccgtaatggtgtccctgttaaccgactactctccgcaactgcagaaacctaagttttag

SEQ ID NO: 131 - DNA, 1065 bp, synthetic construct
  misc_feature 1..1065: note = Synthetic construct
  source 1..1065: mol_type = other DNA
atgctgtttaacttaaggatcctgctgaacaacgccgcttttcgtaacggtcataactttatggtccggaactttagatgtggccagccgctgcagaacaaggttcagctgaaggggagggatctgctgaccttgaagaactttaccggcgaagagatcaagtacatgttgtggctgagcgccgatctgaagtttaggattaagcagaagggggagtatttgccactgctgcaaggaaaatccttggggatgatcttcgagaagcgctccactagaacccggctaagcacagaaaccggcttcgcacttctgggtggacatccctgttttctgacgacgcaggatatacacctgggcgtgaatgagagtctgacggacacagctagggtgttgagcagcatggccgatgcagtactggcccgcgtttataagcagagcgacttggacacactggccaaggaagcgtcaattccgattatcaatgggctgtcagacctgtatcatcccattcaaatcttggctgactatctgaccctgcaagaacattacagctccctgaagggcctcacgttgtcctggattggcgacggaaacaacattctgcattcgatcatgatgagcgctgctaagtttggcatgcacctccaagccgctacacctaagggatatgagcctgatgccagcgtaaccaagctggccgaacagtacgcgaaggagaatggcacgaaactgctgttgacaaatgacccactggaggcagctcacggtggcaacgtgctgatcaccgacacgtggatatctatgggacaggaagaagagaagaagaagcggctgcaggcattccaagggtatcaggtcaccatgaaaacggccaaggttgctgcatccgactggacatttctgcattgcttgccccgcaaaccagaagaagtagacgacgaagtcttttattccccacggtcgctggtgttccccgaggcggagaatcgaaagtggacgattatggccgtgatggtgtccctgctgactgattactctccccaactgcaaaagcctaagttttag

SEQ ID NO: 132 - DNA, 1065 bp, synthetic construct
  misc_feature 1..1065: note = Synthetic construct
  source 1..1065: mol_type = other DNA
atgcttttcaacctgaggatcctcctgaacaacgccgcctttcgcaatggtcacaactttatggtccggaacttcagatgcggccagccgctgcagaacaaggtccagctgaagggacgggatctgctgactctgaagaacttcaccggagaagagatcaagtacatgctgtggctgtcggccgacctgaagttcaggatcaagcagaagggagaatacctcccgctgctgcaaggaaagtccctgggcatgattttcgagaagcgctcgaccagaactcggttgtccaccgaaaccgggtttgcgctgctgggcggacatccttgcttcctgacgactcaggatattcacctgggagtgaacgagtcgctgaccgacaccgccagagtgctgagctcgatggccgacgccgtgttggcacgcgtgtacaagcagtccgatctggataccctggccaaagaagcttccatcccgatcattaacgggctgagcgacctctaccaccccattcaaatcctggccgactacctgactctgcaagaacactacagctcgctgaaggggttgactctgtcctggatcggcgacggaaacaacatcctgcactccatcatgatgtcggccgcaaagttcggcatgcatttgcaagccgccaccccaaagggctacgaaccagacgcgagcgtcaccaagctggccgaacagtacgcgaaggaaaatggtactaagctgctgctgaccaacgacccattggaagctgcccatggtggaaacgtgctgatcaccgacacctggatctcgatgggccaggaagaggagaagaagaagcggctgcaggcgttccaggggtatcaggtcaccatgaaaacagccaaagtggcagcgtcagactggaccttcctccactgtctgcctcgcaagccagaggaggtggacgacgaggtgttctactcccctcggtccctcgtgttccctgaggctgagaaccggaagtggaccattatggccgtgatggtgtcactcctgactgattactccccgcaactgcagaagcccaagttctag

SEQ ID NO: 133 - DNA, 1065 bp, synthetic construct
  misc_feature 1..1065: note = Synthetic construct
  source 1..1065: mol_type = other DNA
atgctgtttaacctgaggatcctattgaacaatgctgcttttcgtaatggccataactttatggttcggaactttagatgcgggcagccactgcagaacaaggtccagttgaaaggccgcgatctgttgacattgaagaactttaccggcgaagagattaagtatatgctgtggctgtctgctgacctcaagtttcgaatcaagcagaagggcgaatatctccccctgctgcaaggaaagtctctcggcatgatctttgagaagcggagtacccgaacacggctgagcaccgaaacgggcttcgcactgctggggggccatccctgttttctgacaacgcaggacatccacttgggggttaacgaatcattgactgataccgcccgcgtactgtcatccatggccgacgctgtgctggctagggtgtacaagcagtcagatctggatacactggccaaggaagctagcataccaatcatcaatggactgagtgacctttatcacccgattcaaatactagccgattatctgaccctgcaagagcattactcctcgctgaaaggcctcacgctgtcctggatcggcgacggcaacaacattctgcatagtattatgatgtctgctgccaaattcggcatgcatctgcaagctgctacgccgaagggttatgaacccgacgcgtcagttacgaagctcgctgagcagtatgcaaaggagaatggcacaaagctgttgcttaccaacgatcccctggaagctgctcatggcggcaatgtgctgattactgacacctggatttcaatgggccaggaggaggagaagaagaagaggttacaggcttttcaaggttaccaagtcacgatgaaaaccgctaaggtcgcagccagcgactggacattcctgcactgtctgccaagaaagccggaagaagtggacgacgaggtgttctattccccgcggtctttggtgtttccggaggccgaaaacaggaaatggaccattatggccgtgatggtatcgttgctgacggactacagccctcagttgcaaaagcccaagttctag

SEQ ID NO: 134 - DNA, 1065 bp, synthetic construct
  misc_feature 1..1065: note = Synthetic construct
  source 1..1065: mol_type = other DNA
atgctctttaacctccgcatcctcctcaacaacgccgccttccggaatgggcataacttcatggtccggaacttcagatgcggccagcccctgcaaaacaaggtccagttgaagggacgggacctccttacgctgaagaactttaccggagaagagattaagtacatgctgtggttgtccgctgacctcaagttccgcattaagcagaagggagaatatctgccgctgctgcaaggaaagagcctgggcatgatcttcgaaaagcgctccactagaacccggctgtcgactgagactggattcgccttgctcggtggacacccgtgcttcctgacgacccaggacatccacctgggagtgaacgagtcacttacggataccgcgagggtgctgtcctcaatggccgacgcagtgctcgcgcgcgtgtacaagcagtcagatctggataccctggccaaggaagccagcattcccatcatcaacggactgagcgacctttaccacccaatccagatcctcgccgactacttaaccctgcaagagcactacagctccctgaagggactgactctgtcctggatcggggatggaaacaacatcctgcactccatcatgatgtctgccgctaagtttgggatgcatctgcaagccgcaacccctaagggatacgagcccgacgcctcggtgaccaaacttgcggaacagtacgccaaggaaaacggtaccaagctgctgctgaccaacgaccctctggaagcggcccacggaggaaatgtgctgattaccgacacctggatttcgatgggccaggaggaggagaagaagaagagactgcaggcgttccagggatatcaggtcaccatgaaaaccgccaaggtcgccgccagcgactggaccttcctgcactgtctccctcggaaaccggaagaagtggatgacgaggtgttctactccccgcgctcgctggtgttcccggaggctgaaaacaggaagtggacaatcatggccgtgatggtgtccctgttgaccgactactccccacaactgcagaagcccaagttctag

SEQ ID NO: 135 - DNA, 1065 bp, synthetic construct
  misc_feature 1..1065: note = Synthetic construct
  source 1..1065: mol_type = other DNA
atgcttttcaatctgcgcatcctcctgaacaacgccgccttccgcaatggacacaactttatggtccgcaacttccgctgtgggcagccgctgcagaacaaggtccagctcaaggggagagatctcctgaccctgaagaacttcactggagaggagatcaagtacatgctgtggctgtccgccgacctgaaatttcggattaagcagaagggcgaatacctcccactgctgcaaggaaagtctttgggcatgatcttcgaaaagagaagcacccggacccggttgagcaccgaaactgggttcgcgctcctcggtggacacccgtgcttcctgaccacccaagatattcatctgggtgtcaacgaaagcctgaccgacaccgccagggtgctgtcatccatggctgacgcagtgctcgcccgggtgtacaagcagtcagacctggacaccctcgccaaggaagcttcgatccctatcatcaacggactttccgacctgtaccaccccatccaaattctggccgactacctgactctgcaagaacactatagctcgctgaaaggacttactctgtcctggatcggggacggcaacaacattctccattccatcatgatgtccgctgccaagttcggaatgcaccttcaagcagcgactcccaagggatacgaacctgatgcctccgtgactaagctggcagagcagtacgccaaggagaacggtacaaagctgctgctcacgaacgaccccctggaggcggcccacggcggaaacgtgctgattaccgatacctggatctcaatgggccaggaagaggagaagaagaagcggctccaggcgtttcaaggctaccaggtcaccatgaaaaccgcgaaggtcgccgcctccgactggactttcttgcactgcctgccgcggaagcccgaggaagtggatgacgaagtgttctactcgccgagatcgttggtgttccctgaggccgaaaacaggaagtggaccatcatggccgtgatggtgtccctgctgactgattacagcccacagctgcagaagcctaagttctag

SEQ ID NO: 136 - DNA, 1065 bp, synthetic construct
  misc_feature 1..1065: note = Synthetic construct
  source 1..1065: mol_type = other DNA
atgcttttcaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctag

SEQ ID NO: 137 - DNA, 1065 bp, synthetic construct
  misc_feature 1..1065: note = Synthetic construct
  source 1..1065: mol_type = other DNA
atgcttttcaacctgagaatcctcctgaacaacgccgccttccgcaatggtcataacttcatggtccgcaactttcgctgcggacagcctctccaaaacaaggtccagctcaaggggcgcgacctcctcacactgaagaacttcactggagaagaaatcaagtacatgctgtggctgagcgccgatctgaagttccggatcaagcagaagggagagtaccttcctctgctgcaagggaagtccttgggaatgattttcgagaagcggtccacccggaccaggctgagcactgaaactggcttcgccctgctgggaggccacccttgtttcctgaccactcaggacatccacctgggcgtgaacgagtccctgaccgatactgccagagtgctgtcctccatggccgacgccgtgctcgcccgggtgtacaagcagtcagacctcgatacgctggccaaggaagcctccattcccattatcaatggtctgtcggacctctaccatccaatccaaatcctcgccgactacctgactctgcaagaacactacagctcactcaagggcctcaccctctcctggatcggcgacggaaacaacatccttcactcgattatgatgtcggccgcgaagttcgggatgcacctccaagctgccactccaaaaggctacgagccggatgcctcagtgactaagttggcggaacagtatgcgaaggagaacggtaccaagctcctgctgactaacgacccgctggaggccgcccacgggggaaacgtgctcatcaccgatacttggatttccatgggacaggaggaagagaagaagaagcggttgcaggcatttcagggctaccaggtcaccatgaaaactgccaaagtcgccgccagcgactggaccttcctgcactgcctgccccgcaagcctgaagaagtggacgacgaggtgttctactctccccggtccctcgtgttccctgaggccgaaaacaggaagtggaccatcatggctgtgatggtgtccctcctgaccgactacagccctcagctccaaaaacccaagttttag

SEQ ID NO: 138 - DNA, 1065 bp, synthetic construct
  misc_feature 1..1065: note = Synthetic construct
  source 1..1065: mol_type = other DNA
atgcttttcaacctgagaatcctcttgaacaatgctgcttttcggaatggccacaactttatggttcggaacttccgttgcggccagcctttacaaaacaaggtccagctgaagggccgggatttgctcacactgaagaactttactggggaggagattaagtatatgctgtggctgtccgctgacctgaagtttaggatcaagcagaagggcgaatatctgccgctgctgcaagggaaaagtctgggcatgatttttgaaaagcgctctacccggaccagactgtctacggaaacaggctttgccctgctgggcggccacccctgttttctgacaacgcaggacatccatctgggcgtgaacgaatcactgaccgatactgctcgggtactcagttctatggctgacgcagtgctggctagggtgtacaagcagagcgacttggacacactggctaaggaggccagcatccccattatcaatggcctgtctgatttgtaccatcccattcaaatcctggctgattatctgacactacaagagcattactcaagtctgaagggtttgactctctcctggatcggcgacggcaacaacattttacattccattatgatgagtgctgctaagtttggcatgcatttgcaagctgctaccccaaagggctatgaacctgacgctagcgtaaccaagttggccgaacagtatgctaaagagaatggcaccaagctgctcctgacgaatgaccccctggaagctgctcatggcggaaacgtacttataactgatacatggattagcatgggccaggaagaggagaagaagaagagactgcaggccttccaaggctatcaggtcaccatgaaaactgccaaggttgcagctagcgactggaccttcctgcactgtttgccgaggaaacccgaggaggtggacgatgaagtcttttattctccccgctccttggtgtttcccgaggctgaaaatcgaaagtggacgataatggcagtgatggtgtccctactgaccgactattctccacaactgcagaagcctaaattctag

SEQ ID NO: 139 - DNA, 1065 bp, synthetic construct
  misc_feature 1..1065: note = Synthetic construct
  source 1..1065: mol_type = other DNA
atgcttttcaatctgaggatcctgctgaacaacgctgcttttcgcaacggtcataactttatggttcgcaattttcgttgtggccagccgctgcagaacaaggttcagctgaagggcagagatctgctgactctgaagaacttcactggggaagaaatcaagtatatgttatggctgtccgcggatctgaaatttcgaatcaagcagaagggcgaatatcttcccctgctgcaagggaaatccttgggcatgatttttgagaagaggagcactaggactagattgtcaacagaaacaggctttgctttgttgggcggacatccctgctttctgacgacacaggatatccacctcggcgtaaacgagtccctcaccgacactgctagggtactgagcagcatggccgacgctgtgctagcccgggtttacaagcagtcagacctggacacccttgccaaggaagcttctattccaattatcaacggcctgagtgacctgtatcaccctattcaaatactcgccgactatttgacgcttcaagaacattacagcagcctcaagggcttaaccttgagttggataggcgacggcaacaatatcctgcattccattatgatgtctgccgctaagtttggcatgcatctacaagccgcaacacccaagggctatgaacccgacgctagcgtgaccaagctggccgagcagtatgctaaggaaaatggcacaaagctccttcttaccaacgatcccctggaggctgctcacggcggcaacgtgctgattaccgatacatggattagcatgggccaggaggaggagaaaaagaagcggctccaggcttttcaaggctatcaggtcaccatgaaaactgcaaaggtcgctgcctccgactggactttcctgcattgtctaccccgcaagcctgaggaagtggacgatgaggtgttctactccccacggagtctggtgttcccggaagcagagaatcggaagtggaccatcatggctgtcatggtgtcgctcttgactgactattctccccaactgcaaaaacccaagttttag

SEQ ID NO: 140 - DNA, 1065 bp, synthetic construct; identical to SEQ ID NO: 139
  misc_feature 1..1065: note = Synthetic construct
  source 1..1065: mol_type = other DNA

SEQ ID NO: 141 - DNA, 1065 bp, synthetic construct
  misc_feature 1..1065: note = Synthetic construct
  source 1..1065: mol_type = other DNA
atgctttttaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcccatcatcaacggcctgagcgacctgtaccacccaatccaaatcctggctgactacctgaccctgcaagagcactacagcagcctgaagggtctgaccctgtcatggattggcgatggaaacaatattctgcactccatcatgatgtccgccgcgaagttcggaatgcatctgcaagccgccacgccaaaaggatacgaaccggatgcgtccgtgacgaagttggcggaacagtacgcgaaggagaacggaaccaagcttctgctgactaacgaccccctcgaggctgcgcatgggggcaacgtgctgattaccgacacctggatctccatggggcaggaggaagagaagaagaagagactgcaggcattccaggggtaccaggtcaccatgaaaaccgcaaaagtggcagcttcggactggactttcctgcattgcctgccgaggaagccggaggaagtcgacgacgaagtgttctactcgcctcggtccctggtgttccccgaggccgaaaaccggaagtggaccatcatggccgtgatggtgtccttgctgactgactatagcccgcagctgcagaagcctaagttctag

SEQ ID NO: 142 - DNA, 1065 bp, synthetic construct
  misc_feature 1..1065: note = Synthetic construct
  source 1..1065: mol_type = other DNA
atgcttttcaacctgagaatcctcttgaacaatgctgcttttcggaatggccacaactttatggttcggaacttccgttgcggccagcctttacaaaacaaggtccagctgaagggccgggatttgctcacactgaagaactttactggagaagagatcaagtacatgctgtggctgtcggccgacctgaagttcaggatcaagcagaagggagaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctcgataccttggcaaaggaggcttccattcccatcatcaacggcctgagcgacctgtaccacccaatccaaatcctggctgactacctgaccctgcaagagcactacagcagcctgaagggtctgaccctgtcatggattggcgatggaaacaatattctgcactccatcatgatgtccgccgcgaagttcggaatgcatctgcaagccgccactccaaaaggatacgaaccggatgcgtccgtgaccaagttggcggaacagtacgcgaaggagaacggaaccaagcttctgctgactaacgaccccctcgaggctgcgcatgggggcaacgtgctgattaccgacacctggatctccatggggcaggaggaagagaagaagaagagactgcaggcattccaggggtaccaggtcaccatgaaaaccgcaaaagtggcagcttcggactggactttcctgcattgcctgccgaggaagccggaggaagtcgacgacgaagtgttctactcgcctcggtccctggtgttccccgaggccgaaaaccggaagtggaccatcatggccgtgatggtgtccttgctgactgactatagcccgcagctgcagaagcctaagttctag

SEQ ID NO: 143 - DNA, 1065 bp, synthetic construct
  misc_feature 1..1065: note = Synthetic construct
  source 1..1065: mol_type = other DNA
atgcttttcaacctgagaatcctcttgaacaatgctgcttttcggaatggccacaactttatggttcggaacttccgttgcggccagcctttacaaaacaaggtccagctgaagggccgggatttgctcacactaaagaactttactggagaagagatcaagtacatgctatggctgtcggccgacctgaagttccgtatcaagcagaagggagaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctcgataccttggcaaaggaggcttccattcccatcatcaacggcctgagcgacctgtaccacccaatccaaatcctggctgactacctgaccctgcaagagcactacagcagcctgaagggtctgaccctgtcatggattggcgatggaaacaatattctgcactccatcatgatgtccgccgcgaagttcggaatgcatctgcaagccgccactccaaaaggatacgaaccggatgcatccgtgaccaagttggcggaacagtacgcgaaggagaacggaaccaagctcctgctgactaacgacccgctcgaggctgcgcatgggggtaacgtgctgattacggacacctggatctccatggggcaggaggaagagaagaagaagagactgcaggcattccaggggtaccaggtcaccatgaaaaccgcaaaagtggcagcttcggactggactttcctgcattgcctgccgaggaagccggaggaagtcgacgacgaagtgttctactcgcctcggtccctggtgttccccgaggccgaaaaccggaagtggaccatcatggccgtgatggtgtccttgctgactgactatagcccgcagctgcagaagcctaagttctag

SEQ ID NO: 144 - DNA, 1065 bp, synthetic construct
  misc_feature 1..1065: note = Synthetic construct
  source 1..1065: mol_type = other DNA
atgcttttcaacctgagaatcctcttgaacaatgctgcttttcggaatggccacaactttatggttcggaacttccgttgcggccagcctttacaaaacaaggtccagctgaagggccgggatttgctcacactaaagaactttactggagaagagatcaagtacatgctatggctgtcggccgacctgaagttccgtatcaagcagaagggagaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctcgataccttggcaaaggaggcttccattcccatcatcaacggcctgagcgacctgtaccacccaatccaaatcctggctgactacctgaccctgcaagagcactacagcagcctgaagggtctgaccctgtcatggattggcgatggaaacaatattctgcactccatcatgatgtccgccgcgaagttcggaatgcatctgcaagccgccactccaaaaggatacgaaccggatgcgtccgtgaccaagttggcggaacagtacgcgaaggagaacggaaccaagcttctgctgactaacgaccccctcgaggctgcgcatgggggcaacgtgctgattaccgacacctggatctccatggggcaggaggaagagaagaagaagagactgcaggcattccaggggtaccaggtcaccatgaaaaccgcaaaagtggcagcttcggactggactttcctgcattgcctgccgaggaagccggaggaagtcgacgacgaagtgttctactcgcctcggtccctggtgttccccgaggccgaaaaccggaagtggaccatcatggccgtgatggtgtccttgctgactgactatagcccgcagctgcagaagcctaagttctag

SEQ ID NO: 145 - DNA, 1065 bp, synthetic construct
  misc_feature 1..1065: note = Synthetic construct
  source 1..1065: mol_type = other DNA
atgctgttcaacctgcgaatcctgctgaacaacgccgcttttcggaacgggcacaactttatggtgaggaactttcgctgcggacagcccctccagaataaggtccagctgaagggcagggacctgctgaccctgaaaaatttcacaggggaggaaatcaagtatatgctgtggctgtcagctgatctgaagttccggatcaagcagaagggcgaatatctgcctctgctccagggcaaaagcctggggatgatcttcgaaaagcgcagtactcggaccagactgtcaaccgagactggattcgctctgctgggaggacacccttgttttctgaccactcaggacattcacctgggagtgaacgagtccctgaccgacactgctcgcgtcctgagctctatggccgacgctgtgctggctcgagtctacaaacagtccgacctggataccctggccaaggaagcttctatcccaattattaacggcctgtcagacctgtatcaccccatccagattctggccgattacctgaccctccaggagcactattctagtctgaaagggctgacactgagttggattggggacggaaacaatatcctgcactctattatgatgtcagccgccaagtttggaatgcacctccaggctgcaaccccaaaaggctacgaacccgatgcctcagtgacaaagctggctgaacagtacgccaaagagaacggcactaagctgctgctgaccaacgaccctctggaggccgctcacggaggcaacgtgctgatcaccgatacctggattagtatgggacaggaggaagagaagaagaagcggctccaggccttccagggctaccaggtgacaatgaaaaccgctaaggtcgcagccagcgattggacctttctgcactgcctgcccagaaagcccgaagaggtggacgacgaggtcttctactctcccagaagcctggtgtttcccgaagctgagaataggaagtggacaattatggcagtgatggtcagcctgctgactgattattcacctcagctccagaaaccaaagttctga

SEQ ID NO: 146 - DNA, 1065 bp, synthetic construct
  misc_feature 1..1065: note = Synthetic construct
  source 1..1065: mol_type = other DNA
atgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcctggccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctga

SEQ ID NO: 147 - DNA, 1065 bp, synthetic construct
  misc_feature 1..1065: note = Synthetic construct
  source 1..1065: mol_type = other DNA
atgctgttcaacctgcgaatcctgctgaacaacgccgcttttcggaacgggcacaactttatggtgaggaactttcgctgcggacagcccctccagaataaggtccagctgaagggcagggacctgctgaccctgaaaaatttcacaggggaggaaatcaagtatatgctgtggctgtcagctgatctgaagttccggatcaagcagaagggcgaatatctgcctctgctccagggcaaaagcctggggatgatcttcgaaaagcgcagtactcggaccagactgtcaaccgagactggattcgctctgctgggaggacacccttgttttctgaccactcaggacattcacctgggagtgaacgagtccctgaccgacactgctcgcgtcctgagctctatggccgacgctgtgctagctcgagtctacaaacagtccgacctggataccctggccaaggaagcttctatcccaattattaacggcctgtcagacctgtatcaccccatccagattctggccgattacctgaccctccaggagcactattctagtctgaaagggctgacactgagttggattggggacggaaacaatatcctgcactctattatgatgtcagccgccaagtttggaatgcacctccaggctgcaaccccaaaaggctacgaacccgatgcctcagtgacaaagctggctgaacagtacgccaaagagaacggcactaagctgctgctgaccaacgaccctctggaggccgctcacggaggcaacgtgctgatcaccgatacctggattagtatgggacaggaggaagagaagaagaagcggctccaggccttccagggctaccaggtgacaatgaaaaccgctaaggtcgcagccagcgattggacctttctgcactgcctgcccagaaagcccgaagaggtggacgacgaggtcttctactctcccagaagcctggtgtttcccgaagctgagaataggaagtggacaattatggcagtgatggtcagcctgctgactgattattcacctcagctccagaaaccaaagttctga

SEQ ID NO: 148 - DNA, 1065 bp, synthetic construct
  misc_feature 1..1065: note = Synthetic construct
  source 1..1065: mol_type = other DNA
atgctgttcaacctgcgaatcctgctgaacaatgccgcttttcggaacgggcacaatttcatggtgaggaactttcgctgcggacagcccctccagaacaaggtccagctgaagggcagggacctgctgaccctgaaaaatttcacaggggaggaaatcaagtacatgctgtggctgtcagccgatctgaagttccggatcaagcagaagggcgaatatctgcctctgctccagggcaaaagcctggggatgatcttcgaaaagcgcagtactcggaccagactgtcaacagagactggattcgcactgctgggaggacacccatgttttctgaccacacaggacattcatctgggagtgaacgagtccctgaccgacacagcacgcgtcctgagctccatggctgatgcagtgctggctcgagtctacaaacagtctgacctggataccctggccaaggaagcttctatcccaatcattaatggcctgagtgacctgtatcaccccatccagattctggccgattacctgaccctccaggagcattattctagtctgaaagggctgacactgagctggattggggacggaaacaatatcctgcactccattatgatgagcgccgccaagtttggaatgcacctccaggctgcaaccccaaaaggctacgaacccgatgcctccgtgacaaagctggcagaacagtatgccaaagagaacggcactaagctgctgctgaccaatgaccctctggaggccgctcacggaggcaacgtgctgatcactgatacctggattagtatgggacaggaggaagagaagaagaagcggctccaggccttccagggctaccaggtgacaatgaaaactgctaaggtcgcagccagcgactggacctttctgcattgcctgcccagaaagcctgaagaggtggacgatgaggtcttctactcacccagaagcctggtgtttcctgaagctgagaataggaagtggacaatcatggcagtgatggtcagcctgctgactgattattcccctcagctccagaaaccaaagttctga

SEQ ID NO: 149 - DNA, 1065 bp, synthetic construct
  misc_feature 1..1065: note = Synthetic construct
  source 1..1065: mol_type = other DNA
atgcttttcaaccttcgcattctcctcaacaacgccgcgtttagaaacggacacaacttcatggtccgcaacttccgctgcggacagccgctgcagaacaaggtccagctcaagggtcgggatctcctgacgctgaagaactttaccggcgaagagattaagtacatgctgtggctgtccgccgaccttaagttccggatcaagcagaagggcgaataccttcccctgctgcaaggaaagtccctgggcatgatcttcgagaagcgcagtaccagaaccagactctccactgaaaccgggttcgcgctgcttggcggccacccgtgtttcctcactacgcaagacatccatcttggcgtgaacgagtcccttaccgacaccgccagggtgctgtcaagcatggccgacgccgtccttgcgcgcgtgtacaagcagtcagaccttgatactctggccaaggaagcctccatccctattatcaacggcctatccgacctttaccacccgatccagatcctcgctgactacctgaccctgcaagaacactacagcagcctcaagggactgactctgtcctggatcggcgacgggaacaacatcctgcactcaatcatgatgagcgcagccaagttcggcatgcatctccaagccgctacacccaagggttatgaaccggacgcctctgtgaccaagttggcagaacagtacgccaaggagaacggtactaagctccttttaaccaacgaccccctcgaagcagcccatggcgggaatgtgctcattaccgatacctggatttcgatgggccaggaggaggagaagaagaagcggctgcaggcgttccagggctaccaggtcaccatgaaaactgccaaagtggccgcctcggattggacctttctccactgcctgcctcggaagcctgaggaggtggacgacgaagtgttctactccccacggtccctcgtgttccccgaggccgaaaataggaagtggaccatcatggccgtgatggtgtccctcttgaccgattacagcccgcagcttcagaagcctaaattctag

SEQ ID NO: 150 - DNA, 1065 bp, synthetic construct
  misc_feature 1..1065: note = Synthetic construct
  source 1..1065: mol_type = other DNA
atgcttttcaatcttcgcatcctgttgaacaacgccgccttccgcaatggtcacaacttcatggtccggaacttcagatgtggacagcctctccaaaacaaggtccagctgaagggaagggacctcttaaccctcaaaaactttactggagaggagatcaagtacatgctgtggcttagcgccgaccttaagttccggatcaagcagaagggagagtacctcccgctgctgcaaggaaagagtcttggaatgatcttcgagaagcggtccaccagaactcgcctctccactgaaaccggattcgcactcctgggtggacacccgtgctttctgaccacccaagacatccacctcggagtgaacgagagcctcacggacaccgcgagagtgctgtcatccatggccgacgccgtgcttgcacgggtctacaagcagtccgatctggacactcttgccaaggaagcctccattcctatcattaacggtctgtcggatctgtaccacccgattcagatccttgcggactacctcacacttcaagaacactattcaagcctaaagggtctgaccctgtcctggatcggagatggaaacaacattctccattccatcatgatgagcgctgccaagttcggaatgcatctccaagcagcgactcctaagggttacgagccggacgcctcagtgactaagctggccgagcagtacgccaaggagaacggtaccaaactgttgcttactaacgacccgcttgaagcggcccatggaggaaacgtgctgattaccgacacctggatttcgatgggacaggaagaggagaagaagaagcggctccaggcgttccagggataccaggtcaccatgaaaacggccaaagtggccgctagcgattggacctttctgcactgcctcccgcgcaagcctgaagaagtggacgacgaagtgttctactcccctcgctctcttgtgttcccggaagccgaaaacaggaagtggaccatcatggccgtgatggtgtccctcctgaccgattacagcccgcagctgcagaagcctaagttctag

SEQ ID NO: 151 - DNA, 1065 bp, synthetic construct
  misc_feature 1..1065: note = Synthetic construct
  source 1..1065: mol_type = other DNA
atgcttttcaatctccgcatcctcctcaacaacgccgcgtttagaaacggccacaacttcatggtccggaacttcagatgtggccagccgcttcagaacaaggtccagctcaagggccgggatcttctgaccctgaagaactttactggcgaagaaatcaagtacatgctctggctctccgccgacttgaagttccgcattaagcagaagggggaataccttccgctgctgcaaggaaagtcgctcggcatgatctttgagaagcgctcaacccgcaccaggctgtccactgaaaccgggttcgcgctgcttggtggccacccctgcttcctgaccacccaagacattcacctcggagtgaacgaatcgctcactgatactgcccgggtgctgtcgtcgatggccgatgcagtgctggccagggtgtacaaacagtccgatctggacactctggccaaggaggcgtccatccctattatcaacggcctttccgacctctaccacccgattcagatccttgccgattacctcaccctgcaagaacactactcgtcactgaagggtctgaccttgtcctggatcggcgacggcaacaacatcctccattccattatgatgtccgccgccaaattcggcatgcatcttcaagccgcaacccctaagggttacgagccggacgcttccgtgaccaagctcgccgagcagtacgctaaggagaacggaaccaagcttctgctgactaacgaccccctagaggcagcccacgggggcaacgtgcttattactgacacctggatctccatgggacaggaagaagagaagaagaagcggttacaggcgttccagggctatcaggtcaccatgaaaaccgccaaggtcgctgcctcggactggaccttcctgcattgcctgcctcgcaagcccgaagaagtggacgacgaggtgttctactcgccacggtcccttgtgttccctgaggccgagaatagaaagtggaccattatggccgtgatggtgtcccttctcaccgactactcgccgcaactgcagaaacccaagttctag

SEQ ID NO: 152 - DNA, 1065 bp, synthetic construct
  misc_feature 1..1065: note = Synthetic construct
  source 1..1065: mol_type = other DNA
atgcttttcaatcttcgcatcctcctcaacaacgccgccttccggaacggtcacaacttcatggtccggaacttccgctgcggccagccgctccaaaacaaagtgcagcttaagggccgcgatctcctgaccctgaagaacttcaccggagaggaaatcaagtacatgctgtggctctcggcggacctgaagtttaggattaagcagaagggggagtatctgccgctgctccaagggaagtcccttggcatgatcttcgaaaagaggtccacccggactcggctcagcaccgaaacaggttttgcacttctggggggccacccgtgcttcctgacgacccaggacatccatctgggtgtcaacgagagtttgaccgacactgccagagtgctgtcatccatggcggacgcggtgctcgcgagagtgtacaagcagtccgatcttgacaccctggcaaaagaggcttcaatcccgatcattaacggactctcggatctgtaccaccctatccaaatcttggccgactacctgaccctgcaagaacactacagctccctgaagggcctgactctttcctggattggcgatggaaacaacattctccattctattatgatgtccgccgccaagttcggcatgcaccttcaagccgccaccccgaagggctacgaacctgacgcctccgtgactaagctagccgaacagtacgctaaggagaacggcactaagcttctccttaccaacgatccgctggaggcggcccatggcggaaatgtgcttatcaccgacacctggattagcatggggcaggaagaagagaagaagaaacggctccaggcattccagggctaccaggtcaccatgaaaactgccaaggtcgccgctagcgactggaccttcctccactgtctgcctcgcaagcctgaagaagtggacgacgaggtgttctactccccgcgctccctcgtgtttcctgaggccgagaacagaaagtggaccatcatggccgtgatggtgtcattacttacggactacagcccgcagctgcagaagccgaagttctag

SEQ ID NO: 153 - DNA, 1065 bp, synthetic construct
  misc_feature 1..1065: note = Synthetic construct
  source 1..1065: mol_type = other DNA
atgctttttaacttgagaatccttctgaacaacgccgctttccgcaacggtcataacttcatggtccggaacttcagatgtggccagcccctccaaaacaaagtgcagctgaagggccgggaccttcttacgctgaagaatttcaccggcgaagaaatcaagtacatgctctggctgtccgccgatcttaagttccgcattaagcagaagggggaatacctcccgctgctgcaagggaagtcgctgggcatgatttttgagaagcggtcaactcgcacccgcctgtccactgaaactggattcgcactgctcggtggccatccctgcttcctgaccacccaagacatccacctcggcgtgaacgagtccctgactgacaccgcccgggtcttatcctcgatggccgatgctgtgcttgcgagggtgtacaagcagtccgacctcgacacactcgcgaaggaggcctccatccccatcatcaacggcctgtccgacctttaccacccaattcagatcctcgccgattacctgaccctgcaagagcactactcgtcgctcaaggggcttaccctctcgtggattggcgacggcaacaacatccttcactccatcatgatgtcggcagcgaagttcggcatgcatctgcaagccgccacgcctaagggttatgaaccggatgcctcagtgaccaagctcgccgaacagtacgcgaaagagaatggaaccaagctacttctgaccaacgaccccctggaggccgctcacggcggcaacgtcctcattaccgatacttggatttcgatgggacaggaagaggaaaagaagaagagactgcaggcgttccagggataccaggtcaccatgaaaactgccaaagtggcagcctccgactggaccttccttcactgcctgccgaggaagcctgaagaggtggacgacgaggtgttctactccccgcgctccttggtgtttcctgaggccgaaaaccggaagtggactatcatggccgtgatggtgtccctcctcaccgactactcgccgcaactgcagaagcctaagttctag

SEQ ID NO: 154 - DNA, 1065 bp, synthetic construct
  misc_feature 1..1065: note = Synthetic construct
  source 1..1065: mol_type = other DNA
atgttattcaaccttagaattctccttaacaacgccgccttccggaatgggcataactttatggtccgcaatttccgctgtggacagcctctgcaaaacaaggtccagctcaagggccgggatctgctgactctcaagaacttcactggggaagaaatcaagtacatgctctggctgagcgccgacctcaagttccgcatcaagcagaagggagagtacctcccgctgctccaagggaagtccctgggcatgatcttcgagaagagatccacccgcaccagactttccactgagactggcttcgccttgctgggaggccacccatgcttcctgacgacccaggacattcaccttggcgtgaacgagtccctgactgacaccgcaagggtgttgtcctcgatggccgacgccgtgcttgcccgggtgtacaagcagagcgatcttgacaccctggctaaggaagcttccattcccatcatcaacggtctgagcgacctgtaccacccgattcagatcctggcggactacctaaccctgcaagagcactatagctccctgaagggcctcacactttcatggatcggcgacggcaacaacatcctgcactctattatgatgagcgctgccaaattcggcatgcacctccaagccgccacgcctaaaggctacgagcccgacgcctcggtgaccaagcttgcggagcagtacgcgaaggaaaacggcaccaagctgcttctcaccaacgatcctctggaagcggcccatggtggcaacgtgctcattaccgacacttggatctccatgggacaggaggaggaaaagaagaagcggctccaggcgtttcagggttaccaggtcaccatgaaaaccgccaaggtcgcagcctccgactggaccttccttcattgccttccgcgcaagcccgaagaagtggacgatgaagtgttttactcacctcggtcactcgtgttcccggaagcagagaacaggaaatggaccattatggccgtgatggtgtccctgctcaccgattacagtccgcaactgcagaagcccaagttctag

SEQ ID NO: 155 - DNA, 1065 bp, synthetic construct
  misc_feature 1..1065: note = Synthetic construct
  source 1..1065: mol_type = other DNA
atgcttttcaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcaaatcctggccgattacctcaccctgcaagaacactacagctccctgaagggtctgacattgtcctggatcggcgacggcaacaacattctccattccatcatgatgtccgccgcaaaattcggcatgcatcttcaagccgccacgcctaagggttacgaacccgacgcttccgtgactaagctcgccgagcagtacgctaaggagaacggaaccaagctgctgctgactaacgacccgctagaagcagcccacgggggcaacgtgcttattactgacacctggatctccatgggccaggaggaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcacttctcaccgactacagcccgcagcttcagaagcccaagttctag

SEQ ID NO: 156 - DNA, 1065 bp, synthetic construct
  misc_feature 1..1065: note = Synthetic construct
  source 1..1065: mol_type = other DNA
atgcttttcaatctccgcatcctccttaacaacgccgcgtttagaaacggccacaacttcatggtccggaacttcagatgtggccagccgcttcaaaacaaggtccagcttaagggccgggatctcctcacccttaaaaacttcaccggcgaagagatcaagtacatgctctggctctccgcggaccttaagttccgcattaagcagaagggggaataccttccgctgcttcaaggaaagagcctcggcatgatctttgagaagcgctcaaccaggaccaggctttctactgaaactgggttcgcgcttctcggcggtcatccctgcttcctcacgacccaagacatccacctcggagtgaacgaatccctcacggatactgcccgcgtgctttcgagcatggcagacgccgtgctcgcccgggtgtacaaacagtccgatctcgacactctcgccaaggaggcgtcaattcctattatcaacggtcttagtgacctttaccacccgatccagatcctcgccgattacctcacactccaagaacactacagctcccttaagggtcttaccctctcctggatcggcgacggcaacaacattctccactccatcatgatgtccgccgcaaagttcggcatgcatcttcaagccgccaccccgaagggctacgagcctgatgcttccgtgactaagctcgccgagcagtacgctaaggagaacggaaccaagcttcttctcactaacgacccactcgaagcagcccatgggggcaacgtgcttatcactgacacctggatctccatgggccaggaagaagagaagaagaagcggctccaggcgttccagggatatcaggtcaccatgaaaaccgccaaggtcgctgcctccgactggacctttctccactgcctccctcgcaaacctgaagaagtggacgacgaggtgttctactcgccccggagcctcgtgttccccgaggccgagaatagaaagtggaccattatggccgtgatggtgtcactcctcaccgactacagcccgcagcttcagaagcccaagttctag

SEQ ID NO: 157 - DNA, 1065 bp, synthetic construct
  misc_feature 1..1065: note = Synthetic construct
  source 1..1065: mol_type = other DNA
atgcttttcaatctccgcatcctccttaacaacgccgcgtttagaaacggacataacttcatggtccggaacttcagatgtggacagccgcttcaaaacaaggtccagctgaagggtcgggatcttctgaccctgaagaactttaccggagaagagatcaagtacatgctctggctctccgcggacttgaagttccgcattaagcagaagggagaatacctcccgctgcttcaaggaaagagcctcggaatgatttttgagaagcgctcaaccaggacccgcctttctactgaaactggattcgcgctgctgggtggacacccctgcttcctgacgacccaggacatccacctcggagtgaacgaatccctcactgataccgcccgggtgttatcgagcatggcagatgccgtgctggccagggtgtacaaacagtccgatctggacactctggccaaggaggcgtcaattcctatcatcaacggacttagtgacctctaccatccgattcaaatcctggccgactacctcaccctgcaagaacactacagctccctgaagggtctgacattgtcctggatcggagatggaaacaacattctccactccatcatgatgtccgccgcaaaattcggaatgcatcttcaagccgccacgcctaagggttacgaacccgacgcttccgtgactaagctcgccgagcagtacgctaaggagaacggtaccaagcttctcctgaccaacgacccactagaagcagcccacggtggaaacgtgcttattactgacacttggatctccatgggacaggaggaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccgcggagcctcgtgttccccgaggccgagaatagaaagtggaccatcatggccgtgatggtgtcactgctcaccgactacagcccgcagcttcagaagcccaagttctag

SEQ ID NO: 158 - DNA, 1065 bp, synthetic construct
  misc_feature 1..1065: note = Synthetic construct
  source 1..1065: mol_type = other DNA
atgcttttcaacctccgcattctcctcaacaacgctgccttccggaatggacataacttcatggtccggaacttcagatgcggacagccgcttcagaacaaggtccagcttaaggggagagatctccttaccctcaaaaacttcactggcgaagaaatcaagtacatgctctggcttagtgcggatctcaagttccgcatcaagcagaagggagaatacctcccgctccttcaaggaaagagcctcggcatgatttttgagaagaggtccaccagaactcgcctttcaaccgagactgggttcgccctgcttggcggtcacccctgcttcctcactacccaagacatccacctcggcgtgaacgagagccttaccgacaccgcccgcgtgctctcctcaatggccgacgctgtgctcgcccgggtgtacaagcagtccgaccttgatactctcgccaaggaggcctccatcccaattatcaacgggctctctgatctctaccaccctatccaaatcctcgcggactacctcaccctccaagagcactatagctcgctcaagggcctcaccctttcctggattggcgacggcaacaacattcttcactcgatcatgatgtccgccgccaagttcggcatgcatctccaagccgcgacccccaagggctacgagcctgacgcatccgtgaccaagctcgccgagcagtacgcgaaggaaaatggcaccaagcttcttctcaccaacgacccccttgaggccgctcatggcggcaacgtgctcatcactgacacttggatcagcatgggccaggaggaggaaaagaagaagcgccttcaggcattccagggttaccaggtcaccatgaaaaccgccaaagtggccgcctccgactggacctttcttcactgtctcccgcggaagcctgaagaagtggatgacgaagtgttttactcccctcggtcactcgtgttcccggaagcagaaaacaggaagtggaccattatggcggtcatggtgtccctcctcaccgactacagcccgcagcttcagaaacccaagttctag

SEQ ID NO: 159 - DNA, 1065 bp, synthetic construct
  misc_feature 1..1065: note = Synthetic construct
  source 1..1065: mol_type = other DNA
atgcttttcaatctccgcatcctccttaacaacgcagcgtttagaaacggtcacaacttcatggtccggaacttccgctgtggacagccgcttcaaaacaaggtccagctgaagggtcgggaccttctgaccctgaagaactttactggagaagagatcaagtacatgctttggctgtccgcggacttgaagttccgcattaagcagaagggagaataccttccgctgctccaaggaaagagcctgggaatgatctttgagaagcgctcaaccaggacccgcctttctactgaaactggattcgcgctgctgggtggtcacccttgcttcctgacgacccaggacattcacctcggagtgaacgagtccctcactgataccgccagagtgttatcgagcatggcagatgccgtgctggctagggtgtacaaacagtccgatctggacaccctggccaaggaggcatcaattcctattatcaacggacttagtgacctctaccatccgattcaaatcctggccgattacctcaccctgcaagaacactacagctccctgaagggtctgacattgtcctggatcggagatggaaacaacattctccattccatcatgatgtccgcggccaagttcggaatgcatctccaagccgccacgccgaaaggatacgagccggacgcttccgtgactaagctcgccgagcagtacgctaaggagaacggaaccaagcttctgctgactaacgacccgctagaagccgcccacggtggaaacgtgcttattactgacacctggatctccatgggacaggaagaagagaaaaagaagcggctgcaggcgttccagggatatcaggtcaccatgaaaaccgccaaggtcgccgcctccgactggaccttccttcactgcctgcctcggaagcctgaagaagtggacgacgaggtgttctactcgccgcggagcctcgtgttccctgaggccgagaatagaaagtggaccatcatggccgtgatggtgtcactcctcaccgactacagcccgcagcttcagaagcctaagttctag

SEQ ID NO: 160 - DNA, 1065 bp, synthetic construct
  misc_feature 1..1065: note = Synthetic construct
  source 1..1065: mol_type = other DNA
atgcttttcaatctccgcattctcctcaacaacgcagcctttagaaacggccacaacttcatggtccggaacttcagatgtggccagccgcttcagaacaaggtccagctcaagggccgggacctcctcaccctcaaaaactttaccggcgaagagatcaagtacatgctctggctttcggccgaccttaagttccgcatcaagcagaagggggaataccttccgctgcttcaaggaaagtccctcggcatgatctttgaaaagcgctcgaccaggacccgcctttccactgaaaccgggttcgcgcttctcggtggccacccctgcttcctcaccacccaagacattcacctcggagtgaacgaatcccttaccgataccgcaagagtgctttcgtcgatggccgatgccgtgcttgcgcgggtgtacaagcagtcagatctcgacactctcgccaaggaggcgtccattcctattatcaacggcctttccgacctttaccacccgattcagatcctcgccgattacctcaccctgcaagagcactactcgtcactcaagggtcttaccctctcctggatcggcgacggaaacaacatcctccattcgatcatgatgtccgccgccaaattcggcatgcacctccaagccgcgaccccgaagggttacgagcccgacgcttccgtgaccaagctcgccgaacagtacgctaaggaaaacggcaccaagctcctcctcactaacgaccctctcgaagcagcccatgggggcaacgtgctcattactgacacttggatctcgatgggccaggaagaggagaaaaagaagcggcttcaggcgttccagggatatcaggtcaccatgaaaaccgccaaggtcgctgcctcggactggaccttccttcactgccttccgcgcaagcctgaagaggtggacgatgaggtgttctactccccacggtcccttgtgttccccgaggccgagaataggaagtggaccatcatggccgtgatggtgtcgctcctcactgactactccccgcaacttcagaagcctaagttctag

SEQ ID NO: 161 - DNA, 1065 bp, synthetic construct
  misc_feature 1..1065: note = Synthetic construct
  source 1..1065: mol_type = other DNA
atgctgtttaatctgagaatacttctaaacaacgccgccttccggaatggccataactttatggttcggaatttccgctgcggccagccgctgcagaacaaggtccagctgaagggaagagacttgctgaccctcaagaacttcaccggagaagaaatcaagtatatgctgtggctgtccgccgacctgaaattccgcatcaagcagaagggcgaatatctgccgctgttgcaagggaagtccctggggatgatcttcgagaagaggtccaccagaacacggctttcaaccgaaaccgggtttgcactgctgggtggacacccctgttttctgaccactcaagatatccacctgggcgtgaacgagtcccttaccgacactgctagggtgttgtccagcatggccgatgccgtcctggctcgcgtgtacaagcagtccgacctggataccctggcaaaggaagcgtccattcccattatcaacgggctgtccgacctgtaccatccgattcaaatcctggcggactacctgactctgcaagagcattacagcagcttgaaggggcttactctctcgtggatcggcgacgggaacaacatcctgcactccatcatgatgtccgccgccaagttcgggatgcatttgcaagctgcgaccccgaaaggttacgagcccgatgctagcgtaactaagcttgccgaacagtacgccaaagagaatggtacaaaactgcttctgactaacgacccgctggaagcagcccacggcgggaacgtgctgataaccgacacctggatttcaatggggcaggaggaagagaagaagaagcgactgcaggcgttccaaggctatcaggttaccatgaaaaccgccaaagtggcagccagcgattggactttcctgcactgtctgccgcggaagcccgaggaagttgatgacgaagtattctactcaccccggagcctcgtgttccccgaggccgaaaaccggaagtggactattatggccgtgatggtgtcgctgttgaccgactacagcccgcaactgcagaagccgaagttttag

SEQ ID NO: 162 - DNA, 1065 bp, synthetic construct
  misc_feature 1..1065: note = Synthetic construct
  source 1..1065: mol_type = other DNA
atgcttttcaacctgaggatccttttgaacaacgccgcctttcgcaacggccacaactttatggtccgcaatttccgctgcgggcagccgctgcagaacaaggtccagctgaagggccgggatctgctgaccctgaagaacttcaccggggaggaaatcaagtacatgctttggctctccgccgatctgaagttcagaatcaagcagaagggagagtacctcccgttgctgcaaggaaagtcactcggaatgattttcgaaaagagaagcactaggacccgcctctcaactgaaaccgggttcgcgctgctcgggggccatccgtgtttcctgactacccaagacatccacctgggagtgaacgagtcgctgaccgacaccgcacgcgtgctgtcatccatggcggacgcagtgcttgcccgggtgtacaagcagtcggacctggacactcttgccaaggaggcatcaatccccatcattaacggactgtccgatctctaccacccgattcagatcctggctgactacctaaccctgcaagagcactactcaagcctgaaggggctgaccctgtcgtggatcggggacggcaacaacattctgcactccatcatgatgtcggcggctaagttcgggatgcatttgcaagcggcaactccgaagggttatgaacccgacgcctccgtgaccaagctggccgaacagtacgccaaggaaaacggaaccaagttgctgctgactaatgatcccctggaggcggcccacggggggaacgtgctgataaccgatacctggatctccatggggcaggaagaagagaagaaaaagcggctgcaggcattccagggataccaggtcaccatgaaaaccgcaaaagtggcagccagcgactggactttcctccattgcctgccgcgaaagccggaggaggtcgatgacgaggtgttctactccccgcggtcgctggtgttcccggaggcggaaaaccggaagtggaccattatggccgtgatggtgtcactcctgactgactacagcccgcaactgcagaagccgaagttctag

SEQ ID NO: 163 - DNA, 1074 bp, synthetic construct
  misc_feature 1..1074: note = Synthetic construct
  source 1..1074: mol_type = other DNA
atgcttttcaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagataagtgaa

SEQ ID NO: 164 - DNA, 1073 bp, synthetic construct
  misc_feature 1..1073: note = Synthetic construct
  source 1..1073: mol_type = other DNA
atgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcctggccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaataagtga

SEQ ID NO: 165 - DNA, 1073 bp, synthetic construct; identical to SEQ ID NO: 164
  misc_feature 1..1073: note = Synthetic construct
  source 1..1073: mol_type = other DNA

SEQ ID NO: 166 - DNA, 1074 bp, synthetic construct; identical to SEQ ID NO: 163
  misc_feature 1..1074: note = Synthetic construct
  source 1..1074: mol_type = other DNA

SEQ ID NO: 167 - DNA, 1073 bp, synthetic construct
  misc_feature 1..1073: note = Synthetic construct
  source 1..1073: mol_type = other DNA
atgcttttcaacctgagaatcctcttgaacaatgctgcttttcggaatggccacaactttatggttcggaacttccgttgcggccagcctttacaaaacaaggtccagctgaagggccgggatttgctcacactaaagaactttactggagaagagatcaagtacatgctatggctgtcggccgacctgaagttccgtatcaagcagaagggagaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctcgataccttggcaaaggaggcttccattcccatcatcaacggcctgagcgacctgtaccacccaatccaaatcctggctgactacctgaccctgcaagagcactacagcagcctgaagggtctgaccctgtcatggattggcgatggaaacaatattctgcactccatcatgatgtccgccgcgaagttcggaatgcatctgcaagccgccactccaaaaggatacgaaccggatgcatccgtgaccaagttggcggaacagtacgcgaaggagaacggaaccaagctcctgctgactaacgacccgctcgaggctgcgcatgggggtaacgtgctgattacggacacctggatctccatggggcaggaggaagagaagaagaagagactgcaggcattccaggggtaccaggtcaccatgaaaaccgcaaaagtggcagcttcggactggactttcctgcattgcctgccgaggaagccggaggaagtcgacgacgaagtgttctactcgcctcggtccctggtgttccccgaggccgaaaaccggaagtggaccatcatggccgtgatggtgtccttgctgactgactatagcccgcagctgcagaagcctaagttctagataagtga

SEQ ID NO: 168 - DNA, 1073 bp, synthetic construct; identical to SEQ ID NO: 164
  misc_feature 1..1073: note = Synthetic construct
  source 1..1073: mol_type = other DNA

SEQ ID NO: 169 - DNA, 1074 bp, synthetic construct; identical to SEQ ID NO: 163
  misc_feature 1..1074: note = Synthetic construct
  source 1..1074: mol_type = other DNA

SEQ ID NO: 170 - DNA, 1073 bp, synthetic construct; identical to SEQ ID NO: 164
  misc_feature 1..1073: note = Synthetic construct
  source 1..1073: mol_type = other DNA

SEQ ID NO: 171 - DNA, 1073 bp, synthetic construct; identical to SEQ ID NO: 167
  misc_feature 1..1073: note = Synthetic construct
  source 1..1073: mol_type = other DNA

SEQ ID NO: 172 - DNA, 1074 bp, synthetic construct; identical to SEQ ID NO: 163
  misc_feature 1..1074: note = Synthetic construct
  source 1..1074: mol_type = other DNA

SEQ ID NO: 173 - DNA, 1073 bp, synthetic construct; identical to SEQ ID NO: 164
  misc_feature 1..1073: note = Synthetic construct
  source 1..1073: mol_type = other DNA

SEQ ID NO: 174 - DNA, 1073 bp, synthetic construct; identical to SEQ ID NO: 167
  misc_feature 1..1073: note = Synthetic construct
  source 1..1073: mol_type = other DNA

SEQ ID NO: 175 - DNA, 1065 bp, synthetic construct
  misc_feature 1..1065: note = Synthetic construct
  source 1..1065: mol_type = other DNA
atgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctga

SEQ ID NO: 176 - DNA, 1065 bp, synthetic construct; identical to SEQ ID NO: 175
  misc_feature 1..1065: note = Synthetic construct
  source 1..1065: mol_type = other DNA

SEQ ID NO: 177 - DNA, 1065 bp, synthetic construct; identical to SEQ ID NO: 175
  misc_feature 1..1065: note = Synthetic construct
  source 1..1065: mol_type = other DNA

SEQ ID NO: 178 - DNA, 1074 bp, synthetic construct
  misc_feature 1..1074: note = Synthetic construct
  source 1..1074: mol_type = other DNA
atgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaataagtgaa

SEQ ID NO: 179 - DNA, 1077 bp, synthetic construct
  misc_feature 1..1077: note = Synthetic construct
  source 1..1077: mol_type = other DNA
atgggcgtcttcaacctgcggatcctgctgaacaacgccgccttccggaacggccacaacttcatggtccgcaacttcagatgcggccagcccctgcagaacaaggtgcagctgaagggccgggacctgctgaccctgaagaacttcaccggcgaagagatcaagtacatgctgtggctgagcgccgacctgaagttccggatcaagcagaagggcgagtacctgcccctgctgcaaggcaagagcctgggcatgatcttcgagaagcggagcacccggacccggctgagcaccgagacaggctttgccctgctgggaggccacccctgctttctgaccacccaggacatccacctgggcgtgaacgagagcctgaccgacaccgccagagtgctgagcagcatggccgacgccgtgctggcccgggtgtacaagcagagcgacctggacaccctggccaaagaggccagcatccccatcatcaacggcctgagcgacctgtaccaccccatccagatcctggccgactacctgaccctgcaggaacactacagctccctgaagggcctgaccctgagctggatcggcgacggcaacaacatcctgcacagcatcatgatgagcgccgccaagttcggcatgcatctgcaggccgccacccccaagggctacgagcctgatgccagcgtgaccaagctggccgagcagtacgccaaagagaacggcaccaagctgctgctgaccaacgaccccctggaagccgcccacggcggcaacgtgctgatcaccgacacctggatcagcatgggccaggaagaggaaaagaagaagcggctgcaggccttccagggctaccaggtcacaatgaagaccgccaaggtggccgccagcgactggaccttcctgcactgcctgccccggaagcccgaagaggtggacgacgaggtgttctacagcccccggtccctggtgttccccgaggccgagaaccggaagtggaccattatggccgtgatggtgtccctgctgaccgactactccccccagctgcagaagcccaagttctagataagtgaa

SEQ ID NO: 180 - DNA, 1077 bp, synthetic construct
  misc_feature 1..1077: note = Synthetic construct
  source 1..1077: mol_type = other DNA
atgggcgtcttcaacctgcggatcctgctgaacaacgccgccttccggaacggccacaacttcatggtccgcaacttcagatgcggccagcccctgcagaacagggtgcagctgaagggccgggacctgctgaccctgaagaacttcaccggcgaagagatcaggtacatgctgtggctgagcgccgacctgaagttccggatcaagcagaagggcgagtacctgcccctgctgcaaggcaagagcctgggcatgatcttcgagaagcggagcacccggacccggctgagcaccgagacaggctttgccctgctgggaggccacccctgctttctgaccacccaggacatccacctgggcgtgaacgagagcctgaccgacaccgccagagtgctgagcagcatggccgacgccgtgctggcccgggtgtacaagcagagcgacctggacaccctggccaaagaggccagcatccccatcatcaacggcctgagcgacctgtaccaccccatccagatcctggccgactacctgaccctgcaggaacactacagctccctgaagggcctgaccctgagctggatcggcgacggcaacaacatcctgcacagcatcatgatgagcgccgccaagttcggcatgcatctgcaggccgccacccccaagggctacgagcctgatgccagcgtgaccaagctggccgagcagtacgccaaagagaacggcaccaagctgctgctgaccaacgaccccctggaagccgcccacggcggcaacgtgctgatcaccgacacctggatcagcatgggccaggaagaggaaaagaagaagcggctgcaggccttccagggctaccaggtcacaatgaagaccgccaaggtggccgccagcgactggaccttcctgcactgcctgccccggaagcccgaagaggtggacgacgaggtgttctacagcccccggtccctggtgttccccgaggccgagaaccggaagtggaccattatggccgtgatggtgtccctgctgaccgactactccccccagctgcagaagcccaagttctagataagtgaa

SEQ ID NO: 181 - DNA, 1077 bp, synthetic construct
  misc_feature 1..1077: note = Synthetic construct
  source 1..1077: mol_type = other DNA
atgctggtcttcaacctgcggatcctgctgaacaacgccgccttccggaacggccacaacttcatggtccgcaacttcagatgcggccagcccctgcagaacagggtgcagctgaagggccgggacctgctgaccctgaagaacttcaccggcgaagagatcaggtacatgctgtggctgagcgccgacctgaagttccggatcaagcagaagggcgagtacctgcccctgctgcaaggcaagagcctgggcatgatcttcgagaagcggagcacccggacccggctgagcaccgagacaggctttgccctgctgggaggccacccctgctttctgaccacccaggacatccacctgggcgtgaacgagagcctgaccgacaccgccagagtgctgagcagcatggccgacgccgtgctggcccgggtgtacaagcagagcgacctggacaccctggccaaagaggccagcatccccatcatcaacggcctgagcgacctgtaccaccccatccagatcctggccgactacctgaccctgcaggaacactacagctccctgaagggcctgaccctgagctggatcggcgacggcaacaacatcctgcacagcatcatgatgagcgccgccaagttcggcatgcatctgcaggccgccacccccaagggctacgagcctgatgccagcgtgaccaagctggccgagcagtacgccaaagagaacggcaccaagctgctgctgaccaacgaccccctggaagccgcccacggcggcaacgtgctgatcaccgacacctggatcagcatgggccaggaagaggaaaagaagaagcggctgcaggccttccagggctaccaggtcacaatgaagaccgccaaggtggccgccagcgactggaccttcctgcactgcctgccccggaagcccgaagaggtggacgacgaggtgttctacagcccccggtccctggtgttccccgaggccgagaaccggaagtggaccattatggccgtgatggtgtccctgctgaccgactactccccccagctgcagaagcccaagttctagataagtgaa

SEQ ID NO: 182 - DNA, 1074 bp, synthetic construct
  misc_feature 1..1074: note = Synthetic construct
  source 1..1074: mol_type = other DNA
atgctgttcaacctgaggatcctgctgaacaacgcagctttcaggaacggccacaacttcatggtgaggaacttccggtgcggccagcccctgcagaacaaggtgcagctgaagggcagggacctgctgaccctgaagaacttcaccggagaggagatcaagtacatgctgtggctgagcgcagacctgaagttcaggatcaagcagaagggagagtacctgcccctgctgcaggggaagtccctgggcatgatcttcgagaagaggagtaccaggaccaggctgagcaccgaaaccggcttcgccctgctgggaggacacccctgcttcctgaccacccaggacatccacctgggcgtgaacgagagtctgaccgacaccgccagggtgctgtctagcatggccgacgccgtgctggccagggtgtacaagcagtcagacctggacaccctggctaaggaggccagcatccccatcatcaacggcctgagcgacctgtaccaccccatccagatcctggctgactacctgaccctgcaggagcactacagctctctgaagggcctgaccctgagctggatcggcgacgggaacaacatcctgcacagcatcatgatgagcgccgccaagttcggcatgcacctgcaggccgctacccccaagggttacgagcccgacgccagcgtgaccaagctggcagagcagtacgccaaggagaacggcaccaagctgctgctgaccaacgaccccctggaggccgcccacggaggcaacgtgctgatcaccgacacctggatcagcatgggacaggaggaggagaagaagaagcggctgcaggctttccagggttaccaggtgaccatgaagaccgccaaggtggctgccagcgactggaccttcctgcactgcctgcccaggaagcccgaggaggtggacgacgaggtgttctactctcccaggagcctggtgttccccgaggccgagaacaggaagtggaccatcatggctgtgatggtgtccctgctgaccgactacagcccccagctgcagaagcccaagttctgaataagtgaa

SEQ ID NO: 183 - DNA, 1074 bp, synthetic construct
  misc_feature 1..1074: note = Synthetic construct
  source 1..1074: mol_type = other DNA
atgctgttcaacctgaggatcctgctgaacaacgcagctttcaggaacggccacaacttcatggtgaggaacttccggtgcggccagcccctgcagaacaaggtgcagctgaagggcagggacctgctgaccctgaagaacttcaccggagaggagatcaagtacatgctgtggctgagcgcagacctgaagttcaggatcaagcagaagggagagtacctgcccctgctgcaggggaagtccctgggcatgatcttcgagaagaggagtaccaggaccaggctgagcaccgaaaccggcttcgccctgctgggaggacacccctgcttcctgacgacccaggacatccacctcggagtgaacgaatccctcaccgataccgcccgggtgttatcaagcatggcagatgccgtgctggccagggtgtacaaacagtccgatctggacactctggctaaggaggccagcatccccatcatcaacggcctgagcgacctgtaccaccccatccagatcctggctgactacctgaccctgcaggagcactacagctctctgaagggcctgaccctgagctggatcggcgacgggaacaacatcctgcactccatcatgatgtccgccgcgaagttcggaatgcatctgcaagccgccacgccaaaaggatacgaaccggatgcgcccgtgacaaagttggcggaacagtacgctaaggagaacggaaccaagctgctgctgaccaacgaccccctggaggccgcccacggaggcaacgtgctgatcaccgacacctggatcagcatgggacaggaggaggagaagaagaagcggctgcaggctttccagggttaccaggtgaccatgaagaccgccaaggtggctgccagcgactggaccttcctgcactgcctgcccaggaagcccgaggaggtggacgacgaggtgttctactctcccaggagcctggtgttccccgaggccgagaacaggaagtggaccatcatggctgtgatggtgtccctgctgaccgactacagcccccagctgcagaagcccaagttctgaataagtgaa

SEQ ID NO: 184 - DNA, 1074 bp, synthetic construct; identical to SEQ ID NO: 178
  misc_feature 1..1074: note = Synthetic construct
  source 1..1074: mol_type = other DNA

SEQ ID NO: 185 - DNA, 1074 bp, synthetic construct; identical to SEQ ID NO: 178
  misc_feature 1..1074: note = Synthetic construct
  source 1..1074: mol_type = other DNA

SEQ ID NO: 186 - DNA, 1065 bp, synthetic construct
  misc_feature 1..1065: note = Synthetic construct
  source 1..1065: mol_type = other DNA
atgcttttcaacttgagaatcctgctgaacaacgccgcctttcgcaacggtcacaattttatggtcagaaacttcagatgcggacagcccctccaaaacaaggtccagctgaagggccgcgatctcctcaccctgaagaacttcacgggggaggagatcaagtacatgctgtggctctccgctgacctgaagttcaggatcaagcagaagggagaatatctgccgctgctgcaagggaagtccctggggatgattttcgagaagcggagcacccggactcggctctccactgaaactggtttcgcccttctgggcggtcacccctgcttcctgaccactcaagacattcacctcggagtgaacgagtccttgactgacaccgcccgggtgctgtcgagcatggcagacgccgtgctagcccgcgtgtacaagcagtcagacctcgataccctggccaaggaggcttcgatcccgatcatcaacgggttgtccgacctgtaccacccgattcagattctcgccgactacctcaccctgcaagagcattacagctccctgaaggggcttaccctgtcctggattggcgacggaaacaacatcctgcactccattatgatgtcggcggccaagttcggcatgcacctccaagccgcgacccctaagggttacgaaccagacgcgtcagtgactaagctggccgaacagtacgcaaaggaaaatggcacgaagctgctcctgaccaacgatccgttggaagccgcccatggcggaaatgtgctcatcaccgacacctggatctcgatgggacaggaggaagagaagaagaagcggctgcaggcgttccagggctaccaggtcaccatgaaaactgccaaggtggccgccagcgactggaccttcctgcactgccttccgcgcaagcctgaggaggtggacgatgaagtgttctactctccacggtccctggtgttccccgaggcggagaaccgcaaatggaccatcatggctgtgatggtcagcctgctgaccgattacagccctcagttgcaaaagccgaagttttga

SEQ ID NO: 187 - DNA, 1065 bp, synthetic construct
  misc_feature 1..1065: note = Synthetic construct
  source 1..1065: mol_type = other DNA
atgctgttcaacctccgcatcctcctcaacaacgccgcattcagaaacgggcacaacttcatggtcagaaacttccgctgcgggcaacccctacaaaacaaggtccagctcaaggggcgggacctcctgaccctgaagaacttcaccggcgaagagatcaagtacatgctgtggctctccgccgacctgaagttccgcatcaagcagaagggagagtacctcccgctgctgcaagggaagtcgctggggatgatcttcgagaagcggtcaaccagaacccggctgtcaaccgaaaccgggttcgcactgctggggggacacccgtgcttcctgaccacccaagacatccacctgggagtgaacgaatcgctgaccgacaccgcccgcgtgctgagctcaatggcggacgccgtgctggcccgcgtgtacaagcagtccgacctggacaccctggccaaggaagcgtccatcccgatcatcaacggactgtccgacctgtaccacccgatccagatcctggcagactacctgaccctgcaagaacactacagctccctgaagggcctgaccctgtcatggatcggggacgggaacaacatcctgcactccataatgatgtcagccgccaagttcggaatgcacctccaagccgcaaccccgaagggctacgaaccggacgcatcagtgaccaaactggccgagcagtacgccaaggaaaacggcaccaagctcctgctgaccaacgacccgctggaggccgcacacggggggaacgtgctgatcaccgacacctggatctccatgggacaggaggaggaaaagaagaagcggctgcaggcgttccaggggtaccaggtcaccatgaaaaccgcgaaggtcgcggcatcagactggaccttcctgcactgcctgccccggaagccggaagaggtggacgacgaggtgttctactcgccgcgctcgctggtgttccccgaggcggagaacaggaagtggaccatcatggcggtgatggtcagcctcctgaccgactactcgccgcagctgcagaagccgaagttctga

SEQ ID NO: 188 - DNA, 1074 bp, synthetic construct
  misc_feature 1..1074: note = Synthetic construct
  source 1..1074: mol_type = other DNA
atgcttttcaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctgaataagtaga

SEQ ID NO: 189 - DNA, 1074 bp, synthetic construct
  misc_feature 1..1074: note = Synthetic construct
  source 1..1074: mol_type = other DNA
atgcttttcaacctgagaatcctcttgaacaatgctgcttttcggaatggccacaactttatggttcggaacttccgttgcggccagcctttacaaaacaaggtccagctgaagggccgggatttgctcacactaaagaactttactggagaagagatcaagtacatgctatggctgtcggccgacctgaagttccgtatcaagcagaagggagaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctcgataccttggcaaaggaggcttccattcccatcatcaacggcctgagcgacctgtaccacccaatccaaatcctggctgactacctgaccctgcaagagcactacagcagcctgaagggtctgaccctgtcatggattggcgatggaaacaatattctgcactccatcatgatgtccgccgcgaagttcggaatgcatctgcaagccgccactccaaaaggatacgaaccggatgcatccgtgaccaagttggcggaacagtacgcgaaggagaacggaaccaagctcctgctgactaacgacccgctcgaggctgcgcatgggggtaacgtgctgattacggacacctggatctccatggggcaggaggaagagaagaagaagagactgcaggcattccaggggtaccaggtcaccatgaaaaccgcaaaagtggcagcttcggactggactttcctgcattgcctgccgaggaagccggaggaagtcgacgacgaagtgttctactcgcctcggtccctggtgttccccgaggccgaaaaccggaagtggaccatcatggccgtgatggtgtccttgctgactgactatagcccgcagctgcagaagcctaagttctgaataagtaga

SEQ ID NO: 190 - DNA, 1074 bp, synthetic construct
  misc_feature 1..1074: note = Synthetic construct
  source 1..1074: mol_type = other DNA
atgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcctggccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaataagtaga

SEQ ID NO: 191 - DNA, 1065 bp, synthetic construct; identical to SEQ ID NO: 136
  misc_feature 1..1065: note = Synthetic construct
  source 1..1065: mol_type = other DNA

SEQ ID NO: 192 - DNA, 1065 bp, synthetic construct; identical to SEQ ID NO: 136
  misc_feature 1..1065: note = Synthetic construct
  source 1..1065: mol_type = other DNA

SEQ ID NO: 193 - DNA, 1065 bp, synthetic construct; identical to SEQ ID NO: 136
  misc_feature 1..1065: note = Synthetic construct
  source 1..1065: mol_type = other DNA

SEQ ID NO: 194 - DNA, 1065 bp, synthetic construct; identical to SEQ ID NO: 136
  misc_feature 1..1065: note = Synthetic construct
  source 1..1065: mol_type = other DNA

SEQ ID NO: 195 - DNA, 1065 bp, synthetic construct; identical to SEQ ID NO: 136
  misc_feature 1..1065: note = Synthetic construct
  source 1..1065: mol_type = other DNA

SEQ ID NO: 196 - DNA, 1065 bp, synthetic construct
  misc_feature 1..1065: note = Synthetic construct
  source 1..1065: mol_type = other DNA
atggtgttcaacctccgcatcctcctcaacaacgccgcattcagaaacgggcacaacttcatggtcagaaacttccgctgcgggcaacccctacaaaacaaggtccagctcaaggggcgggacctcctgaccctgaagaacttcaccggcgaagagatcaagtacatgctgtggctctccgccgacctgaagttccgcatcaagcagaagggagagtacctcccgctgctgcaagggaagtcgctggggatgatcttcgagaagcggtcaaccagaacccggctgtcaaccgaaaccgggttcgcactgctggggggacacccgtgcttcctgaccacccaagacatccacctgggagtgaacgaatcgctgaccgacaccgcccgcgtgctgagctcaatggcggacgccgtgctggcccgcgtgtacaagcagtccgacctggacaccctggccaaggaagcgtccatcccgatcatcaacggactgtccgacctgtaccacccgatccagatcctggcagactacctgaccctgcaagaacactacagctccctgaagggcctgaccctgtcatggatcggggacgggaacaacatcctgcactccataatgatgtcagccgccaagttcggaatgcacctccaagccgcaaccccgaagggctacgaaccggacgcatcagtgaccaaactggccgagcagtacgccaaggaaaacggcaccaagctcctgctgaccaacgacccgctggaggccgcacacggggggaacgtgctgatcaccgacacctggatctccatgggacaggaggaggaaaagaagaagcggctgcaggcgttccaggggtaccaggtcaccatgaaaaccgcgaaggtcgcggcatcagactggaccttcctgcactgcctgccccggaagccggaagaggtggacgacgaggtgttctactcgccgcgctcgctggtgttccccgaggcggagaacaggaagtggaccatcatggcggtgatggtcagcctcctgaccgactactcgccgcagctgcagaagccgaagttctga

SEQ ID NO: 197 - DNA, 1065 bp, synthetic construct
  misc_feature 1..1065: note = Synthetic construct
  source 1..1065: mol_type = other DNA
atggtgttcaacctccgcatcctcctcaacaacgccgcattcagaaacgggcacaacttcatggtcagaaacttccgctgcgggcaacccctacaaaaccgggtccagctcaaggggcgggacctcctgaccctgaagaacttcaccggcgaagagatcaagtacatgctgtggctctccgccgacctgaagttccgcatcaagcagaagggagagtacctcccgctgctgcaagggaagtcgctggggatgatcttcgagaagcggtcaaccagaacccggctgtcaaccgaaaccgggttcgcactgctggggggacacccgtgcttcctgaccacccaagacatccacctgggagtgaacgaatcgctgaccgacaccgcccgcgtgctgagctcaatggcggacgccgtgctggcccgcgtgtacaagcagtccgacctggacaccctggccaaggaagcgtccatcccgatcatcaacggactgtccgacctgtaccacccgatccagatcctggcagactacctgaccctgcaagaacactacagctccctgaagggcctgaccctgtcatggatcggggacgggaacaacatcctgcactccataatgatgtcagccgccaagttcggaatgcacctccaagccgcaaccccgaagggctacgaaccggacgcatcagtgaccaaactggccgagcagtacgccaaggaaaacggcaccaagctcctgctgaccaacgacccgctggaggccgcacacggggggaacgtgctgatcaccgacacctggatctccatgggacaggaggaggaaaagaagaagcggctgcaggcgttccaggggtaccaggtcaccatgaaaaccgcgaaggtcgcggcatcagactggaccttcctgcactgcctgccccggaagccggaagaggtggacgacgaggtgttctactcgccgcgctcgctggtgttccccgaggcggagaacaggaagtggaccatcatggcggtgatggtcagcctcctgaccgactactcgccgcagctgcagaagccgaagttctga

SEQ ID NO: 198 - DNA, 1065 bp, synthetic construct
  misc_feature 1..1065: note = Synthetic construct
  source 1..1065: mol_type = other DNA
atggtgttcaacctccgcatcctcctcaacaacgccgcattcagaaacgggcacaacttcatggtcagaaacttccgctgcgggcaacccctacaaaaccgggtccagctcaaggggcgggacctcctgaccctgaagaacttcaccggcgaagagatccggtacatgctgtggctctccgccgacctgaagttccgcatcaagcagaagggagagtacctcccgctgctgcaagggaagtcgctggggatgatcttcgagaagcggtcaaccagaacccggctgtcaaccgaaaccgggttcgcactgctggggggacacccgtgcttcctgaccacccaagacatccacctgggagtgaacgaatcgctgaccgacaccgcccgcgtgctgagctcaatggcggacgccgtgctggcccgcgtgtacaagcagtccgacctggacaccctggccaaggaagcgtccatcccgatcatcaacggactgtccgacctgtaccacccgatccagatcctggcagactacctgaccctgcaagaacactacagctccctgaagggcctgaccctgtcatggatcggggacgggaacaacatcctgcactccataatgatgtcagccgccaagttcggaatgcacctccaagccgcaaccccgaagggctacgaaccggacgcatcagtgaccaaactggccgagcagtacgccaaggaaaacggcaccaagctcctgctgaccaacgacccgctggaggccgcacacggggggaacgtgctgatcaccgacacctggatctccatgggacaggaggaggaaaagaagaagcggctgcaggcgttccaggggtaccaggtcaccatgaaaaccgcgaaggtcgcggcatcagactggaccttcctgcactgcctgccccggaagccggaagaggtggacgacgaggtgttctactcgccgcgctcgctggtgttccccgaggcggagaacaggaagtggaccatcatggcggtgatggtcagcctcctgaccgactactcgccgcagctgcagaagccgaagttctga

SEQ ID NO: 199 - DNA, 1065 bp, synthetic construct
  misc_feature 1..1065: note = Synthetic construct
  source 1..1065: mol_type = other DNA
atgctggtcaacctccgcatcctcctcaacaacgccgcattcagaaacgggcacaacttcatggtcagaaacttccgctgcgggcaacccctacaaaacaaggtccagctcaaggggcgggacctcctgaccctgaagaacttcaccggcgaagagatcaagtacatgctgtggctctccgccgacctgaagttccgcatcaagcagaagggagagtacctcccgctgctgcaagggaagtcgctggggatgatcttcgagaagcggtcaaccagaacccggctgtcaaccgaaaccgggttcgcactgctggggggacacccgtgcttcctgaccacccaagacatccacctgggagtgaacgaatcgctgaccgacaccgcccgcgtgctgagctcaatggcggacgccgtgctggcccgcgtgtacaagcagtccgacctggacaccctggccaaggaagcgtccatcccgatcatcaacggactgtccgacctgtaccacccgatccagatcctggcagactacctgaccctgcaagaacactacagctccctgaagggcctgaccctgtcatggatcggggacgggaacaacatcctgcactccataatgatgtcagccgccaagttcggaatgcacctccaagccgcaaccccgaagggctacgaaccggacgcatcagtgaccaaactggccgagcagtacgccaaggaaaacggcaccaagctcctgctgaccaacgacccgctggaggccgcacacggggggaacgtgctgatcaccgacacctggatctccatgggacaggaggaggaaaagaagaagcggctgcaggcgttccaggggtaccaggtcaccatgaaaaccgcgaaggtcgcggcatcagactggaccttcctgcactgcctgccccggaagccggaagaggtggacgacgaggtgttctactcgccgcgctcgctggtgttccccgaggcggagaacaggaagtggaccatcatggcggtgatggtcagcctcctgaccgactactcgccgcagctgcagaagccgaagttctga

SEQ ID NO: 200 - DNA, 1065 bp, synthetic construct
  misc_feature 1..1065: note = Synthetic construct
  source 1..1065: mol_type = other DNA
atgctggtcaacctccgcatcctcctcaacaacgccgcattcagaaacgggcacaacttcatggtcagaaacttccgctgcgggcaacccctacaaaaccgggtccagctcaaggggcgggacctcctgaccctgaagaacttcaccggcgaagagatcaagtacatgctgtggctctccgccgacctgaagttccgcatcaagcagaagggagagtacctcccgctgctgcaagggaagtcgctggggatgatcttcgagaagcggtcaaccagaacccggctgtcaaccgaaaccgggttcgcactgctggggggacacccgtgcttcctgaccacccaagacatccacctgggagtgaacgaatcgctgaccgacaccgcccgcgtgctgagctcaatggcggacgccgtgctggcccgcgtgtacaagcagtccgacctggacaccctggccaaggaagcgtccatcccgatcatcaacggactgtccgacctgtaccacccgatccagatcctggcagactacctgaccctgcaagaacactacagctccctgaagggcctgaccctgtcatggatcggggacgggaacaacatcctgcactccataatgatgtcagccgccaagttcggaatgcacctccaagccgcaaccccgaagggctacgaaccggacgcatcagtgaccaaactggccgagcagtacgccaaggaaaacggcaccaagctcctgctgaccaacgacccgctggaggccgcacacggggggaacgtgctgatcaccgacacctggatctccatgggacaggaggaggaaaagaagaagcggctgcaggcgttccaggggtaccaggtcaccatgaaaaccgcgaaggtcgcggcatcagactggaccttcctgcactgcctgccccggaagccggaagaggtggacgacgaggtgttctactcgccgcgctcgctggtgttccccgaggcggagaacaggaagtggaccatcatggcggtgatggtcagcctcctgaccgactactcgccgcagctgcagaagccgaagttctga

SEQ ID NO: 201 - DNA, 1065 bp, synthetic construct
  misc_feature 1..1065: note = Synthetic construct
  source 1..1065: mol_type = other DNA
atgctggtcaacctccgcatcctcctcaacaacgccgcattcagaaacgggcacaacttcatggtcagaaacttccgctgcgggcaacccctacaaaaccgggtccagctcaaggggcgggacctcctgaccctgaagaacttcaccggcgaagagatccggtacatgctgtggctctccgccgacctgaagttccgcatcaagcagaagggagagtacctcccgctgctgcaagggaagtcgctggggatgatcttcgagaagcggtcaaccagaacccggctgtcaaccgaaaccgggttcgcactgctggggggacacccgtgcttcctgaccacccaagacatccacctgggagtgaacgaatcgctgaccgacaccgcccgcgtgctgagctcaatggcggacgccgtgctggcccgcgtgtacaagcagtccgacctggacaccctggccaaggaagcgtccatcccgatcatcaacggactgtccgacctgtaccacccgatccagatcctggcagactacctgaccctgcaagaacactacagctccctgaagggcctgaccctgtcatggatcggggacgggaacaacatcctgcactccataatgatgtcagccgccaagttcggaatgcacctccaagccgcaaccccgaagggctacgaaccggacgcatcagtgaccaaactggccgagcagtacgccaaggaaaacggcaccaagctcctgctgaccaacgacccgctggaggccgcacacggggggaacgtgctgatcaccgacacctggatctccatgggacaggaggaggaaaagaagaagcggctgcaggcgttccaggggtaccaggtcaccatgaaaaccgcgaaggtcgcggcatcagactggaccttcctgcactgcctgccccggaagccggaagaggtggacgacgaggtgttctactcgccgcgctcgctggtgttccccgaggcggagaacaggaagtggaccatcatggcggtgatggtcagcctcctgaccgactactcgccgcagctgcagaagccgaagttctga

SEQ ID NO: 202 - DNA, 1065 bp, synthetic construct
  misc_feature 1..1065: note = Synthetic construct
  source 1..1065: mol_type = other DNA
atgctggtcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctga

SEQ ID NO: 203 - DNA, 1065 bp, synthetic construct
  misc_feature 1..1065: note = Synthetic construct
  source 1..1065: mol_type = other DNA
atgctggtcaacctgcgcatcctgctgaacaacgccgccttccgcaacggccacaacttcatggtgcgcaacttccgctgcggccagcccctgcagaaccgggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctga

SEQ ID NO: 204 - DNA, 1065 bp, synthetic construct
  misc_feature 1..1065: note = Synthetic construct
  source 1..1065: mol_type = other DNA
atgcttgtcaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctag

SEQ ID NO: 205 - DNA, 1065 bp, synthetic construct
  misc_feature 1..1065: note = Synthetic construct
  source 1..1065: mol_type = other DNA
atgcttgtcaatctccgcatcctccttaacaacgccgcgtttagaaacggccacaacttcatggtccggaacttcagatgtggccagccgcttcaaaaccgggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctag

SEQ ID NO: 206 - DNA, 1068 bp, synthetic construct
  misc_feature 1..1068: note = Synthetic construct
  source 1..1068: mol_type = other DNA
atgggccttgtcaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctag

SEQ ID NO: 207 - DNA, 1068 bp, synthetic construct
  misc_feature 1..1068: note = Synthetic construct
  source 1..1068: mol_type = other DNA
atgggccttgtcaatctccgcatcctccttaacaacgccgcgtttagaaacggccacaacttcatggtccggaacttcagatgtggccagccgcttcaaaaccgggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctag

SEQ ID NO: 208 - DNA, 1071 bp, synthetic construct
  misc_feature 1..1071: note = Synthetic construct
  source 1..1071: mol_type = other DNA
atgggcggacttgtcaatctccgcatcctccttaacaacgccgcgtttagaaacggccacaacttcatggtccggaacttcagatgtggccagccgcttcaaaacaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctag

SEQ ID NO: 209 - DNA, 1077 bp, synthetic construct
  misc_feature 1..1077: note = Synthetic construct
  source 1..1077: mol_type = other DNA
atggcccttttcaatctccgcatcctccttaacaacgccgcgtttagaaacggccacaacttcatggtccggaacttcagatgtggccagccgcttcaaggcaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagataagtgaa

SEQ ID NO: 210 - DNA, 1077 bp, synthetic construct
  misc_feature 1..1077: note = Synthetic construct
  source 1..1077: mol_type = other DNA
atggcccttttcaatctccgcatcctccttaacaacgccgcgtttagaaacggccacaacttcatggtccggaacttcagatgtggccagccgcttcaaggccgggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagataagtgaa

SEQ ID NO: 211 - DNA, 1077 bp, synthetic construct
  misc_feature 1..1077: note = Synthetic construct
  source 1..1077: mol_type = other DNA
atggcccttttcaatctccgcatcctccttaacaacgccgcgtttagaaacggccacaacttcatggtccggaacttcagatgtggccagccgcttcaaggccgggtccagctgaagggccgggatcttctgaccctgaagaactttactggcgaagagatcaggtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagataagtgaa

SEQ ID NO: 212 - DNA, 1077 bp, synthetic construct
  misc_feature 1..1077: note = Synthetic construct
  source 1..1077: mol_type = other DNA
atggcccttgtcaatctccgcatcctccttaacaacgccgcgtttagaaacggccacaacttcatggtccggaacttcagatgtggccagccgcttcaaggcagggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctaagtgaataga

SEQ ID NO: 213 - DNA, 1077 bp, synthetic construct
  misc_feature 1..1077: note = Synthetic construct
  source 1..1077: mol_type = other DNA
atggcccttttcaatctccgcatcctccttaacaacgccgcgtttagaaacggccacaacttcatggtccggaacttcagatgtggccagccgcttcaagtcaaggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagataagtgaa

SEQ ID NO: 214 - DNA, 1077 bp, synthetic construct
  misc_feature 1..1077: note = Synthetic construct
  source 1..1077: mol_type = other DNA
atggcccttttcaatctccgcatcctccttaacaacgccgcgtttagaaacggccacaacttcatggtccggaacttcagatgtggccagccgcttcaagtcagggtccagctgaagggccgggatcttctgaccctgaagaactttactggcgaagagatcaagtacatgctctggctctccgcggacttgaagttccgcattaagcagaagggggaataccttccgctgcttcaaggaaagagcctcggcatgatctttgagaagcgctcaaccaggacccgcctttctactgaaactgggttcgcgctgctcggtggccacccctgcttcctgacgacccaggacatccacctcggagtgaacgaatccctcaccgataccgcccgggtgttatcgagcatggcagatgccgtgctggccagggtgtacaaacagtccgatctggacactctggccaaggaggcgtcaattcctattatcaacggccttagtgacctctaccatccgattcagatcctggccgattacctcaccctgcaagaacactacagctccctgaagggtctgacattgtcctggatcggcgacggcaacaacattctccattccatcatgatgtccgccgcaaaattcggcatgcatcttcaagccgccacgccgaagggttacgagcccgacgcttccgtgactaagctcgccgagcagtacgctaaggagaacggaaccaagcttctgctgactaacgacccactagaagcagcccacgggggcaacgtgcttattactgacacctggatctccatgggccaggaagaagagaaaaagaagcggctgcaggcgttccagggatatcaggtcaccatgaaaaccgccaaggtcgctgcctccgactggaccttcctgcactgcctgcctcgcaagcctgaagaagtggacgacgaggtgttctactcgccacggagcctcgtgttccccgaggccgagaatagaaagtggaccatcatggccgtgatggtgtcactgctcaccgactacagcccgcagcttcagaagcccaagttctagataagtgaa

SEQ ID NO: 215 - DNA, 1065 bp, synthetic construct; identical to SEQ ID NO: 175
  misc_feature 1..1065: note = Synthetic construct
  source 1..1065: mol_type = other DNA

SEQ ID NO: 216 - DNA, 1068 bp, synthetic construct
  misc_feature 1..1068: note = Synthetic construct
  source 1..1068: mol_type = other DNA
atgggagtattcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctga

SEQ ID NO: 217 - DNA, 1068 bp, synthetic construct
  misc_feature 1..1068: note = Synthetic construct
  source 1..1068: mol_type = other DNA
atgggagtattcaacctgcgcatcctgctgaacaacgccgccttccgcaacggccacaacttcatggtgcgcaacttccgctgcggccagcccctgcagaaccgggtgcagctgaagggccgcgacctgctgaccctgaagaacttcaccggcgaggagatccggtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctga

SEQ ID NO: 218 - DNA, 1068 bp, synthetic construct
  misc_feature 1..1068: note = Synthetic construct
  source 1..1068: mol_type = other DNA
atgctggtattcaacctgcgcatcctgctgaacaacgccgccttccgcaacggccacaacttcatggtgcgcaacttccgctgcggccagcccctgcagaaccgggtgcagctgaagggccgcgacctgctgaccctgaagaacttcaccggcgaggagatccggtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctga

SEQ ID NO: 219 - DNA, 1068 bp, synthetic construct; identical to SEQ ID NO: 216
  misc_feature 1..1068: note = Synthetic construct
  source 1..1068: mol_type = other DNA

SEQ ID NO: 220 - DNA, 1068 bp, synthetic construct; identical to SEQ ID NO: 217
  misc_feature 1..1068: note = Synthetic construct
  source 1..1068: mol_type = other DNA

SEQ ID NO: 221 - DNA, 1068 bp, synthetic construct; identical to SEQ ID NO: 218
  misc_feature 1..1068: note = Synthetic construct
  source 1..1068: mol_type = other DNA

SEQ ID NO: 222 - DNA, 1073 bp, synthetic construct
  misc_feature 1..1073: note = Synthetic construct
  source 1..1073: mol_type = other DNA
atgttgttcaacttgaggatcttgttgaacaacgccgccttcaggaacggacacaacttcatggtaaggaacttcaggtgcggacagcccttgcagaacaaagtacagttgaaaggaagggacttgttgacattgaaaaacttcacaggagaagaaatcaaatacatgttgtggttgtcggccgacttgaaattcaggatcaaacagaaaggagaatacttgcccttgttgcagggaaaatcgttgggaatgatcttcgaaaaaaggtcgacaaggacaaggttgtcgacagaaacaggattcgccttgttgggaggacacccctgcttcttgacaacacaggacatccacttgggagtaaacgaatcgttgacagacacagccagggtattgtcgtcgatggccgacgccgtattggccagggtatacaaacagtcggacttggacacattggccaaagaagcctcgatccccatcatcaacggattgtcggacttgtaccaccccatccagatcttggccgactacttgacattgcaggaacactactcgtcgttgaaaggattgacattgtcgtggatcggagacggaaacaacatcttgcactcgatcatgatgtcggccgccaaattcggaatgcacttgcaggccgccacacccaaaggatacgaacccgacgcctcggtaacaaaattggccgaacagtacgccaaagaaaacggaacaaaattgttgttgacaaacgaccccttggaagccgcccacggaggaaacgtattgatcacagacacatggatctcgatgggacaggaagaagaaaaaaaaaaaaggttgcaggccttccagggataccaggtaacaatgaaaacagccaaagtagccgcctcggactggacattcttgcactgcttgcccaggaaacccgaagaagtagacgacgaagtattctactcgcccaggtcgttggtattccccgaagccgaaaacaggaaatggacaatcatggccgtaatggtatcgttgttgacagactactcgccccagttgcagaaacccaaattctgaatagtgaa

SEQ ID NO: 223 - DNA, 1065 bp, synthetic construct
  misc_feature 1..1065: note = Synthetic construct
  source 1..1065: mol_type = other DNA
atgctgttcaacctgcgcatcctgctgaacaacgccgccttccgcaacggccacaacttcatggtgcgcaacttccgctgcggccagcccctgcagggcaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctga

SEQ ID NO: 224 - DNA, 1065 bp, synthetic construct
  misc_feature 1..1065: note = Synthetic construct
  source 1..1065: mol_type = other DNA
atgctgttcaacctgcgcatcctgctgaacaacgccgccttccgcaacggccacaacttcatggtgcgcaacttccgctgcggccagcccctgcagggccgggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctga

SEQ ID NO: 225 - DNA, 1065 bp, synthetic construct
  misc_feature 1..1065: note = Synthetic construct
  source 1..1065: mol_type = other DNA
atgctgttcaacctgcgcatcctgctgaacaacgccgccttccgcaacggccacaacttcatggtgcgcaacttccgctgcggccagcccctgcagggccgggtgcagctgaagggccgcgacctgctgaccctgaagaacttcaccggcgaggagatccggtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctga

SEQ ID NO: 226 - DNA, 1065 bp, synthetic construct; identical to SEQ ID NO: 223
  misc_feature 1..1065: note = Synthetic construct
  source 1..1065: mol_type = other DNA

SEQ ID NO: 227 - DNA, 1065 bp, synthetic construct; identical to SEQ ID NO: 175
  misc_feature 1..1065: note = Synthetic construct
  source 1..1065: mol_type = other DNA

SEQ ID NO: 228 - DNA, 1068 bp, synthetic construct; identical to SEQ ID NO: 218
  misc_feature 1..1068: note = Synthetic construct
  source 1..1068: mol_type = other DNA

SEQ ID NO: 229 - DNA, 1068 bp, synthetic construct; identical to SEQ ID NO: 218
  misc_feature 1..1068: note = Synthetic construct
  source 1..1068: mol_type = other DNA

SEQ ID NO: 230 - RNA, 21 nt, synthetic construct
  misc_feature 1..21: note = Synthetic construct
  source 1..21: mol_type = other RNA
aaccaatcgaaagaaaccaaa

SEQ ID NO: 231 - RNA, 21 nt, synthetic construct
  misc_feature 1..21: note = Synthetic construct
  source 1..21: mol_type = other RNA
ctctaatcaccaggagtaaaa

SEQ ID NO: 232 - RNA, 21 nt, synthetic construct
  misc_feature 1..21: note = Synthetic construct
  source 1..21: mol_type = other RNA
gagagagatcttaacaaaaaa

SEQ ID NO: 233 - RNA, 21 nt, synthetic construct
  misc_feature 1..21: note = Synthetic construct
  source 1..21: mol_type = other RNA
tgtgtaacaacaacaacaaca

SEQ ID NO: 234 - RNA, 21 nt, synthetic construct
  misc_feature 1..21: note = Synthetic construct
  source 1..21: mol_type = other RNA
ccgcagtaggaagagaaagcc

SEQ ID NO: 235 - RNA, 21 nt, synthetic construct
  misc_feature 1..21: note = Synthetic construct
  source 1..21: mol_type = other RNA
aaaaaaaaaagaaatcataaa

SEQ ID NO: 236 - RNA, 21 nt, synthetic construct
  misc_feature 1..21: note = Synthetic construct
  source 1..21: mol_type = other RNA
gagagaagaaagaagaagacg

SEQ ID NO: 237 - RNA, 21 nt, synthetic construct
  misc_feature 1..21: note = Synthetic construct
  source 1..21: mol_type = other RNA
caattaaaaatacttaccaaa

SEQ ID NO: 238 - RNA, 21 nt, synthetic construct
  misc_feature 1..21: note = Synthetic construct
  source 1..21: mol_type = other RNA
gcaaacagagtaagcgaaacg

SEQ ID NO: 239 - RNA, 21 nt, synthetic construct
  misc_feature 1..21: note = Synthetic construct
  source 1..21: mol_type = other RNA
gcgaagaagacgaacgcaaag

SEQ ID NO: 240 - RNA, 21 nt, synthetic construct
  misc_feature 1..21: note = Synthetic construct
  source 1..21: mol_type = other RNA
ttaggactgtattgactggcc

SEQ ID NO: 241 - RNA, 21 nt, synthetic construct
  misc_feature 1..21: note = Synthetic construct
  source 1..21: mol_type = other RNA
atcatcggaattcggaaaaag

SEQ ID NO: 242 - RNA, 21 nt, synthetic construct
  misc_feature 1..21: note = Synthetic construct
  source 1..21: mol_type = other RNA
aaaacaaaagttaaagcagac

SEQ ID NO: 243 - RNA, 21 nt, synthetic construct
  misc_feature 1..21: note = Synthetic construct
  source 1..21: mol_type = other RNA
tttatctcaaataagaaggca

SEQ ID NO: 244 - RNA, 21 nt, synthetic construct
  misc_feature 1..21: note = Synthetic construct
  source 1..21: mol_type = other RNA
ggtggggaggtgagatttctt

SEQ ID NO: 245 - RNA, 21 nt, synthetic construct
  misc_feature 1..21: note = Synthetic construct
  source 1..21: mol_type = other RNA
tgattaggaaactacaaagcc

SEQ ID NO: 246 - RNA, 21 nt, synthetic construct
  misc_feature 1..21: note = Synthetic construct
  source 1..21: mol_type = other RNA
catttttcaatttcataaaac

SEQ ID NO: 247 - RNA, 21 nt, synthetic construct
  misc_feature 1..21: note = Synthetic construct
  source 1..21: mol_type = other RNA
ttacttttaagcccaacaaaa

SEQ ID NO: 248 - RNA, 21 nt, synthetic construct
  misc_feature 1..21: note = Synthetic construct
  source 1..21: mol_type = other RNA
ggcgtgtgtgtgtgttgttga

SEQ ID NO: 249 - RNA, 21 nt, synthetic construct
  misc_feature 1..21: note = Synthetic construct
  source 1..21: mol_type = other RNA
gtggtgaaggggaaggtttag

SEQ ID NO: 250 - RNA, 21 nt, synthetic construct
  misc_feature 1..21: note = Synthetic construct
  source 1..21: mol_type = other RNA
ttgtttttttttggtttggtt

SEQ ID NO: 251 - RNA, 1331 nt, synthetic construct
  misc_feature 1..1331: note = Synthetic construct
  source 1..1331: mol_type = other RNA
aggattattacatcaaaacaaaaagccgccaccatgctggtattcaacctgcgcatcctgctgaacaacgccgccttccgcaacggccacaacttcatggtgcgcaacttccgctgcggccagcccctgcagaaccgggtgcagctgaagggccgcgacctgctgaccctgaagaacttcaccggcgaggagatccggtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaggtctctagtaatgagctggagcctcggtagccgttcctcctgcccgctgggcctcccaacgggccctcctcccctccttgcaccggcccttcctggtctttgaataaagtctgagtgggcagcatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 252 - RNA, 1334 nt, synthetic construct
  misc_feature 1..1334: note = Synthetic construct
  source 1..1334: mol_type = other RNA
aggattattacatcaaaacaaaaagccgccaccatgctggtattcaacctgcgcatcctgctgaacaacgccgccttccgcaacggccacaacttcatggtgcgcaacttccgctgcggccagcccctgcagaaccgggtgcagctgaagggccgcgacctgctgaccctgaagaacttcaccggcgaggagatccggtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaggtctctagtaatgagctggagcctcggtagccgttcctcctgcccgctgggcctcccaacgggccctcctcccctccttgcaccggcccttcctggtctttgaataaagtctgagtgggcagcatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagct

SEQ ID NO: 253 - RNA, 1328 nt, synthetic construct
  misc_feature 1..1328: note = Synthetic construct
  source 1..1328: mol_type = other RNA
aggattattacatcaaaacaaaaagccgccaccatgctgttcaacctgcgcatcctgctgaacaacgccgccttccgcaacggccacaacttcatggtgcgcaacttccgctgcggccagcccctgcagaacaaggtgcagctgaagggccgcgacctgctgaccctgaagaacttcaccggcgaggagatcaagtacatgctgtggctgagcgccgacctgaagttccgcatcaagcagaagggcgagtacctgcccctgctgcagggcaagagcctgggcatgatcttcgagaagcgcagcacccgcacccgcctgagcaccgagacaggcttcgccctgctgggcggccacccctgcttcctgaccacccaggacatccacctgggcgtgaacgagagcctgaccgacaccgcccgcgtgctgagcagcatggccgacgccgtgctggcccgcgtgtacaagcagagcgacctggacaccctggccaaggaggccagcatccccatcatcaacggcctgagcgacctgtaccaccccatccagatcctggccgactacctgaccctgcaggagcactacagcagcctgaagggcctgaccctgagctggatcggcgacggcaacaacatcctgcacagcatcatgatgagcgccgccaagttcggcatgcacctgcaggccgccacccccaagggctacgagcccgacgccagcgtgaccaagctggccgagcagtacgccaaggagaacggcaccaagctgctgctgaccaacgaccccctggaggccgcccacggcggcaacgtgctgatcaccgacacctggatcagcatgggccaggaggaggagaagaagaagcgcctgcaggccttccagggctaccaggtgaccatgaagaccgccaaggtggccgccagcgactggaccttcctgcactgcctgccccgcaagcccgaggaggtggacgacgaggtgttctacagcccccgcagcctggtgttccccgaggccgagaaccgcaagtggaccatcatggccgtgatggtgagcctgctgaccgactacagcccccagctgcagaagcccaagttctgaggtctctagtaatgagctggagcctcggtagccgttcctcctgcccgctgggcctcccaacgggccctcctcccctccttgcaccggcccttcctggtctttgaataaagtctgagtgggcagcatctagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2026/0078356 A1
(43) Pub. Date: Mar. 19, 2026

(54) COMPOSITIONS AND METHODS FOR TREATING ORNITHINE TRANSCARBAMYLASE DEFICIENCY

(71) Applicant: Arcturus Therapeutics, Inc., San Diego, CA (US)

(72) Inventors: Carlos G. PEREZ-GARCIA, San Diego, CA (US); Kiyoshi TACHIKAWA, San Diego, CA (US); Daiki MATSUDA, San Diego, CA (US); Padmanabh CHIVUKULA, San Diego, CA (US)

(21) Appl. No.: 19/279,738

(22) Filed: Jul. 24, 2025

Related U.S. Application Data
(63) Continuation of application No. 18/319,977, filed on May 18, 2023, now Pat. No. 12,398,379.
Application No. 18/319,977 is a continuation of application No. 16/705,102, filed on Dec. 5, 2019, now Pat. No. 11,685,906.
(60) Provisional application No. 62/776,302, filed on Dec. 6, 2018.

(51) Int. Cl. C12N 9/10; A61K 48/00; A61P 9/00

(52) U.S. Cl. CPC C12N 9/1018; A61P 9/00; C12Y 201/03003; A61K 48/00

(57) ABSTRACT

The present disclosure provides a modified human OTC protein having improved properties for the treatment of OTC deficiency in a patient. Preferably, the protein of the disclosure is produced from a codon optimized mRNA suitable for administration to a patient suffering from OTC deficiency wherein upon administration of the mRNA to the patient, the protein of the disclosure is expressed in the patient in therapeutically effective amounts to treat OTC deficiency. The present disclosure also provides codon optimized mRNA sequences encoding wild type human OTC comprising a 5′ UTR derived from a gene expressed by Arabidopsis thaliana for use in treating OTC deficiency in a patient.

### CROSS-REFERENCE TO RELATED APPLICATIONS

This application is a continuation of U.S. application Ser. No. 18/319,977, filed May 18, 2023, which is a continuation of U.S. application Ser. No. 16/705,102, filed Dec. 5, 2019, which claims the benefit of and priority to U.S. Provisional Application No. 62/776,302, filed Dec. 6, 2018. The contents of each application are incorporated herein by reference in their entirety.

### SEQUENCE LISTING

The instant application contains a Sequence Listing which has been submitted electronically in XML format and is hereby incorporated by reference in its entirety. Said XML copy, created on May 11, 2023 is named 049386-523C01US_SL_ST26.xml and is 568,374 bytes in size.

### BACKGROUND

Ornithine transcarbamylase (OTC) is a mitochondrial enzyme present in mammals which plays an essential role in detoxifying the organism from toxic ammonia. OTC mRNA has a mitochondrial signaling peptide (MSP) that is critical to redirect the nascent pre-peptide from the cytosol into the mitochondria. OTC protein exist as a precursor in the cytosol, the presence of the MSP redirects the pro-peptide into the mitochondria, where it undergoes excision of the signaling peptide and delivery of the functional protein into the mitochondrial matrix. OTC is one of six enzymes that play a role in the breakdown of proteins and removal of ammonia from the body, a process known as the urea cycle, a metabolic process that occurs in hepatocytes. OTC is responsible for converting carbamoyl phosphate and ornithine into citrulline.

Deficiency of the OTC enzyme results in excessive accumulation of nitrogen, in the form of ammonia (hyperammonemia), in the blood. Excess ammonia, which is a neurotoxin, travels to the central nervous system through the blood, resulting in the symptoms and physical findings associated with OTC deficiency. These symptoms can include vomiting, refusal to eat, progressive lethargy, or coma. If left untreated a hyperammonemic episode may progress to coma and life-threatening complications.

The severity and age of onset of OTC deficiency vary from person to person, even within the same family. A severe form of the disorder affects some infants, typically males, shortly after birth (neonatal period). A milder form of the disorder affects some children later in infancy. Both males and females may develop symptoms of OTC deficiency during childhood. Presently, the treatment of OTC deficiency is aimed at preventing excessive ammonia from being formed or from removing excessive ammonia during a hyperammonemic episode. Long-term therapy for OTC deficiency combines dietary restrictions and the stimulation of alternative methods of converting and excreting nitrogen from the body (alternative pathways therapy).

Dietary restrictions in individuals with OTC deficiency are aimed at limiting the amount of protein intake to avoid the development of excess ammonia. However, enough protein must be taken in by an affected infant to ensure proper growth. Infants with OTC deficiency are placed on a low protein, high calorie diet supplemented by essential amino acids.

In addition to dietary restrictions, individuals with OTC deficiency are treated by medications that stimulate the removal of nitrogen from the body. These medications provide an alternative method to the urea cycle in converting and removing nitrogen waste. These medications are unpalatable to many patients and are often administered via a tube that is placed in the stomach through the abdominal wall (gastrostomy tube) or a narrow tube that reaches the stomach via the nose (nasogastric tube).

In cases where there is no improvement or in cases where hyperammonemic coma develops, the removal of wastes by filtering an affected individual's blood through a machine (hemodialysis) may be necessary. Hemodialysis is also used to treat infants, children, and adults who are first diagnosed with OTC deficiency during hyperammonemic coma.

In some cases, liver transplantation, may be an appropriate treatment option. Liver transplantation can cure the hyperammonemia in OTC deficiency. However, this operation is risky and may result in post-operative complications. Also, after liver transplantation, patients will need to follow a medication regimen throughout their lives for immunosuppression.

Novel approaches and therapies are still needed for the treatment of OTC enzyme deficiency. Strategies are needed which overcome the challenges and limitations associated with, for example, gene therapy. Poor stability, getting enough OTC in the mitochondria and efficient delivery to the target cells are still challenges.

### SUMMARY

The present disclosure provides a modified human OTC protein of SEQ ID NO: 4 having improved properties for the treatment of OTC deficiency in a patient. SEQ ID NO: 4 has been modified from wild-type OTC to remove one or more predicted ubiquitination sites resulting in a protein that is less susceptible to ubiquitination and degradation by ubiquitin ligases. However, the modified OTC protein of SEQ ID NO: 4 maintains the catalytic enzyme activity of human wild type OTC. The removal of predicted ubiquitination sites preferably comprises replacing N-terminus residues that have been found to support ubiquitination such as asparagine, arginine, leucine, lysine or phenylalanine with N-terminus residues that have been found to be stabilizing against ubiquitination such as alanine, glycine, methionine, serine, threonine, valine and proline. Stabilization of the modified OTC protein of SEQ ID NO: 4 in this manner is particularly advantageous for preserving the stability of the modified OTC protein during its transport from the cytosol to the mitochondria wherein it exerts its enzymatic activity.

Preferably, the protein of SEQ ID NO: 4 described herein is produced from a nucleic acid encoding the protein of SEQ ID NO: 4. The nucleic acid may be RNA or DNA that encodes the protein of SEQ ID NO: 4. Preferably the nucleic acid is a heterologous mRNA construct comprising an open reading frame encoding for the modified protein of SEQ ID NO: 4. Preferably, the open reading frame is a codon-optimized open reading frame. Preferably, the open reading frame sequence is optimized to have a theoretical minimum of uridines possible to encode for the modified protein. Preferably, the heterologous mRNA construct comprises a 5′ cap, a 5′UTR, a 3′UTR, an open reading frame encoding a modified protein of SEQ ID NO: 4 and a 3′ poly A tail. Preferably, the 5′UTR derived from a gene expressed by Arabidopsis thaliana. Preferably the 5″ UTR derived from a gene expressed by Arabidopsis thaliana is found in Table 2.

The present disclosure also provides mRNA (also referred to herein as mRNA constructs or mRNA sequences) comprising an optimized coding region encoding wild type human ornithine transcarbamylase (OTC) protein of SEQ ID NO: 3 or an OTC protein sequence that is at least 95% identical over the full length of SEQ ID NO: 3 and having OTC protein enzymatic activity wherein the mRNA sequences comprise a 5′UTR derived from a gene expressed by Arabidopsis thaliana.

The mRNA constructs described herein provide high-efficiency expression of the OTC proteins described herein. The expression can be in vitro, ex vivo, or in vivo.

The present disclosure also provides pharmaceutical compositions comprising the mRNA sequences described herein and methods of treating omithine transcarbamylase (OTC) deficiency by administering the pharmaceutical compositions comprising the mRNA sequences described herein to a patient in need thereof wherein the OTC protein of SEQ ID NO: 3 or SEQ ID NO: 4 is expressed in the patient.

### BRIEF DESCRIPTION OF THE DRAWINGS

FIGS. 1A-1B show scatter plots illustrating ornithine transcarbamylase (OTC) protein expression in hepatocyte cell lines Hepa1,6 (mouse) and Hep3B (human) at 24 hours (FIG. 1A) and 48 hours (FIG. 1B) using In-Cell Western (ICW) assays.

FIGS. 2A-2B show scatter plots illustrating the correlation of protein stability compounds screened in Hepa1,6 cells (FIG. 2A) and Hep3B cells (FIG. 2B) at 24 h in Round 1.

FIGS. 3A-3B show scatter plots illustrating the correlation of protein stability compounds screened in human primary hepatocytes at 24 h and 48 h in Round 2 (newly optimized compounds based on Round 1) as shown with FIG. 3A and FIG. 3B.

FIGS. 4A-4B show scatter plots illustrating the correlation of protein stability compounds screened in human primary hepatocytes at 24 h and 48 h in Round 3 (newly optimized compounds based on rounds 1 and 2) as shown with FIG. 4A and FIG. 4B.

FIG. 5 is a plot illustrating OTC protein expression levels in human primary hepatocytes transfected with lead OTC mRNAs. 1799.7 is an mRNA having the sequence of SEQ ID NO: 175 wherein 100% of the uridines in SEQ ID NO: 175 are N¹-methylpseudouridine (N1MPU).

FIGS. 6A-6B show bar graphs depicting time course OTC expression levels in spf/ash mice dosed at 10 mg/kg with human-specific OTC-mRNA epitopes (FIG. 6A) or mouse-specific OTC-mRNA epitopes (FIG. 6B).

FIG. 7 is a bar graph depicting OTC expression levels in spf/ash mice dosed at 3 mg/kg with OTC-mRNAs using two different chemistries wherein 100% of the uridines are N¹-methylpseudouridine (N1MPU) and 100% of the uridines are 5-methoxyuridine (5MeOU).

FIG. 8 is a graph depicting OTC expression levels in Balb/c mice dosed with OTC-mRNAs at three different doses and using two different chemistries (N1MPU and 5MeOU).

FIG. 9 is a graph depicting urinary orotate levels measured in spf/ash mice dosed with OTC-mRNA 1799.7 at three different doses: 0.3 mg/kg, 1 mg/kg and 3 mg/kg. 1799.7 is an mRNA having the sequence of SEQ ID NO: 175 wherein 100% of the uridines in SEQ ID NO: 175 are 5MeOU.

FIG. 10 is a scatter plot comparing human OTC expression levels and Urinary Orotate at 96 h in spf/ash mice dosed with OTC-mRNAs at 1 mg/kg and 3 mg/kg using two different chemistries (N1MPU and 5MeOU).

FIG. 11 is a western blot illustrating the protein expression levels of OTC-mRNAs in spf/ash mice dosed at 1 mg/kg and 3 mg/kg with lead OTC-mRNAs.

FIG. 12 is a western blot illustrating the protein expression levels in mitochondrial vs cytosolic fractions of spf/ash mice treated with lead OTC-mRNAs.

FIG. 13 is a plot illustrating the time course of expression of urinary orotate levels in spf/ash mice treated with lead OTC-mRNA.

FIG. 14 is a plot illustrating the survival of OTC-deficient mice (spf/ash) on a high protein diet during treatment with three different doses of OTC-mRNA 1799.7.

FIG. 15 is a plot illustrating hOTC expression levels in male C57BL/6 mice dosed with OTC-mRNAs (2262) having different modifications.

### DETAILED DESCRIPTION

### Definitions

The term “omithine transcarbamylase” as used interchangeably herein with “OTC” or “hOTC”, or “OTC_HUMAN” generally refers to the human protein associated with UniPRotKB-P00480. The amino acid sequence for the wild type human OTC protein is represented herein by SEQ ID NO: 3.

The term “nucleic acid,” in its broadest sense, includes any compound and/or substance that comprise a polymer of nucleotides. These polymers are often referred to as polynucleotides. Exemplary nucleic acids or polynucleotides described herein include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof.

As used herein, the term “polynucleotide” is generally used to refer to a nucleic acid (e.g., DNA or RNA). When RNA, such as mRNA, is specifically being referred to, the term polyribonucleotide may be used. The terms polynucleotide, polyribonucleotide, nucleic acid, ribo nucleic acid, DNA, RNA, mRNA, and the like include such molecules that may be comprised of standard or unmodified residues; nonstandard or modified residues (e.g., analogs); and mixtures of standard and nonstandard (e.g., analogs) residues. In certain embodiments a polynucleotide or a polyribonucleotide is a modified polynucleotide or a polyribonucleotide. In the context of the present disclosure, for each RNA (polyribonucleotide) sequence listed herein, the corresponding DNA (polydeoxyribonucleotide or polynucleotide) sequence is contemplated and vice versa. “Polynucleotide” may be used interchangeably with the “oligomer”. Polynucleotide sequences shown herein are from left to right, 5′ to 3′, unless stated otherwise.

As used herein, the term “messenger RNA” (mRNA) refers to any polynucleotide which encodes a protein or polypeptide of interest and which is capable of being translated to produce the encoded protein or polypeptide of interest in vitro, in vivo, in situ or ex vivo.

As used herein, the term “translation” is the process in which ribosomes create polypeptides. In translation, messenger RNA (mRNA) is decoded by transfer RNAS (tRNAs) in a ribosome complex to produce a specific amino acid chain, or polypeptide. The coding region of a polynucleotide sequence (DNA or RNA), also known as the coding sequence or CDS, is capable of being converted to a protein or a fragment thereof by the process of translation.

As used herein, the term “codon-optimized” means a natural (or purposefully designed variant of a natural) coding sequence which has been redesigned by choosing different codons without altering the encoded protein amino acid sequence. Codon optimized sequence can increase the protein expression levels (Gustafsson et al., Codon bias and heterologous protein expression. 2004, Trends Biotechnol 22: 346-53) of the encoded proteins amongst providing other advantages. Variables such as high codon adaptation index (CAI), LowU method, mRNA secondary structures, cis-regulatory sequences, GC content and many other similar variables have been shown to somewhat correlate with protein expression levels (Villalobos et al., Gene Designer: a synthetic biology tool for constructing artificial DNA segments. 2006, BMC Bioinformatics 7:285). High CAI (codon adaptation index) method picks a most frequently used synonymous codon for an entire protein coding sequence. The most frequently used codon for each amino acid is deduced from 74,218 protein-coding genes from a human genome. The Low U method targets only U-containing codons that can be replaced with a synonymous codon with fewer U moieties. If there are a few choices for the replacement, the more frequently used codon will be selected. The remaining codons in the sequence are not changed by the Low U method. This method may be used in conjunction with the disclosed mRNAs to design coding sequences that are to be synthesized with, for example, 5-methoxyuridine or N¹-methylpseudouridine.

As used herein, “modified” refers to a change in the state or structure of a molecule disclosed herein. The molecule may be changed in many ways including chemically, structurally or functionally. Preferably a polynucleotide or polypeptide of the disclosure are modified as compared to the native form of the polynucleotide or polypeptide or as compared to a reference polypeptide sequence or polynucleotide sequence. For example, mRNA disclosed herein may be modified by codon optimization, or by the insertion of non-natural nucleosides or nucleotides. Polypeptides may be modified, for example, by site specific amino acid deletions or substitutions to alter the properties of the polypeptide.

As used herein, the term “homology” refers to the overall relatedness between polymeric molecules, e.g. between nucleic acid molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the present disclosure, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the present disclosure, two protein sequences are considered to be homologous if the proteins are at least about 50%, 60%, 70%, 80%, or 90% identical for at least one stretch of at least about 20 amino acids.

As used herein, the term “identity” refers to the overall relatedness between polymeric molecules, e.g., between oligonucleotide molecules (e.g. DNA molecules and/or RNA molecules) and/or between polypeptide molecules. Calculation of the percent identity of two polynucleotide sequences, for example, can be performed by aligning the two sequences for optimal comparison purposes. In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984), BLASTP, BLASTN, and FASTA Altschul, S. F. et al., J. Molec. Biol., 215, 403 (1990).

An “effective amount” of the mRNA sequence encoding an open reading frame (ORF) protein or a corresponding composition thereof is generally that amount of mRNA that provides efficient ORF protein production in a cell. Preferably protein production using an mRNA composition described herein is more efficient than a composition containing a corresponding wild type mRNA encoding an ORF protein. Increased efficiency may be demonstrated by increased cell transfection (i.e., the percentage of cells transfected with the nucleic acid), increased protein translation from the nucleic acid, decreased nucleic acid degradation (as demonstrated, e.g., by increased duration of protein translation from a modified nucleic acid), or reduced innate immune response of the host cell. When referring to an ORF protein described herein, an effective amount is that amount of ORF protein that overcomes an ORF protein deficiency in a cell.

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vivo” refers to events that occur within an organism (e.g., animal, plant, or microbe or cell or tissue thereof).

As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. Substantially isolated: By “substantially isolated” is meant that the compound is substantially separated from the environment in which it was formed or detected. Partial separation can include, for example, a composition enriched in the compound described herein. Substantial separation can include compositions containing at least about 50%, at least about 60%, at least about 70%, at least about 80%, at least about 90%, at least about 95%, at least about 97%, or at least about 99% by weight of the compound described herein, or salt thereof. Methods for isolating compounds and their salts are routine in the art.

As used herein, the term “subject” or “patient” refers to any organism to which a composition in accordance with the present disclosure may be administered, e.g., for experimental, diagnostic, prophylactic, and/or therapeutic purposes. Typical subjects include animals (e.g., mammals such as mice, rats, rabbits, non-human primates, and humans) and/or plants. Preferably “patient” refers to a human subject who may seek or be in need of treatment, requires treatment, is receiving treatment, will receive treatment, or a subject who is under care by a trained professional for a particular disease or condition.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

As used herein, the term “therapeutically effective amount” means an amount of an agent to be delivered (e.g., nucleic acid, protein or peptide, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

As used herein, a “total daily dose” is an amount given or prescribed in a 24 hr period. It may be administered as a single unit dose.

As used herein, the term “treating” refers to partially or completely alleviating, ameliorating, improving, relieving, delaying onset of, inhibiting progression of, reducing severity of, and/or reducing incidence of one or more symptoms or features of OTC deficiency. Treatment may be administered to a subject who does not exhibit signs of OTC deficiency and/or to a subject who exhibits only early signs of OTC deficiency for the purpose of decreasing the risk of developing pathology associated with the disease, disorder, and/or condition.

As used herein, the terms “transfect” or “transfection” mean the intracellular introduction of a nucleic acid into a cell, or preferably into a target cell. The introduced nucleic acid may be stably or transiently maintained in the target cell. The term “transfection efficiency” refers to the relative amount of nucleic acid up-taken by the target cell which is subject to transfection. In practice, transfection efficiency is estimated by the amount of a reporter nucleic acid product expressed by the target cells following transfection. Preferred are compositions with high transfection efficacies and in particular those compositions that minimize adverse effects which are mediated by transfection of non-target cells and tissues.

As used herein, the term “target cell” refers to a cell or tissue to which a composition of the disclosure is to be directed or targeted. In some embodiments, the target cells are deficient in a protein or enzyme of interest. For example, where it is desired to deliver a nucleic acid to a hepatocyte, the hepatocyte represents the target cell. In some embodiments, the nucleic acids and compositions of the present disclosure transfect the target cells on a discriminatory basis (i.e., do not transfect non-target cells). The compositions and methods of the present disclosure may be prepared to preferentially target a variety of target cells, which include, but are not limited to, hepatocytes, epithelial cells, hematopoietic cells, epithelial cells, endothelial cells, lung cells, bone cells, stem cells, mesenchymal cells, neural cells (e.g., meninges, astrocytes, motor neurons, cells of the dorsal root ganglia and anterior horn motor neurons), photoreceptor cells (e.g., rods and cones), retinal pigmented epithelial cells, secretory cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, reticulocytes, leukocytes, granulocytes and tumor cells.

Following transfection of one or more target cells by the compositions and nucleic acids described herein, expression of the protein encoded by such nucleic acid may be preferably stimulated and the capability of such target cells to express the protein of interest is enhanced. For example, transfection of a target cell with an OTC mRNA will allow expression of the OTC protein product following translation of the nucleic acid. The nucleic acids of the compositions and/or methods provided herein preferably encode a product (e.g., a protein, enzyme, polypeptide, peptide, functional RNA, and/or antisense molecule), and preferably encode a product whose in vivo production is desired.

As used herein “an OTC protein enzymatic activity” refers to enzyme activity that catalyzes the reaction between carbamoyl phosphate and omithine to form citrulline as part of the urea cycle in mammals.

As used herein, the term “about” or “approximately” as applied to one or more values of interest, refers to a value that is similar to a stated reference value. In certain embodiments, the term “approximately” or “about” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

### Polynucleotide Sequences

The present disclosure provides improved methods and compositions for the treatment of Ornithine transcarbamylase (OTC) deficiency using, for example, mRNA therapy. The present disclosure provides methods of treating ornithine transcarbamylase (OTC) deficiency, comprising administering to a subject in need of treatment a composition comprising an mRNA sequence described herein encoding a human omithine transcarbamylase (OTC) protein, modified forms of human OTC protein or active fragments of OTC protein at an effective dose and an administration interval such that at least one symptom or feature of the OTC deficiency is reduced in intensity, severity, or frequency or has delayed onset. The present disclosure also provides modified OTC proteins encoded by the mRNA sequences wherein the modified OTC proteins have improved properties such as enhanced stability and resistance to protein degradation and increased half-life as compared to wild type human OTC proteins.

Preferably, the administration of an mRNA composition described herein results in an increased OTC protein expression or activity of the subject as compared to a control level. Preferably, the control level is a baseline serum OTC protein expression or activity level in the subject prior to the treatment and/or the control level is indicative of the average serum OTC protein expression or activity level in OTC patients without treatment.

Preferably, administration of a mRNA described herein composition results in a reduced urinary orotic acid level in the subject as compared to a control orotic acid level. Preferably, the control orotic acid level is a baseline urinary orotic acid level in the subject prior to the treatment and/or the control orotic acid level is a reference level indicative of the average urinary orotic acid level in OTC patients without treatment.

Preferably, the OTC proteins encoded by the mRNA described herein are produced from a heterologous mRNA construct comprising an open reading frame (ORF) also referred to herein as a “coding sequence” (CDS) encoding for an OTC protein. Preferably, the coding sequence is codon-optimized. Preferably, coding sequence is optimized to have a theoretical minimum of uridines possible to encode for an OTC protein. Preferably, the mRNA constructs described herein comprise one or more of the following features: a 5′ cap; a 5′UTR, a 5′UTR enhancer sequence, a Kozak sequence or a partial Kozak sequence, a 3′UTR, an open reading frame encoding an OTC protein and a poly A tail. Preferably, the mRNA constructs described herein can provide high-efficiency expression of an OTC protein. The expression can be in vitro, ex vivo, or in vivo.

Preferably, a human OTC protein encoded by an mRNA described herein comprises a modified human OTC protein of SEQ ID NO: 4 shown in Table 1. SEQ ID NO: 4 has been modified from wild-type OTC of SEQ ID NO: 3 (Table 1) to remove one or more predicted ubiquitination sites resulting in a protein that is less susceptible to ubiquitination and degradation by ubiquitin ligases. The removal of predicted ubiquitination sites preferably comprises replacing N-terminus residues that have been found to support ubiquitination such as asparagine, arginine, leucine, lysine or phenylalanine with N-terminus residues that have been found to be stabilizing against ubiquitination such as alanine, glycine, methionine, serine, threonine, valine and proline. Stabilization of the modified OTC protein of SEQ ID NO: 4 in this manner is particularly advantageous for preserving the stability of the modified OTC protein during its transport from the cytosol to the mitochondria wherein it exerts its enzymatic activity.

Preferably, an OTC protein encoded by an mRNA described herein comprises a protein sequence that is at least 70%, 75%, 80%, 85%, 90%, 95%, 96%, 97%, 98%, 99%, or 100% identical to human wild type OTC protein of SEQ ID NO: 3 as shown in Table 1, while retaining the OTC protein activity of catalyzing the synthesis of citrulline (in the liver and small intestine) from carbamyl phosphate and ornithine.

TABLE 1
Selected OTC Nucleotide and Peptide Sequences

| | |
|---|---|
| mRNA coding | AUGCUGUUUAAUCUGAGGAUCCUGUUAAACAAUGCAGCUUUUAGAAA |
| sequence for wild | UGGUCACAACUUCAUGGUUCGAAAUUUUCGGUGUGGACAACCACUAC |
| type human OTC | AAAAUAAAGUGCAGCUGAAGGGCCGUGACCUUCUCACUCUAAAAAAC |
| | UUUACCGGAGAAGAAAUUAAAUAUAUGCUAUGGCUAUCAGCAGAUCU |
| | GAAAUUUAGGAUAAAACAGAAAGGAGAGUAUUUGCCUUUAUUGCAAG |
| | GGAAGUCCUUAGGCAUGAUUUUUGAGAAAAGAAGUACUCGAACAAGA |
| | UUGUCUACAGAAACAGGCUUUGCACUUCUGGGAGGACAUCCUUGUUU |
| | UCUUACCACACAAGAUAUUCAUUUGGGUGUGAAUGAAAGUCUCACGG |
| | ACACGGCCCGUGUAUUGUCUAGCAUGGCAGAUGCAGUAUUGGCUCGA |
| | GUGUAUAAACAAUCAGAUUUGGACACCCUGGCUAAAGAAGCAUCCAU |
| | CCCAAUUAUCAAUGGGCUGUCAGAUUUGUACCAUCCUAUCCAGAUCCU |
| | GGCUGAUUACCUCACGCUCCAGGAACACUAUAGCUCUCUGAAAGGUCU |
| | UACCCUCAGCUGGAUCGGGGAUGGGAACAAUAUCCUGCACUCCAUCAU |
| | GAUGAGCGCAGCGAAAUUCGGAAUGCACCUUCAGGCAGCUACUCCAAA |
| | GGGUUAUGAGCCGGAUGCUAGUGUAACCAAGUUGGCAGAGCAGUAUG |
| | CCAAAGAGAAUGGUACCAAGCUGUUGCUGACAAAUGAUCCAUUGGAA |
| | GCAGCGCAUGGAGGCAAUGUAUUAAUUACAGACACUUGGAUAAGCAU |
| | GGGACAAGAAGAGGAGAAGAAAAAGCGGCUCCAGGCUUUCCAAGGUU |
| | ACCAGGUUACAAUGAAGACUGCUAAAGUUGCUGCCUCUGACUGGACA |
| | UUUUUACACUGCUUGCCCAGAAAGCCAGAAGAAGUGGAUGAUGAAGU |
| | CUUUUAUUCUCCUCGAUCACUAGUGUUCCCAGAGGCAGAAAACAGAA |
| | AGUGGACAAUCAUGGCUGUCAUGGUGUCCCUGCUGACAGAUUACUCA |
| | CCUCAGCUCCAGAAGCCUAAAUUUUGA (SEQ ID NO: 1) |
| DNA coding sequence | ATGCTGTTTAATCTGAGGATCCTGTTAAACAATGCAGCTTTTAGAAATGG |
| for wild type human | TCACAACTTCATGGTTCGAAATTTTCGGTGTGGACAACCACTACAAAATA |
| OTC | AAGTGCAGCTGAAGGGCCGTGACCTTCTCACTCTAAAAAACTTTACCGG |
| | AGAAGAAATTAAATATATGCTATGGCTATCAGCAGATCTGAAATTTAGG |
| | ATAAAACAGAAAGGAGAGTATTTGCCTTTATTGCAAGGGAAGTCCTTAG |
| | GCATGATTTTTGAGAAAAGAAGTACTCGAACAAGATTGTCTACAGAAAC |
| | AGGCTTTGCACTTCTGGGAGGACATCCTTGTTTTCTTACCACACAAGATA |
| | TTCATTTGGGTGTGAATGAAAGTCTCACGGACACGGCCCGTGTATTGTCT |
| | AGCATGGCAGATGCAGTATTGGCTCGAGTGTATAAACAATCAGATTTGG |
| | ACACCCTGGCTAAAGAAGCATCCATCCCAATTATCAATGGGCTGTCAGA |
| | TTTGTACCATCCTATCCAGATCCTGGCTGATTACCTCACGCTCCAGGAAC |
| | ACTATAGCTCTCTGAAAGGTCTTACCCTCAGCTGGATCGGGGATGGGAA |
| | CAATATCCTGCACTCCATCATGATGAGCGCAGCGAAATTCGGAATGCAC |
| | CTTCAGGCAGCTACTCCAAAGGGTTATGAGCCGGATGCTAGTGTAACCA |
| | AGTTGGCAGAGCAGTATGCCAAAGAGAATGGTACCAAGCTGTTGCTGAC |
| | AAATGATCCATTGGAAGCAGCGCATGGAGGCAATGTATTAATTACAGAC |
| | ACTTGGATAAGCATGGGACAAGAAGAGGAGAAGAAAAAGCGGCTCCAG |
| | GCTTTCCAAGGTTACCAGGTTACAATGAAGACTGCTAAAGTTGCTGCCTC |
| | TGACTGGACATTTTTACACTGCTTGCCCAGAAAGCCAGAAGAAGTGGAT |
| | GATGAAGTCTTTTATTCTCCTCGATCACTAGTGTTCCCAGAGGCAGAAAA |
| | CAGAAAGTGGACAATCATGGCTGTCATGGTGTCCCTGCTGACAGATTAC |
| | TCACCTCAGCTCCAGAAGCCTAAATTTTGA (SEQ ID NO: 2) |
| Human wild type OTC | MLFNLRILLNNAAFRNGHNFMVRNFRCGQPLQNKVQLKGRDLLTLKNFTG |
| amino acid sequence | EEIKYMLWLSADLKFRIKQKGEYLPLLQGKSLGMIFEKRSTRTRLSTETGFA |
| (The signal peptide for | LLGGHPCFLTTQDIHLGVNESLTDTARVLSSMADAVLARVYKQSDLDTLAK |
| mitochondrial import | EASIPIFGLSDLYHPIQILADYLTLQEHYSSLKGLTLSWIGDGNNILHSIMMS |
| is underlined*) | AAKFGMHLQAATPKGYEPDASVTKLAEQYAKENGTKLLLTNDPLEAAHGG |
| | NVLITDTWISMGQEEEKKKRLQAFQGYQVTMKTAKVAASDWTFLHCLPRK |
| | PEEVDDEVFYSPRSLVFPEAENRKWTIMAVMVSLLTDYSPQLQKPKF |
| | (SEQ ID NO: 3) |
| Modified OTC amino | MLVFNLRILLNNAAFRNGHNFMVRNFRCGQPLQNRVQLKGRDLLTLKNFTGE |
| acid sequence | EIRYMLWLSADLKFRIKQKGEYLPLLQGKSLGMIFEKRSTRTRLSTETGFALLGGH |
| (The signal peptide for | PCFLTTQDIHLGVNESLTDTARVLSSMADAVLARVYKQSDLDTLAKEASIPIINGLS |
| mitochondrial import | DLYHPIQILADYLTLQEHYSSLKGLTLSWIGDGNNILHSIMMSAAKFGMHLQAAT |
| is underlined*) | PKGYEPDASVTKLAEQYAKENGTKLLLTNDPLEAAHGGNVLITDTWISMGQEEEK |
| | KKRLQAFQGYQVTMKTAKVAASDWTFLHCLPRKPEEVDDEVFYSPRSLVFPEAEN |
| | RKWTIMAVMVSLLTDYSPQLQKPKF (SEQ ID NO: 4) |

*The OTC protein comprises a signal peptide which is translated and which is responsible for translocation to the mitochondria. This signal peptide is represented by the first 32 amino acids as underlined in SEQ ID NO: 3 and SEQ ID NO: 4. The signal sequence of SEQ ID NO: 4 has also been modified as compared to SEQ ID NO: 3. An amino acid, valine is inserted at position 3 of SEQ ID NO: 4. This modification provides better mitochondrial localization of the modified OTC of SEQ ID NO: 4 as compared to wild type human OTC of SEQ ID NO: 3.

Preferably, the open reading frame (ORF) or coding sequence (CDS) of an mRNA sequence described herein encodes an amino acid sequence that is substantially identical to the modified OTC protein of SEQ ID NO: 4.

Preferably, the open reading frame (ORF) or coding sequence (CDS) of an mRNA sequence described herein encodes an amino acid sequence that is substantially identical to wild type human OTC protein of SEQ ID NO: 3. Preferably, an OTC protein encoded by an mRNA described herein comprises a protein sequence that is at least 70%, 75%, 80%, 85%, 90%, 95% 96%, 97%, 98%, 99%, or 100% identical to a modified human OTC protein of SEQ ID NO: 3 shown in Table 1 while retaining the OTC protein activity of catalyzing the synthesis of citrulline (in the liver and small intestine) from carbamyl phosphate and ornithine.

Preferably, the ORF or CDS of an mRNA described herein encodes an amino acid sequence that is substantially identical to modified human OTC protein of SEQ ID NO: 4.

Preferably, the ORF or CDS of an mRNA described herein encoding a human OTC protein comprises a codon optimized polynucleotide sequence at least 70%, 75%, 80%, 85%, 90%, 95%, 96%, 97%, 98%, 99%, or 100% identical to the mRNA coding sequence of SEQ ID NO: 1 of Table 1.

Preferably an mRNA described herein further comprises a sequence immediately downstream (i.e., in the 3′ direction from) of the CDS that creates a triple stop codon. The triple stop codon may be incorporated to enhance the efficiency of translation. In some embodiments, the translatable oligomer may comprise the sequence AUAAGUGAA (SEQ ID NO: 25) immediately downstream of an OTC CDS of an mRNA sequence described herein.

Preferably, an mRNA described herein further comprises a 5′ untranslated region (UTR) sequence. As is understood in the art, the 5′ and/or 3′ UTR may affect an mRNA's stability or efficiency of translation. The 5′ UTR may be derived from an mRNA molecule known in the art to be relatively stable (e.g., histone, tubulin, globin, glyceraldehyde 1-phosphate dehydrogenase (GAPDH), actin, or citric acid cycle enzymes) to increase the stability of the translatable oligomer. In other embodiments, a 5′ UTR sequence may include a partial sequence of a cytomegalovirus (CMV) immediate-early 1 (IE1) gene.

Preferably, the 5′ UTR comprises a sequence selected from the 5′ UTRs of human IL-6, alanine aminotransferase 1, human apolipoprotein E, human fibrinogen alpha chain, human transthyretin, human haptoglobin, human alpha-1-antichymotrypsin, human antithrombin, human alpha-1-antitrypsin, human albumin, human beta globin, human complement C3, human complement C5, SynK (thylakoid potassium channel protein derived from the cyanobacteria, Synechocystis sp.), mouse beta globin, mouse albumin, and a tobacco etch virus, or fragments of any of the foregoing. Preferably, the 5′ UTR is derived from a tobacco etch virus (TEV). Preferably, an mRNA described herein comprises a 5′ UTR sequence that is derived from a gene expressed by Arabidopsis thaliana. Preferably, the 5′ UTR sequence of a gene expressed by Arabidopsis thaliana is AT1G58420. Preferred 5′ UTR sequences comprise SEQ ID NOS: 5-10, 125-127 and 230-250: as shown in Table 2.

TABLE 2
5′UTR sequences

| Name | Sequence | Seq ID No.: |
|---|---|---|
| EV | UCAACACAACAUAUACAAAACAAACGAAUCUCAAGCAAUCAAGCA | SEQ ID NO: 5 |
| | UUCUACUUCUAUUGCAGCAAUUUAAAUCAUUUCUUUUAAAGCA | |
| | AAAGCAAUUUUCUGAAAAUUUUCACCAUUUACGAACGAUAG | |
| AT1G58420 | AUUAUUACAUCAAAACAAAAAGCCGCCA | SEQ ID NO: 6 |
| ARC5-2 | CUUAAGGGGGCGCUGCCUACGGAGGUGGCAGCCAUCUCCUUCU | SEQ ID NO: 7 |
| | CGGCAUCAAGCUUACCAUGGUGCCCCAGGCCCUGCUCUUGGUCC | |
| | CGCUGCUGGUGUUCCCCCUCUGCUUCGGCAAGUUCCCCAUCUAC | |
| | ACCAUCCCCGACAAGCUGGGGCCGUGGAGCCCCAUCGACAUCCA | |
| | CCACCUGUCCUGCCCCAACAACCUCGUGGUCGAGGACGAGGGCU | |
| | GCACCAACCUGAGCGGGUUCUCCUAC | |
| HCV | UGAGUGUCGU ACAGCCUCCA GGCCCCCCCC UCCCGGGAGA | SEQ ID NO: 8 |
| | GCCAUAGUGG UCUGCGGAACCGGUGAGUAC ACCGGAAUUG | |
| | CCGGGAAGAC UGGGUCCUUU CUUGGAUAAA | |
| | CCCACUCUAUGCCCGGCCAU UUGGGCGUGC CCCCGCAAGA | |
| | CUGCUAGCCG AGUAGUGUUG GGUUGCG | |
| HUMAN | AAUUAUUGGUUAAAGAAGUAUAUUAGUGCUAAUUUCCCUCCG | SEQ ID NO: 9 |
| ALBUMIN | UUUGUCCUAGCUUUUCUCUUCUGUCAACCCCACACGCCUUUGG | |
| | CACA | |
| EMCV | CUCCCUCCCC CCCCCCUAAC GUUACUGGCC GAAGCCGCUU | SEQ ID NO: 10 |
| | GGAAUAAGGC CGGUGUGCGU UUGUCUAUAU GUUAUUUUCC | |
| | ACCAUAUUGC CGUCUUUUGG CAAUGUGAGG GCCCGGAAAC | |
| | CUGGCCCUGU CUUCUUGACG AGCAUUCCUA GGGGUCUUUC | |
| | CCCUCUCGCC AAAGGAAUGC AAGGUCUGUU GAAUGUCGUG | |
| | AAGGAAGCAG UUCCUCUGGA AGCUUCUUGA AGACAAACAA | |
| | CGUCUGUAGC GACCCUUUGC AGGCAGCGGA ACCCCCCACC | |
| | UGGCGACAGG UGCCUCUGCG GCCAAAAGCC ACGUGUAUAA | |
| | GAUACACCUG CAAAGGCGGC ACAACCCCAG UGCCACGUUG | |
| | UGAGUUGGAU AGUUGUGGAA AGAGUCAAAU GGCUCUCCUC | |
| | AAGCGUAUUC AACAAGGGGC UGAAGGAUGC CCAGAAGGUA | |
| | CCCCAUUGUA UGGGAUCUGA UCUGGGGCCU CGGUGCACAU | |
| | GCUUUACGUG UGUUUAGUCG AGGUUAAAAA ACGUCUAGGC | |
| | CCCCCGAACC ACGGGGACGU GGUUUUCCUU UGAAAAACAC | |
| | GAUGAUAAU | |
| AT1G67090 | CACAAAGAGUAAAGAAGAACA | SEQ ID NO: |
| | | 125 |
| AT1G35720 | AACACUAAAAGUAGAAGAAAA | SEQ ID NO: |
| | | 126 |
| AT5G45900 | CUCAGAAAGAUAAGAUCAGCC | SEQ ID NO: |
| | | 127 |
| AT5G61250 | AACCAAUCGAAAGAAACCAAA | SEQ ID NO: |
| | | 230 |
| AT5G46430 | CUCUAAUCACCAGGAGUAAAA | SEQ ID NO: |
| | | 231 |
| AT5G47110 | GAGAGAGAUCUUAACAAAAAA | SEQ ID NO: |
| | | 232 |
| AT1G03110 | UGUGUAACAACAACAACAACA | SEQ ID NO: |
| | | 233 |
| AT3G12380 | CCGCAGUAGGAAGAGAAAGCC | SEQ ID NO: |
| | | 234 |
| AT5G45910 | AAAAAAAAAAGAAAUCAUAAA | SEQ ID NO: |
| | | 235 |
| AT1G07260 | GAGAGAAGAAAGAAGAAGACG | SEQ ID NO: |
| | | 236 |
| AT3G55500 | CAAUUAAAAAUACUUACCAAA | SEQ ID NO: |
| | | 237 |
| AT3G46230 | GCAAACAGAGUAAGCGAAACG | SEQ ID NO: |
| | | 238 |
| AT2G36170 | GCGAAGAAGACGAACGCAAAG | SEQ ID NO: |
| | | 239 |
| AT1G10660 | UUAGGACUGUAUUGACUGGCC | SEQ ID NO: |
| | | 240 |
| AT4G14340 | AUCAUCGGAAUUCGGAAAAAG | SEQ ID NO: |
| | | 241 |
| AT1G49310 | AAAACAAAAGUUAAAGCAGAC | SEQ ID NO: |
| | | 242 |
| AT4G14360 | UUUAUCUCAAAUAAGAAGGCA | SEQ ID NO: |
| | | 243 |
| AT1G28520 | GGUGGGGAGGUGAGAUUUCUU | SEQ ID NO: |
| | | 244 |
| AT1G20160 | UGAUUAGGAAACUACAAAGCC | SEQ ID NO: |
| | | 245 |
| AT5G37370 | CAUUUUUCAAUUUCAUAAAAC | SEQ ID NO: |
| | | 246 |
| AT4G11320 | UUACUUUUAAGCCCAACAAAA | SEQ ID NO: |
| | | 247 |
| AT5G40850 | GGCGUGUGUGUGUGUUGUUGA | SEQ ID NO: |
| | | 248 |
| AT1G06150 | GUGGUGAAGGGGAAGGUUUAG | SEQ ID NO: |
| | | 249 |
| AT2G26080 | UUGUUUUUUUUUGGUUUGGUU | SEQ ID |
| | | NO:250 |

Preferably the 5′UTR sequence comprises SEQ ID NO: 6 (AT1G58420).

Preferably, an mRNA described herein comprises a translation enhancer sequence. Translation enhancer sequences enhance the translation efficiency of a mRNA described herein and thereby provide increased production of the protein encoded by the mRNA. The translation enhancer region may be located in the 5′ or 3′ UTR of an mRNA sequence. Examples of translation enhancer regions include naturally-occurring enhancer regions from TEV 5′UTR and Xenopus beta-globin 3′UTR. Preferred 5′ UTR enhancer sequences include but are not limited to those derived from mRNAs encoding human heat shock proteins (HSP) including HSP70-P2, HSP70-M1 HSP72-M2, HSP17.9 and HSP70-P1. Preferred translation enhancer sequences used in accordance with the embodiments of the present disclosure are represented by SEQ ID NOS 11-15 as shown in Table 3.

TABLE 3
5′UTR enhancers

| Name | Sequence | Seq ID No.: |
|---|---|---|
| HSP70-P2 | GUCAGCUUUCAAACUCUUUGUUUCUUGUUUGUUGAUUGAGAAUA | SEQ ID NO: 11 |
| HSP70-M1 | CUCUCGCCUGAGAAAAAAAAUCCACGAACCAAUUUCUCAGCAACCAG | SEQ ID NO: 12 |
| | CAGCACG | |
| HSP72-M2 | ACCUGUGAGGGUUCGAAGGAAGUAGCAGUGUUUUUUGUUCCUAGA | SEQ ID NO: 13 |
| | GGAAGAG | |
| HSP17.9 | ACACAGAAACAUUCGCAAAAACAAAAUCCCAGUAUCAAAAUUCUUCU | SEQ ID NO: 14 |
| | CUUUUUUUCAUAUUUCGCAAAGAC | |
| HSP70-P1 | CAGAAAAAUUUGCUACAUUGUUUCACAAACUUCAAAUAUUAUUCAU | SEQ ID NO: 15 |
| | UUAUUU | |

Preferably, an mRNA described herein comprises a Kozak sequence. As is understood in the art, a Kozak sequence is a short consensus sequence centered around the translational initiation site of eukaryotic mRNAs that allows for efficient initiation of translation of the mRNA. The ribosomal translation machinery recognizes the AUG initiation codon in the context of the Kozak sequence. A Kozak sequence, may be inserted upstream of the coding sequence for OTC, downstream of a 5′ UTR or inserted upstream of the coding sequence for OTC and downstream of a 5′ UTR. Preferably, an mRNA described herein comprises a Kozak sequence having the amino acid sequence GCCACC (SEQ ID NO: 23). Preferably an mRNA described herein comprises a partial Kozak sequence “p” having the amino acid sequence GCCA (SEQ ID NO: 24).

Preferably an mRNA described herein comprises a 3′UTR. Preferably, the 3′ UTR comprises a sequence selected from the 3′ UTRs of alanine aminotransferase 1, human apolipoprotein E, human fibrinogen alpha chain, human haptoglobin, human antithrombin, human alpha globin, human beta globin, human complement C3, human growth factor, human hepcidin, MALAT-1, mouse beta globin, mouse albumin, and Xenopus beta globin, or fragments of any of the foregoing. Preferably, the 3′ UTR is derived from Xenopus beta globin. Preferred 3′ UTR sequences include SEQ ID NOS 16-22 as shown in Table 4.

TABLE 4
3′UTR sequences

| Name | Sequence | Seq ID No.: |
|---|---|---|
| XBG | CUAGUGACUGACUAGGAUCUGGUUACCACUAAACCAGCCU | SEQ ID NO: 16 |
| | CAAGAACACCCGAAUGGAGUCUCUAAGCUACAUAAUACCAA | |
| | CUUACACUUACAAAAUGUUGUCCCCCAAAAUGUAGCCAUUC | |
| | GUAUCUGCUCCUAAUAAAAAGAAAGUUUCUUCACAU | |
| HUMAN | UGCAAGGCUGGCCGGAAGCCCUUGCCUGAAAGCAAGAUUU | SEQ ID NO: 17 |
| HAPTOGLOBIN | CAGCCUGGAAGAGGGCAAAGUGGACGGGAGUGGACAGGAG | |
| | UGGAUGCGAUAAGAUGUGGUUUGAAGCUGAUGGGUGCCA | |
| | GCCCUGCAUUGCUGAGUCAAUCAAUAAAGAGCUUUCUUUU | |
| | GACCCAU | |
| HUMAN | ACGCCGAAGCCUGCAGCCAUGCGACCCCACGCCACCCCGUGC | SEQ ID NO: 18 |
| APOLIPOPROTEIN | CUCCUGCCUCCGCGCAGCCUGCAGCGGGAGACCCUGUCCCC | |
| E | GCCCCAGCCGUCCUCCUGGGGUGGACCCUAGUUUAAUAAA | |
| | GAUUCACCAAGUUUCACGCA | |
| HCV | UAGAGCGGCAAACCCUAGCUACACUCCAUAGCUAGUUUCU | SEQ ID NO: 19 |
| | UUUUUUUUUGUUUUUUUUUUUUUUUUUUUUUUUUUUU | |
| | UUUUUUUUUUUUCCUUUCUUUUCCUUUUUUUUUCCUCU | |
| | UUUCUUGGUGGCUCCAUCUUAGCCCUAGUCACGGCUAGCU | |
| | GUGAAAGGUCCGUGAGCCGCAUGACUGCAGAGAGUGCCGU | |
| | AACUGGUCUCUCUGCAGAUCAUGU | |
| MOUSE ALBUMIN | ACACAUCACAACCACAACCUUCUCAGGCUACCCUGAGAAAA | SEQ ID NO: 20 |
| | AAAGACAUGAAGACUCAGGACUCAUCUUUUCUGUUGGUGU | |
| | AAAAUCAACACCCUAAGGAACACAAAUUUCUUUAAACAUUU | |
| | GACUUCUUGUCUCUGUGCUGCAAUUAAUAAAAAAUGGAAA | |
| | GAAUCUAC | |
| HUMAN ALPHA | GCUGGAGCCUCGGUAGCCGUUCCUCCUGCCCGCUGGGCCU | SEQ ID NO: 21 |
| GLOBIN | CCCAACGGGCCCUCCUCCCCUCCUUGCACCGGCCCUUCCUG | |
| | GUCUUUGAAUAAAGUCUGAGUGGGCAGCA | |
| EMCV | UAGUGCAGUCAC UGGCACAACG CGUUGCCCGG | SEQ ID NO: 22 |
| | UAAGCCAAUC GGGUAUACAC | |
| | GGUCGUCAUACUGCAGACAG GGUUCUUCUA | |
| | CUUUGCAAGA UAGUCUAGAG UAGUAAAAUA | |
| | AAUAGUAUAAG | |

Preferably, an mRNA described herein comprises a 3′ tail region, which can serve to protect the mRNA from exonuclease degradation. The tail region may be a 3′poly(A) and/or 3′poly(C) region. Preferably, the tail region is a 3′ poly(A) tail. As used herein a “3′ poly(A) tail” is a polymer of sequential adenine nucleotides that can range in size from, for example: 10 to 250 sequential adenine nucleotides; 60-125 sequential adenine nucleotides, 90-125 sequential adenine nucleotides, 95-125 sequential adenine nucleotides, 95-121 sequential adenine nucleotides, 100 to 121 sequential adenine nucleotides, 110-121 sequential adenine nucleotides; sequential adenine nucleotides, 112-121 sequential adenine nucleotides; 114-121 adenine sequential nucleotides; and 115 to 121 sequential adenine nucleotides. Preferably a 3′ poly A tail as described herein comprise 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, or 125 sequential adenine nucleotides. 3′ Poly(A) tails can be added using a variety of methods known in the art, e.g., using poly(A) polymerase to add tails to synthetic or in vitro transcribed RNA. Other methods include the use of a transcription vector to encode poly A tails or the use of a ligase (e.g., via splint ligation using a T4 RNA ligase and/or T4 DNA ligase), wherein poly(A) may be ligated to the 3′ end of a sense RNA. Preferably, a combination of any of the above methods is utilized.

Preferably, an mRNA described herein comprises a 5′ cap. 5′-ends capped with various groups and their analogues are known in the art. The 5′ cap may be selected from m7GpppA, m7GpppC; unmethylated cap analogs (e.g., GpppG); dimethylated cap analog (e.g., m2,7GpppG), a trimethylated cap analog (e.g., m2,2,7GpppG), dimethylated symmetrical cap analogs (e.g., m7Gpppm7G), or anti reverse cap analogs (e.g., ARCA; m7, 2′OmeGpppG, m72′dGpppG, m7,3′OmeGpppG, m7,3′dGpppG and their tetraphosphate derivatives) (see, e.g., Jemielity, J. et al., RNA 9: 1108-1122 (2003). The 5′ cap may be an ARCA cap (3′-OMe-m7G(5′)pppG). The 5′ cap may be an mCAP (m7G(5′)ppp(5′)G, N⁷-Methyl-Guanosine-5′-Triphosphate-5′-Guanosine). The 5′ cap may be resistant to hydrolysis. A preferred 5′ cap is referred to herein as “m7GpppGm cap” also referred to herein as “Cap1” and has the following core structure:

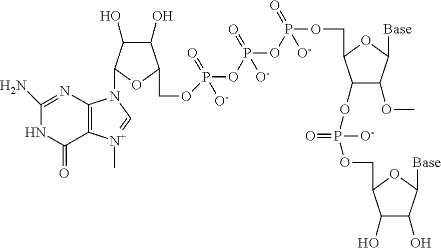

Preferably an mRNA described herein comprises one or more chemically modified nucleotides. Examples of nucleic acid monomers include non-natural, modified, and chemically-modified nucleotides, including any such nucleotides known in the art. mRNA sequences comprising chemically modified nucleotides have been shown to improve mRNA expression, expression rates, half-life and/or expressed protein concentrations. mRNA sequences comprising chemically modified nucleotides have also been useful to optimize protein localization thereby avoiding deleterious bio-responses such as the immune response and/or degradation pathways.

Examples of modified or chemically-modified nucleotides include 5-hydroxycytidines, 5-alkylcytidines, 5-hydroxyalkylcytidines, 5-carboxycytidines, 5-formylcytidines, 5-alkoxycytidines, 5-alkynylcytidines, 5-halocytidines, 2-thiocytidines, N⁴-alkylcytidines, N⁴-aminocytidines, N⁴-acetylcytidines, and N4,N⁴-dialkylcytidines.

Examples of modified or chemically-modified nucleotides include 5-hydroxycytidine, 5-methylcytidine, 5-hydroxymethylcytidine, 5-carboxycytidine, 5-formylcytidine, 5-methoxycytidine, 5-propynylcytidine, 5-bromocytidine, 5-iodocytidine, 2-thiocytidine; N⁴-methylcytidine, N⁴-aminocytidine, N⁴-acetylcytidine, and N4,N⁴-dimethylcytidine.

Examples of modified or chemically-modified nucleotides include 5-hydroxyuridines, 5-alkyluridines, 5-hydroxyalkyluridines, 5-carboxyuridines, 5-carboxyalkylesteruridines, 5-formyluridines, 5-alkoxyuridines, 5-alkynyluridines, 5-halouridines, 2-thiouridines, and 6-alkyluridines.

Examples of modified or chemically-modified nucleotides include 5-hydroxyuridine, 5-methyluridine, 5-hydroxymethyluridine, 5-carboxyuridine, 5-carboxymethylesteruridine, 5-formyluridine, 5-methoxyuridine (also referred to herein as “5MeOU”), 5-propynyluridine, 5-bromouridine, 5-fluorouridine, 5-iodouridine, 2-thiouridine, and 6-methyluridine.

Examples of modified or chemically-modified nucleotides include 5-methoxycarbonylmethyl-2-thiouridine, 5-methylaminomethyl-2-thiouridine, 5-carbamoylmethyluridine, 5-carbamoylmethyl-2′-O-methyluridine, 1-methyl-3-(3-amino-3-carboxypropy)pseudouridine, 5-methylaminomethyl-2-selenouridine, 5-carboxymethyluridine, 5-methyldihydrouridine, 5-taurinomethyluridine, 5-taurinomethyl-2-thiouridine, 5-(isopentenylaminomethyl)uridine, 2′-O-methylpseudouridine, 2-thio-2′O-methyluridine, and 3,2′-O-dimethyluridine.

Examples of modified or chemically-modified nucleotides include N⁶-methyladenosine, 2-aminoadenosine, 3-methyladenosine, 8-azaadenosine, 7-deazaadenosine, 8-oxoadenosine, 8-bromoadenosine, 2-methylthio-N⁶-methyladenosine, N⁶-isopentenyladenosine, 2-methylthio-N⁶-isopentenyladenosine, N⁶-(cis-hydroxyisopentenyl)adenosine, 2-methylthio-N⁶-(cis-hydroxyisopentenyl)adenosine, N⁶-glycinylcarbamoyladenosine, N6-threonylcarbamoyl-adenosine, N⁶-methyl-N⁶-threonylcarbamoyl-adenosine, 2-methylthio-N⁶-threonylcarbamoyl-adenosine, N⁶,N⁶-dimethyladenosine, N6-hydroxynorvalylcarbamoyladenosine, 2-methylthio-N⁶-hydroxynorvalylcarbamoyl-adenosine, N⁶-acetyl-adenosine, 7-methyl-adenine, 2-methylthio-adenine, 2-methoxy-adenine, alpha-thio-adenosine, 2′-O-methyl-adenosine, N⁶,2′-O-dimethyl-adenosine, N⁶,N6,2′-O-trimethyl-adenosine, 1,2′-O-dimethyl-adenosine, 2′-O-ribosyladenosine, 2-amino-N⁶-methyl-purine, 1-thio-adenosine, 2′-F-ara-adenosine, 2′-F-adenosine, 2′-OH-ara-adenosine, and N⁶-(19-amino-pentaoxanonadecyl)-adenosine.

Examples of modified or chemically-modified nucleotides include N¹-alkylguanosines, N²-alkylguanosines, thienoguanosines, 7-deazaguanosines, 8-oxoguanosines, 8-bromoguanosines, O⁶-alkylguanosines, xanthosines, inosines, and N¹-alkylinosines.

Examples of modified or chemically-modified nucleotides include N¹-methylguanosine, N²-methylguanosine, thienoguanosine, 7-deazaguanosine, 8-oxoguanosine, 8-bromoguanosine, O⁶-methylguanosine, xanthosine, inosine, and N¹-methylinosine.

Examples of modified or chemically-modified nucleotides include pseudouridines. Examples of pseudouridines include N¹-alkylpseudouridines, N¹-cycloalkylpseudouridines, N¹-hydroxypseudouridines, N¹-hydroxyalkylpseudouridines, N¹-phenylpseudouridines, N¹-phenylalkylpseudouridines, N¹-aminoalkylpseudouridines, N³-alkylpseudouridines, N⁶-alkylpseudouridines, N⁶-alkoxypseudouridines, N⁶-hydroxypseudouridines, N⁶-hydroxyalkylpseudouridines, N⁶-morpholinopseudouridines, N⁶-phenylpseudouridines, and N⁶-halopseudouridines. Examples of pseudouridines include N¹-alkyl-N⁶-alkylpseudouridines, N¹-alkyl-N⁶-alkoxypseudouridines, N¹-alkyl-N⁶-hydroxypseudouridines, N¹-alkyl-N⁶-hydroxyalkylpseudouridines, N¹-alkyl-N⁶-morpholinopseudouridines, N¹-alkyl-N⁶-phenylpseudouridines, and N¹-alkyl-N⁶-halopseudouridines. In these examples, the alkyl, cycloalkyl, and phenyl substituents may be unsubstituted, or further substituted with alkyl, halo, haloalkyl, amino, or nitro substituents.

Examples of pseudouridines include N¹-methylpseudouridine (also referred to herein as “N1MPU”), N¹-ethylpseudouridine, N¹-propylpseudouridine, N¹-cyclopropylpseudouridine, N¹-phenylpseudouridine, N¹-aminomethylpseudouridine, N³-methylpseudouridine, N¹-hydroxypseudouridine, and N¹-hydroxymethylpseudouridine.

Examples of nucleic acid monomers include modified and chemically-modified nucleotides, including any such nucleotides known in the art.

Examples of modified and chemically-modified nucleotide monomers include any such nucleotides known in the art, for example, 2′-O-methyl ribonucleotides, 2′-O-methyl purine nucleotides, 2′-deoxy-2′-fluoro ribonucleotides, 2′-deoxy-2′-fluoro pyrimidine nucleotides, 2′-deoxy ribonucleotides, 2′-deoxy purine nucleotides, universal base nucleotides, 5-C-methyl-nucleotides, and inverted deoxyabasic monomer residues.

Examples of modified and chemically-modified nucleotide monomers include 3′-end stabilized nucleotides, 3′-glyceryl nucleotides, 3′-inverted abasic nucleotides, and 3′-inverted thymidine.

Examples of modified and chemically-modified nucleotide monomers include locked nucleic acid nucleotides (LNA), 2′-0,4′-C-methylene-(D-ribofuranosyl) nucleotides, 2′-methoxyethoxy (MOE) nucleotides, 2′-methyl-thio-ethyl, 2′-deoxy-2′-fluoro nucleotides, and 2′-O-methyl nucleotides. In an exemplary embodiment, the modified monomer is a locked nucleic acid nucleotide (LNA).

Examples of modified and chemically-modified nucleotide monomers include 2′,4′-constrained 2′-O-methoxyethyl (cMOE) and 2′-O-Ethyl (cEt) modified DNAs.

Examples of modified and chemically-modified nucleotide monomers include 2′-amino nucleotides, 2′-O-amino nucleotides, 2′-C-allyl nucleotides, and 2′-O-allyl nucleotides.

Examples of modified and chemically-modified nucleotide monomers include N6-methyladenosine nucleotides.

Examples of modified and chemically-modified nucleotide monomers include nucleotide monomers with modified bases 5-(3-amino)propyluridine, 5-(2-mercapto)ethyluridine, 5-bromouridine; 8-bromoguanosine, or 7-deazaadenosine.

Examples of modified and chemically-modified nucleotide monomers include 2′-O-aminopropyl substituted nucleotides.

Examples of modified and chemically-modified nucleotide monomers include replacing the 2′—OH group of a nucleotide with a 2′-R, a 2′-OR, a 2′-halogen, a 2′-SR, or a 2′-amino, where R can be H, alkyl, alkenyl, or alkynyl.

Example of base modifications described above can be combined with additional modifications of nucleoside or nucleotide structure, including sugar modifications and linkage modifications. Certain modified or chemically-modified nucleotide monomers may be found in nature.

Preferred nucleotide modifications include N¹-methylpseudouridine and 5-methoxyuridine.

The constructs for preferred mRNA sequences are provided in Table 5.

TABLE 5
Exemplary mRNA Constructs

| mRNA | | | | | | | mRNA |
|---|---|---|---|---|---|---|---|
| Construct | | | | OTC Protein | | | Construct |
| No. | Cap | 5′UTR | Kozak* | Encoded | 3′UTR | 3′ Poly A Tail | SEQ ID NO: |
| 563 | Cap1 | TEV | Yes | SEQ ID NO: 3 | XBG | Yes | 26 |
| 564 | Cap1 | TEV | Yes | SEQ ID NO: 3 | XBG | Yes | 27 |
| 565 | Cap1 | TEV | Yes | SEQ ID NO: 3 | XBG | Yes | 28 |
| 566 | Cap1 | TEV | Yes | SEQ ID NO: 3 | XBG | Yes | 29 |
| 567 | Cap1 | TEV | Yes | SEQ ID NO: 3 | XBG | Yes | 30 |
| 568 | Cap1 | TEV | Yes | SEQ ID NO: 3 | XBG | Yes | 31 |
| 569 | Cap1 | TEV | Yes | SEQ ID NO: 3 | XBG | Yes | 32 |
| 570 | Cap1 | TEV | Yes | SEQ ID NO: 3 | XBG | Yes | 33 |
| 571 | Cap1 | TEV | Yes | SEQ ID NO: 3 | XBG | Yes | 34 |
| 572 | Cap1 | TEV | Yes | SEQ ID NO: 3 | XBG | Yes | 35 |
| 573 | Cap1 | TEV | Yes | SEQ ID NO: 3 | XBG | Yes | 36 |
| 574 | Cap1 | TEV | Yes | SEQ ID NO: 3 | XBG | Yes | 37 |
| 575 | Cap1 | TEV | P | SEQ ID NO: 3 | XBG | Yes | 38 |
| 708 | Cap1 | TEV | P | SEQ ID NO: 3 | XBG | Yes | 39 |
| 709 | Cap1 | TEV | P | SEQ ID NO: 3 | XBG | Yes | 40 |
| 710 | Cap1 | TEV | P | SEQ ID NO: 3 | XBG | Yes | 41 |
| 711 | Cap1 | TEV | P | SEQ ID NO: 3 | XBG | Yes | 42 |
| 712 | Cap1 | TEV | Yes | SEQ ID NO: 3 | XBG | Yes | 43 |
| 713 | Cap1 | TEV | Yes | SEQ ID NO: 3 | XBG | Yes | 44 |
| 714 | Cap1 | TEV | Yes | SEQ ID NO: 3 | XBG | Yes | 45 |
| 715 | Cap1 | TEV | Yes | SEQ ID NO: 3 | XBG | Yes | 46 |
| 716 | Cap1 | TEV | Yes | SEQ ID NO: 3 | XBG | Yes | 47 |
| 717 | Cap1 | TEV | Yes | SEQ ID NO: 3 | XBG | Yes | 48 |
| 718 | Cap1 | TEV | Yes | SEQ ID NO: 3 | XBG | Yes | 49 |
| 719 | Cap1 | TEV | Yes | SEQ ID NO: 3 | XBG | Yes | 50 |
| 720 | Cap1 | TEV | Yes | SEQ ID NO: 3 | XBG | Yes | 51 |
| 721 | Cap1 | TEV | Yes | SEQ ID NO: 3 | XBG | Yes | 52 |
| 722 | Cap1 | TEV | Yes | SEQ ID NO: 3 | XBG | Yes | 53 |
| 723 | Cap1 | TEV | Yes | SEQ ID NO: 3 | XBG | Yes | 54 |
| 724 | Cap1 | TEV | Yes | SEQ ID NO: 3 | XBG | Yes | 55 |
| 725 | Cap1 | TEV | Yes | SEQ ID NO: 3 | XBG | Yes | 56 |
| 726 | Cap1 | TEV | Yes | SEQ ID NO: 3 | XBG | Yes | 57 |
| 727 | Cap1 | TEV | Yes | SEQ ID NO: 3 | XBG | Yes | 58 |
| 728 | Cap1 | TEV | Yes | SEQ ID NO: 3 | XBG | Yes | 59 |
| 729 | Cap1 | TEV | Yes | SEQ ID NO: 3 | XBG | Yes | 60 |
| 1787 | Cap1 | ARC5-2 | No | SEQ ID NO: 3 | Hu haptoglob | Yes | 61 |
| 1788 | Cap1 | AT1G58420 | P | SEQ ID NO: 3 | Hu ApoE | Yes | 62 |
| 1789 | Cap1 | ARC5-2 | No | SEQ ID NO: 3 | Hu haptoglob | Yes | 63 |
| 1790 | Cap1 | AT1G58420 | P | SEQ ID NO: 3 | Hu ApoE | Yes | 64 |
| 1791 | Cap1 | ARC5-2 | No | SEQ ID NO: 3 | Hu haptoglob | Yes | 65 |
| 1792 | Cap1 | HCV5′ | P | SEQ ID NO: 3 | HCV3′ | Yes | 66 |
| 1793 | Cap1 | AT1G58420 | P | SEQ ID NO: 3 | Hu a-glob | Yes | 67 |
| 1794 | Cap1 | AT1G58420 | P | SEQ ID NO: 3 | Hu a-glob | Yes | 68 |
| 1795 | Cap1 | AT1G58420 | P | SEQ ID NO: 3 | Hu a-glob | Yes | 69 |
| 1796 | Cap1 | Hu alb | No | SEQ ID NO: 3 | Ms Alb | Yes | 70 |
| 1797 | Cap1 | Hu alb | No | SEQ ID NO: 3 | Ms Alb | Yes | 71 |
| 1798 | Cap1 | Hu alb | No | SEQ ID NO: 3 | Ms Alb | Yes | 72 |
| 1799 | Cap1 | AT1G58420 | Yes | SEQ ID NO: 3 | Hu a-glob | Yes | 73 |
| 1800 | Cap1 | Hu alb | No | SEQ ID NO: 3 | Ms Alb | Yes | 74 |
| 1801 | Cap1 | AT1G58420 | P | SEQ ID NO: 3 | Hu ApoE | Yes | 75 |
| 1802 | Cap1 | ARC5-2 | No | SEQ ID NO: 3 | Hu haptoglob | Yes | 76 |
| 1803 | Cap1 | TEV | Yes | SEQ ID NO: 3 | XBG | Yes | 77 |
| 1804 | Cap1 | TEV | Yes | SEQ ID NO: 3 | XBG | Yes | 78 |
| 1805 | Cap1 | TEV | Yes | SEQ ID NO: 3 | XBG | Yes | 79 |
| 1806 | Cap1 | TEV | Yes | SEQ ID NO: 3 | XBG | Yes | 80 |
| 1808 | Cap1 | TEV | Yes | SEQ ID NO: 3 | XBG | Yes | 81 |
| 1809 | Cap1 | TEV | Yes | SEQ ID NO: 3 | XBG | Yes | 82 |
| 1816 | Cap1 | TEV | P | SEQ ID NO: 3 | XBG | Yes | 83 |
| 1822 | Cap1 | TEV | Yes | SEQ ID NO: 3 | XBG | Yes | 84 |
| 1823 | Cap1 | TEV | Yes | SEQ ID NO: 3 | XBG | Yes | 85 |
| 1840 | Cap1 | EMCV | No | SEQ ID NO: 3 | EMCV | Yes | 86 |
| 1841 | Cap1 | EMCV | No | SEQ ID NO: 3 | EMCV | Yes | 87 |
| 1842 | Cap1 | EMCV | No | SEQ ID NO: 3 | EMCV | Yes | 88 |
| 1843 | Cap1 | HSP70-P2- | Yes | SEQ ID NO: 3 | XBG | Yes | 89 |
| | | TEV | | | | | |
| 1844 | Cap1 | HSP70-M1- | Yes | SEQ ID NO: 3 | XBG | Yes | 90 |
| | | TEV | | | | | |
| 1845 | Cap1 | HSP70-M2- | Yes | SEQ ID NO: 3 | XBG | Yes | 91 |
| | | TEV | | | | | |
| 1846 | Cap1 | HSP17.9- | Yes | SEQ ID NO: 3 | XBG | Yes | 92 |
| | | TEV | | | | | |
| 1847 | Cap1 | HSP70-P1- | Yes | SEQ ID NO: 3 | XBG | Yes | 93 |
| | | TEV | | | | | |
| 1882 | Cap1 | TEV | Yes | SEQ ID NO: 3 | XBG | Yes | 94 |
| 1883 | Cap1 | TEV | Yes | SEQ ID NO: 3 | XBG | Yes | 95 |
| 1884 | Cap1 | TEV | Yes | SEQ ID NO: 3 | XBG | Yes | 96 |
| 1885 | Cap1 | TEV | Yes | SEQ ID NO: 3 | XBG | Yes | 97 |
| 1886 | Cap1 | TEV | Yes | SEQ ID NO: 3 | XBG | Yes | 98 |
| 1887 | Cap1 | TEV | Yes | SEQ ID NO: 3 | XBG | Yes | 99 |
| 1888 | Cap1 | TEV | Yes | SEQ ID NO: 3 | XBG | Yes | 100 |
| 1889 | Cap1 | TEV | Yes | SEQ ID NO: 3 | XBG | Yes | 101 |
| 1890 | Cap1 | TEV | Yes | SEQ ID NO: 3 | XBG | Yes | 102 |
| 1891 | Cap1 | TEV | Yes | SEQ ID NO: 3 | XBG | Yes | 103 |
| 1898 | Cap1 | TEV | Yes | SEQ ID NO: 3 | XBG | Yes | 104 |
| 1899 | Cap1 | TEV | Yes | SEQ ID NO: 3 | XBG | Yes | 105 |
| 1900 | Cap1 | TEV | Yes | SEQ ID NO: 3 | XBG | Yes | 106 |
| 1903 | Cap1 | TEV | P | SEQ ID NO: 3 | XBG | Yes | 107 |
| 1904 | Cap1 | TEV | P | SEQ ID NO: 3 | XBG | Yes | 108 |
| 1905 | Cap1 | TEV | P | SEQ ID NO: 3 | XBG | Yes | 109 |
| 1906 | Cap1 | TEV | P | SEQ ID NO: 3 | XBG | Yes | 110 |
| 1907 | Cap1 | TEV | P | SEQ ID NO: 3 | XBG | Yes | 111 |
| 1908 | Cap1 | TEV | P | SEQ ID NO: 3 | XBG | Yes | 112 |
| 1915 | Cap1 | HSP70-P1- | Yes | SEQ ID NO: 3 | Hu a-glob | Yes | 113 |
| | | AT1G58420 | | | | | |
| 1916 | Cap1 | HSP70-P1- | Yes | SEQ ID NO: 3 | Hu a-glob | Yes | 114 |
| | | AT1G58420 | | | | | |
| 1917 | Cap1 | HSP70-P1- | Yes | SEQ ID NO: 3 | Hu a-glob | Yes | 115 |
| | | AT1G58420 | | | | | |
| 1918 | Cap1 | HSP70-P1- | Yes | SEQ ID NO: 3 | Hu a-glob | Yes | 116 |
| | | AT1G58420 | | | | | |
| 1919 | Cap1 | AT1G58420 | Yes | SEQ ID NO: 3 | Hu a-glob | Yes | 117 |
| 1920 | Cap1 | AT1G58420 | Yes | SEQ ID NO: 3 | Hu a-glob | Yes | 118 |
| 1921 | Cap1 | AT1G58420 | Yes | SEQ ID NO: | Hu a-glob | Yes | 119 |
| | | | | 4** | | | |
| 1925 | Cap1 | TEV | Yes | SEQ ID NO: 3 | XBG | Yes | 120 |
| 1926 | Cap1 | AT1G58420 | Yes | SEQ ID NO: 3 | Hu a-glob | Yes | 121 |
| 1927 | Cap1 | AT1G58420 | Yes | SEQ ID NO: 3 | Hu a-glob | Yes | 122 |
| 1928 | Cap1 | AT1G58420 | Yes | SEQ ID NO: 3 | Hu a-glob | Yes | 123 |
| 1929 | Cap1 | HSP70-P1- | Yes | SEQ ID NO: 3 | Hu a-glob | Yes | 124 |
| | | AT1G58420 | | | | | |
| 2016 | Cap1 | AT1G58420 | Yes | SEQ ID NO: 3 | Hu a-glob | Yes | 253 |
| 2260 | Cap1 | AT1G58420 | Yes | SEQ ID NO: | Hu a-glob | Yes | 251 |
| | | | | 4** | | | |
| 2262 | Cap1 | AT1G58420 | Yes | SEQ IS NO: | Hu a-glob | Yes | 252 |
| | | | | 4** | | | |

*Kozak sequence defined as GCCACC (SEQ ID NO: 23). Partial (P) Kozak defined as GCCA (SEQ ID NO: 24).
**Construct encodes modified human OTC protein of SEQ ID NO: 4.

Preferred mRNA sequences include all of the mRNA sequences listed in Table 5. Preferred mRNA sequences include all of the mRNA sequences listed wherein, 0% to 100%, preferably 1% to 100%, preferably 25% to 100%, preferably 50% to 100% and preferably 75% to 100% of the uracil nucleotides of the mRNA sequences are modified. Preferably, 1% to 100% of the uracil nucleotides are N¹-methylpseudouridine or 5-methoxyuridine. Preferably 100% of the uracil nucleotides are N¹-methylpseudouridine. Preferably 100% of the uracil nucleotides are 5-methoxyuridine.

Preferred mRNA sequences comprise a 5′ cap, a 5′UTR that is derived from a gene expressed by Arabidopsis thaliana, an optional translation enhancer sequence, an optional Kozak sequence or partial Kozak sequence, a codon optimized coding sequence (CDS/ORF) coding for an OTC protein, a 3′ UTR and a poly A tail. Preferably the codon optimized CDS encodes a protein of SEQ ID NO: 3 or SEQ ID NO: 4. Preferably, the 5′ UTR that is derived from a gene expressed by Arabidopsis thaliana is selected from found in Table 5. Preferably, the 5′ UTR that is derived from a gene expressed by Arabidopsis thaliana is selected from the group consisting of: SEQ ID NO: 6, SEQ ID NOS: 125-127 and SEQ ID NOS: 227-247. Preferably the 5′ UTR sequence is AT1G58420 having the sequence of SEQ ID NO: 6. Preferably, the uracil content of the codon optimized sequence has been reduced with respect to the percentages of uracil content of SEQ ID NO: 1. Preferably, 0% to 100% of the uracil nucleotides of the mRNA sequences are modified. Preferably, 0% to 100% of the uracil nucleotides are N¹-methylpseudouridine or 5-methoxyuridine. Preferably 100% of the uracil nucleotides are N¹-methylpseudouridine. Preferably 100% of the uracil nucleotides are 5-methoxyuridine.

Preferred mRNA constructs comprise codon optimized coding sequences and a 5′ UTR from a gene expressed by Arabidopsis thaliana and are selected from: SEQ ID NOS: 62, 67, 68, 69, 73, 113-119, 121-127.

A preferred mRNA construct of the disclosure comprises mRNA construct 1921 (SEQ ID NO: 119) having an optimized ORF encoding the modified human OTC protein of SEQ ID NO: 4 and comprising a 3′ Poly A tail of 121 nucleotides. Another preferred mRNA construct comprises construct 2260 (SEQ ID NO: 251) encoding the modified human OTC protein of SEQ ID NO: 4 and comprising a 3′ Poly A tail of 100 nucleotides. Another preferred mRNA construct comprises construct 2262 (SEQ ID NO: 252) encoding the modified human OTC protein of SEQ ID NO: 4 and comprising a 3′ Poly A tail of 100 nucleotides.

A preferred mRNA sequence of the disclosure includes the mRNA construct 1799 (SEQ ID NO: 73) having a codon optimized ORF encoding wild type human OTC of SEQ ID NO: 3 and having a 3′ Poly A tail of 121 nucleotides. Another preferred mRNA construct of the disclosure includes the mRNA construct 2016 (SEQ ID NO: 253) having a codon optimized ORF encoding wild type human OTC of SEQ ID NO: 3 and comprising a 3′ Poly A tail of 100 nucleotides.

Preferably 100% of the uridine nucleotides of mRNA constructs 1799, 2016, 1921, 2260 and 2262, are N1-methylpseudouridine. Preferably 100% of the uracil nucleotides of mRNA constructs 1799, 2016, 1921, 2260 and 2262, are 5-methoxyuridine.

The mRNA for use in accordance with this disclosure can exhibit increased translation efficiency. As used herein, translation efficiency refers to a measure of the production of a protein or polypeptide by translation of an mRNA in accordance with the disclosure. Preferably, an mRNA of the disclosure can exhibit at least 2-fold, 3-fold, 5-fold, or 10-fold increased translation efficiency in vivo as compared to mRNA encoding SEQ ID NO: 3 or SEQ ID NO: 4 that is not codon optimized in accordance with the disclosure and/or that does not comprise the preferred UTRs of the disclosure. Preferably an mRNA of the disclosure can provide at least a 2-fold, 3-fold, 5-fold, or 10-fold increased polypeptide or protein level in vivo as compared to mRNA encoding SEQ ID NO: 3 or SEQ ID NO: 4 that is not codon optimized and/or does not comprise the preferred UTRs of the disclosure. Preferably, an mRNA of the disclosure can provide increased levels of a polypeptide or protein in vivo as compared to mRNA encoding SEQ ID NO: 3 or SEQ ID NO: 4 that is not codon optimized in accordance with the disclosure and/or that does not comprise the preferred UTRs of the disclosure. For example, the level of a polypeptide or protein can be increased by 10%, or 20%, or 30%, or 40%, or 50%, or more.

Preferably the mRNA of the disclosure can provide increased functional half-life in the cytoplasm of mammalian cells over mRNA encoding SEQ ID NO: 3 or SEQ ID NO: 4 that is not codon optimized in accordance with the disclosure and/or that does not comprise the preferred UTRs of the disclosure. The inventive translatable molecules can have increased half-life of activity as compared to mRNA encoding SEQ ID NO: 3 or SEQ ID NO: 4 that is not codon optimized in accordance with the disclosure and/or that does not comprise the preferred UTRs of the disclosure.

Preferably, the mRNA of the disclosure can reduce cellular innate immune response as compared to mRNA encoding SEQ ID NO: 3 or SEQ ID NO: 4 that is not codon optimized in accordance with the disclosure and/or that does not comprise the preferred UTRs of the disclosure.

Preferably, the mRNA of the disclosure can reduce the dose levels required for efficacious therapy as compared to mRNA encoding SEQ ID NO: 3 or SEQ ID NO: 4 that is not codon optimized in accordance with the disclosure and/or that does not comprise the preferred UTRs of the disclosure.

Design and Synthesis of the mRNA Sequences

mRNA for use in accordance with the disclosure may be prepared according to any available technique including, but not limited to chemical synthesis, in vitro transcription (IVT) or enzymatic or chemical cleavage of a longer precursor, etc. Methods of synthesizing RNAs are known in the art.

In some embodiments, mRNA is produced from a primary complementary DNA (cDNA) construct. The process of design and synthesis of the primary cDNA constructs described herein generally includes the steps of gene construction, mRNA production (either with or without modifications) and purification. In the IVT method, a target polynucleotide sequence encoding an OTC protein is first selected for incorporation into a vector which will be amplified to produce a cDNA template. Optionally, the target polynucleotide sequence and/or any flanking sequences may be codon optimized. The cDNA template is then used to produce mRNA through in vitro transcription (IVT). After production, the mRNA may undergo purification and clean-up processes. The steps of which are provided in more detail below.

The step of gene construction may include, but is not limited to gene synthesis, vector amplification, plasmid purification, plasmid linearization and clean-up, and cDNA template synthesis and clean-up. Once a human OTC protein (e.g. SEQ ID NO: 3 or SEQ ID NO: 4) is selected for production, a primary construct is designed. Within the primary construct, a first region of linked nucleosides encoding the polypeptide of interest may be constructed using an open reading frame (ORF) of a selected nucleic acid (DNA or RNA) transcript. The ORF may comprise the wild type ORF, an isoform, variant or a fragment thereof. As used herein, an “open reading frame” or “ORF” is meant to refer to a nucleic acid sequence (DNA or RNA) which is capable of encoding a polypeptide of interest. ORFs often begin with the start codon, ATG and end with a nonsense or termination codon or signal.

Further, nucleotide sequence of any region of the mRNA or DNA template may be codon optimized. Codon optimization methods are known in the art and may be useful in efforts to achieve one or more of several goals. These goals include to match codon frequencies in target and host organisms to ensure proper folding, to bias GC content to increase mRNA stability or reduce secondary structures, to minimize tandem repeat codons or base runs that may impair gene construction or expression, to customize transcriptional and translational control regions, to insert or remove protein trafficking sequences, to remove/add post translation modification sites in encoded protein (e.g. glycosylation sites), to add, remove or shuffle protein domains, to insert or delete restriction sites, to modify ribosome binding sites and mRNA degradation sites, to adjust translational rates to allow the various domains of the protein to fold properly, or to reduce or eliminate problematic secondary structures within the mRNA. Suitable codon optimization tools, algorithms and services are known in the art.

Preferably, the primary cDNA template may include reducing the occurrence or frequency of appearance of certain nucleotides in the template strand. For example, the occurrence of a nucleotide in a template may be reduced to a level below 25% of nucleotides in the template. In further examples, the occurrence of a nucleotide in a template may be reduced to a level below 20% of nucleotides in the template. In some examples, the occurrence of a nucleotide in a template may be reduced to a level below 16% of nucleotides in the template. Preferably, the occurrence of a nucleotide in a template may be reduced to a level below 15%, and preferably may be reduced to a level below 12% of nucleotides in the template.

For example, the present disclosure provides nucleic acids wherein with altered uracil content at least one codon in the wild-type sequence has been replaced with an alternative codon to generate a uracil-altered sequence. Altered uracil sequences can have at least one of the following properties:
(i) an increase or decrease in global uracil content (i.e., the percentage of uracil of the total nucleotide content in the nucleic acid of a section of the nucleic acid, e.g., the open reading frame); or,
(ii) an increase or decrease in local uracil content (i.e., changes in uracil content are limited to specific subsequences); or,
(iii) a change in uracil distribution without a change in the global uracil content; or,
(iv) a change in uracil clustering (e.g., number of clusters, location of clusters, or distance between clusters); or,
(v) combinations thereof.

Preferably, the percentage of uracil nucleobases in the nucleic acid sequence is reduced with respect to the percentage of uracil nucleobases in the wild-type nucleic acid sequence. For example, 30% of nucleobases may be uracil in the wild-type sequence but the nucleobases that are uracil are preferably lower than 15%, preferably lower than 12% and preferably lower than 10% of the nucleobases in the in the nucleic acid sequences of the disclosure. The percentage uracil content can be determined by dividing the number of uracil in a sequence by the total number of nucleotides and multiplying by 100.

Preferably, the percentage of uracil nucleobases in a subsequence of the nucleic acid sequence is reduced with respect to the percentage of uracil nucleobases in the corresponding subsequence of the wild-type sequence. For example, the wild-type sequence may have a 5′-end region (e.g., 30 codons) with a local uracil content of 30%, and the uracil content in that same region could be reduced to preferably 15% or lower, preferably 12% or lower and preferably 10% or lower in the nucleic acid sequences of the disclosure.

Preferably, codons in the nucleic acid sequence of the invention reduce or modify, for example, the number, size, location, or distribution of uracil clusters that could have deleterious effects on protein translation. Although lower uracil content is desirable, in certain aspects, the uracil content, and in particular the local uracil content, of some subsequences of the wild-type sequence can be greater than the wild-type sequence and still maintain beneficial features (e.g., increased expression).

Preferably, the uracil-modified sequence induces a lower Toll-Like Receptor (TLR) response when compared to the wild-type sequence. Several TLRs recognize and respond to nucleic acids. Double-stranded (ds)RNA, a frequent viral constituent, has been shown to activate TLR3. Single-stranded (ss)RNA activates TLR7. RNA oligonucleotides, for example RNA with phosphorothioate internucleotide linkages, are ligands of human TLR8. DNA containing unmethylated CpG motifs, characteristic of bacterial and viral DNA, activate TLR9.

As used herein, the term “TLR response” is defined as the recognition of single-stranded RNA by a TLR7 receptor, and preferably encompasses the degradation of the RNA and/or physiological responses caused by the recognition of the single-stranded RNA by the receptor. Methods to determine and quantify the binding of an RNA to a TLR7 are known in the art. Similarly, methods to determine whether an RNA has triggered a TLR7-mediated physiological response (e.g., cytokine secretion) are well known in the art. Preferably, a TLR response can be mediated by TLR3, TLR8, or TLR9 instead of TLR7. Suppression of TLR7-mediated response can be accomplished via nucleoside modification. RNA undergoes over a hundred different nucleoside modifications in nature. Human rRNA, for example, has ten times more pseudouracil (′P) and 25 times more 2′-O-methylated nucleosides than bacterial rRNA. Bacterial mRNA contains no nucleoside modifications, whereas mammalian mRNAs have modified nucleosides such as 5-methylcytidine (m5C), N6-methyladenosine (m6A), inosine and many 2′-O-methylated nucleosides in addition to N7-methylguanosine (m7G).

Preferably, the uracil content of polynucleotides disclosed herein and preferably polynucleotides encoding the modified OTC protein of SEQ ID NO: 4 is less than 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 90%, 80%, 70%, 60%, 5%, 4%, 3%, 2% or 1% of the total nucleobases in the sequence in the reference sequence. Preferably, the uracil content of polynucleotides disclosed herein and preferably polynucleotides encoding the modified OTC protein of SEQ ID NO: 4, is between about 5% and about 25%. Preferably, the uracil content of polynucleotides disclosed herein and preferably polynucleotides encoding the modified OTC protein of SEQ ID NO: 4 is about 15% and about 25%.

The cDNA templates may be transcribed to produce an mRNA sequence described herein using an in vitro transcription (IVT) system. The system typically comprises a transcription buffer, nucleotide triphosphates (NTPs), an RNase inhibitor and a polymerase. The NTPs may be selected from, but are not limited to, those described herein including natural and unnatural (modified) NTPs. The polymerase may be selected from, but is not limited to, T7 RNA polymerase, T3 RNA polymerase and mutant polymerases such as, but not limited to, polymerases able to incorporate modified nucleic acids.

The primary cDNA template or transcribed mRNA sequence may also undergo capping and/or tailing reactions. A capping reaction may be performed by methods known in the art to add a 5′ cap to the 5′ end of the primary construct. Methods for capping include, but are not limited to, using a Vaccinia Capping enzyme (New England Biolabs, Ipswich, Mass.) or CLEANCAP® technology (TriLink Biotechnologies). A poly-A tailing reaction may be performed by methods known in the art, such as, but not limited to, 2′ O-methyltransferase and by methods as described herein. If the primary construct generated from cDNA does not include a poly-T, it may be beneficial to perform the poly-A-tailing reaction before the primary construct is cleaned.

Codon optimized cDNA constructs encoding an omithine transcarbamylase (OTC) protein are particularly suitable for generating mRNA sequences described herein. For example, such cDNA constructs may be used as the basis to transcribe, in vitro, a polyribonucleotide encoding an omithine transcarbamylase (OTC) protein. Table 6 provides a listing of exemplary cDNA ORF templates used for in vitro transcription of the mRNA sequences listed in Table 5.

TABLE 6
Exemplary cDNA Templates

| | | Protein encoded by cDNA |
|---|---|---|
| DNA Construct No***: | SEQ ID NO: | template SEQ ID NO: |
| p563 | 128 | SEQ ID NO: 3* |
| p564 | 129 | SEQ ID NO: 3* |
| p565 | 130 | SEQ ID NO: 3* |
| p566 | 131 | SEQ ID NO: 3* |
| p567 | 132 | SEQ ID NO: 3* |
| p568 | 133 | SEQ ID NO: 3* |
| p569 | 134 | SEQ ID NO: 3* |
| p570 | 135 | SEQ ID NO: 3* |
| p571 | 136 | SEQ ID NO: 3* |
| p572 | 137 | SEQ ID NO: 3* |
| p573 | 138 | SEQ ID NO: 3* |
| p574 | 139 | SEQ ID NO: 3* |
| p575 | 140 | SEQ ID NO: 3* |
| p708 | 141 | SEQ ID NO: 3* |
| p709 | 142 | SEQ ID NO: 3* |
| p710 | 143 | SEQ ID NO: 3* |
| p711 | 144 | SEQ ID NO: 3* |
| p712 | 145 | SEQ ID NO: 3* |
| p713 | 146 | SEQ ID NO: 3* |
| p714 | 147 | SEQ ID NO: 3* |
| p715 | 148 | SEQ ID NO: 3* |
| p716 | 149 | SEQ ID NO: 3* |
| p717 | 150 | SEQ ID NO: 3* |
| p718 | 151 | SEQ ID NO: 3* |
| p719 | 152 | SEQ ID NO: 3* |
| p720 | 153 | SEQ ID NO: 3* |
| p721 | 154 | SEQ ID NO: 3* |
| p722 | 155 | SEQ ID NO: 3* |
| p723 | 156 | SEQ ID NO: 3* |
| p724 | 157 | SEQ ID NO: 3* |
| p725 | 158 | SEQ ID NO: 3* |
| p726 | 159 | SEQ ID NO: 3* |
| p727 | 160 | SEQ ID NO: 3* |
| p728 | 161 | SEQ ID NO: 3* |
| p729 | 162 | SEQ ID NO: 3* |
| p1787 | 163 | SEQ ID NO: 3* |
| p1788 | 164 | SEQ ID NO: 3* |
| p1789 | 165 | SEQ ID NO: 3* |
| p1790 | 166 | SEQ ID NO: 3* |
| p1791 | 167 | SEQ ID NO: 3* |
| p1792 | 168 | SEQ ID NO: 3* |
| p1793 | 169 | SEQ ID NO: 3* |
| p1794 | 170 | SEQ ID NO: 3* |
| p1795 | 171 | SEQ ID NO: 3* |
| p1796 | 172 | SEQ ID NO: 3* |
| p1797 | 173 | SEQ ID NO: 3* |
| p1798 | 174 | SEQ ID NO: 3* |
| p1799 | 175 | SEQ ID NO: 3* |
| p1800 | 176 | SEQ ID NO: 3* |
| p1801 | 177 | SEQ ID NO: 3* |
| p1802 | 178 | SEQ ID NO: 3* |
| p1803 | 179 | SEQ ID NO: 3* |
| p1804 | 180 | SEQ ID NO: 3* |
| p1805 | 181 | SEQ ID NO: 3* |
| p1806 | 182 | SEQ ID NO: 3* |
| p1808 | 183 | SEQ ID NO: 3* |
| p1809 | 184 | SEQ ID NO: 3* |
| p1816 | 185 | SEQ ID NO: 3* |
| p1822 | 186 | SEQ ID NO: 3* |
| p1823 | 187 | SEQ ID NO: 3* |
| p1840 | 188 | SEQ ID NO: 3* |
| p1841 | 189 | SEQ ID NO: 3* |
| p1842 | 190 | SEQ ID NO: 3* |
| p1843 | 191 | SEQ ID NO: 3* |
| p1844 | 192 | SEQ ID NO: 3* |
| p1845 | 193 | SEQ ID NO: 3* |
| p1846 | 194 | SEQ ID NO: 3* |
| p1847 | 195 | SEQ ID NO: 3* |
| p1882 | 196 | SEQ ID NO: 3* |
| p1883 | 197 | SEQ ID NO: 3* |
| p1884 | 198 | SEQ ID NO: 3* |
| p1885 | 199 | SEQ ID NO: 3* |
| p1886 | 200 | SEQ ID NO: 3* |
| p1887 | 201 | SEQ ID NO: 3* |
| p1888 | 202 | SEQ ID NO: 3* |
| p1889 | 203 | SEQ ID NO: 3* |
| p1890 | 204 | SEQ ID NO: 3* |
| p1891 | 205 | SEQ ID NO: 3* |
| p1898 | 206 | SEQ ID NO: 3* |
| p1899 | 207 | SEQ ID NO: 3* |
| p1900 | 208 | SEQ ID NO: 3* |
| p1903 | 209 | SEQ ID NO: 3* |
| p1904 | 210 | SEQ ID NO: 3* |
| p1905 | 211 | SEQ ID NO: 3* |
| p1906 | 212 | SEQ ID NO: 3* |
| p1907 | 213 | SEQ ID NO: 3* |
| p1908 | 214 | SEQ ID NO: 3* |
| p1915 | 215 | SEQ ID NO: 3* |
| p1916 | 216 | SEQ ID NO: 3* |
| p1917 | 217 | SEQ ID NO: 3* |
| p1918 | 218 | SEQ ID NO: 3* |
| p1919 | 219 | SEQ ID NO: 3* |
| p1920 | 220 | SEQ ID NO: 3* |
| p1921 | 221 | SEQ ID NO: 4** |
| p1925 | 222 | SEQ ID NO: 3* |
| p1926 | 223 | SEQ ID NO: 3* |
| p1927 | 224 | SEQ ID NO: 3* |
| p1928 | 225 | SEQ ID NO: 3* |
| p1929 | 226 | SEQ ID NO: 3* |
| p2016 | 175 | SEQ ID NO: 3* |
| p2260 | 221 | SEQ ID NO: 4** |
| p2262 | 221 | SEQ ID NO: 4** |

*SEQ ID NO: 3 is the amino acid sequence for wild type human OTC.
**SEQ ID NO: 4 is the amino acid sequence for modified human OTC.
***The entire plasmid sequence is not included.

Preferred cDNA template sequences include the DNA sequence of SEQ ID NO: 175 (p1779) having an optimized coding sequence encoding wild type human OTC of SEQ ID NO: 3. Preferred cDNA template sequences also include cDNA sequence of SEQ ID NO: 221 (p1921), having an optimized coding sequence encoding a modified OTC protein of SEQ ID NO: 4.

The present disclosure also provides polynucleotides (e.g. DNA, RNA, cDNA, mRNA) encoding a human OTC protein that may be operably linked to one or more regulatory nucleotide sequences in an expression construct, such as a vector or plasmid. In certain embodiments, such constructs are DNA constructs. Regulatory nucleotide sequences will generally be appropriate for a host cell used for expression. Numerous types of appropriate expression vectors and suitable regulatory sequences are known in the art for a variety of host cells.

Typically, said one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the embodiments of the present disclosure. The promoters may be either naturally occurring promoters, or hybrid promoters that combine elements of more than one promoter.

An expression construct may be present in a cell on an episome, such as a plasmid, or the expression construct may be inserted in a chromosome. Preferably, the expression vector contains a selectable marker gene to allow the selection of transformed host cells. Selectable marker genes are well known in the art and will vary with the host cell used.

The present disclosure also provides expression vectors comprising a nucleotide sequence encoding an ornithine transcarbamylase (OTC) protein that is preferably operably linked to at least one regulatory sequence. Regulatory sequences are art-recognized and are selected to direct expression of the encoded polypeptide.

Accordingly, the term regulatory sequence includes promoters, enhancers, and other expression control elements. The design of the expression vector may depend on such factors as the choice of the host cell to be transformed and/or the type of protein desired to be expressed.

The present disclosure also provides a host cell transfected with an mRNA or DNA described herein which encodes an ornithine transcarbamylase (OTC) polypeptide described herein. Preferably, the human OTC polypeptide has the sequence of SEQ ID NO: 4. The host cell may be any prokaryotic or eukaryotic cell. For example, an ornithine transcarbamylase (OTC) polypeptide may be expressed in bacterial cells such as E. coli, insect cells (e.g., using a baculovirus expression system), yeast, or mammalian cells. Other suitable host cells are known to those skilled in the art.

The present disclosure also provides a host cell comprising a vector comprising a polynucleotide which encodes an mRNA sequence of any one of SEQ ID NOs: 26-229.

The present disclosure also provides methods of producing a human wild type OTC protein of SEQ ID NO: 3 or a modified human OTC protein SEQ ID NO: 4. Preferably, the OTC protein is SEQ ID NO: 4 and is encoded by mRNA of SEQ ID NO 119. For example, a host cell transfected with an expression vector encoding an OTC protein can be cultured under appropriate conditions to allow expression of the polypeptide to occur. The polypeptide may be secreted and isolated from a mixture of cells and medium containing the polypeptides. Alternatively, the polypeptides may be retained in the cytoplasm or in a membrane fraction and the cells harvested, lysed and the protein isolated. A cell culture includes host cells, media and other byproducts. Suitable media for cell culture are well known in the art.

The expressed OTC proteins described herein can be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of the OTC polypeptide.

### Lipid-Based Formulations

Lipid-based formulations have been increasingly recognized as one of the most promising delivery systems (also referred to herein as a delivery vehicle or carrier) for RNA due to their biocompatibility and their ease of large-scale production. Cationic lipids have been widely studied as synthetic materials for delivery of RNA. After mixing together, nucleic acids are condensed by cationic lipids to form lipid/nucleic acid complexes known as lipoplexes. These lipid complexes are able to protect genetic material from the action of nucleases and to deliver it into cells by interacting with the negatively charged cell membrane. Lipoplexes can be prepared by directly mixing positively charged lipids at physiological pH with negatively charged nucleic acids.

Conventional liposomes consist of a lipid bilayer that can be composed of cationic, anionic, or neutral (phospho)lipids and cholesterol, which encloses an aqueous core. Both the lipid bilayer and the aqueous space can incorporate hydrophobic or hydrophilic compounds, respectively. Liposome characteristics and behavior in vivo can be modified by addition of a hydrophilic polymer coating, e.g. polyethylene glycol (PEG), to the liposome surface to confer steric stabilization. Furthermore, liposomes can be used for specific targeting by attaching ligands (e.g., antibodies, peptides, and carbohydrates) to its surface or to the terminal end of the attached PEG chains (Front Pharmacol. 2015 Dec. 1; 6:286).

Liposomes are colloidal lipid-based and surfactant-based delivery systems composed of a phospholipid bilayer surrounding an aqueous compartment. They may present as spherical vesicles and can range in size from 20 nm to a few microns. Cationic lipid-based liposomes are able to complex with negatively charged nucleic acids via electrostatic interactions, resulting in complexes that offer biocompatibility, low toxicity, and the possibility of the large-scale production required for in vivo clinical applications. Liposomes can fuse with the plasma membrane for uptake; once inside the cell, the liposomes are processed via the endocytic pathway and the genetic material is then released from the endosome/carrier into the cytoplasm. Liposomes have long been perceived as drug delivery vehicles because of their superior biocompatibility, given that liposomes are basically analogs of biological membranes, and can be prepared from both natural and synthetic phospholipids (Int J Nanomedicine. 2014; 9: 1833-1843).

Cationic liposomes have been traditionally the most commonly used non-viral delivery systems for oligonucleotides, including plasmid DNA, antisense oligos, and siRNA/small hairpin R A-shRNA). Cationic lipids, such as DOTAP, (1,2-dioleoyl-3-trimethylammonium-propane) and DOTMA (N-[1-(2,3-dioleoyloxy)propyl]-N,N,N-trimethyl-ammonium methyl sulfate) can form complexes or lipoplexes with negatively charged nucleic acids to form nanoparticles by electrostatic interaction, providing high in vitro transfection efficiency. Furthermore, neutral lipid-based nanoliposomes for RNA delivery as e.g. neutral 1,2-dioleoyl-sn-glycero-3-phosphatidylcholine (DOPC)-based nanoliposomes were developed. (Adv Drug Deliv. Rev. 2014 February; 66: 110-116.)

Preferably, the mRNA constructs described herein are lipid formulated. The lipid formulation is preferably selected from, but not limited to, liposomes, lipoplexes, copolymers, such as PLGA, and lipid nanoparticles. Preferably a lipid nanoparticle (LNP) comprises:
(a) a nucleic acid,
(b) a cationic lipid,
(c) an aggregation reducing agent (such as polyethylene glycol (PEG) lipid or PEG-modified lipid),
(d) optionally a non-cationic lipid (such as a neutral lipid), and
(e) optionally, a sterol.
   Preferably, the lipid nanoparticle formulation consists of (i) at least one cationic lipid; (ii) a neutral lipid; (iii) a sterol, e.g., cholesterol; and (iv) a PEG-lipid, in a molar ratio of about 20-60% cationic lipid: 5-25% neutral lipid: 25-55% sterol; 0.5-15% PEG-lipid.

Some examples of lipids and lipid compositions for delivery of an active molecule of this disclosure are given in WO 2015/074085, U.S. 2018/0169268, WO 2018/119163, WO 20185/118102, U.S. 2018/0222863, WO 2016/081029, WO 2017/023817, WO 2017/117530, each of which is hereby incorporated by reference in its entirety. In certain embodiments, the lipid is a compound of the following Formula I:

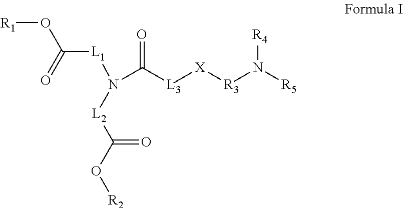

wherein
R₁ and R₂ both consist of a linear alkyl consisting of 1 to 14 carbons, or an alkenyl or alkynyl consisting of 2 to 14 carbons;
L₁ and L₂ both consist of a linear alkylene or alkenylene consisting of 5 to 18 carbons, or forming a heterocycle with N;
X is S;
L₃ consists of a bond or a linear alkylene consisting of 1 to 6 carbons, or forming a heterocycle with N;
R₃ consists of a linear or branched alkylene consisting of 1 to 6 carbons; and
R₄ and R₅ are the same or different, each consisting of a hydrogen or a linear or branched alkyl consisting of 1 to 6 carbons;
or a pharmaceutically acceptable salt thereof.

The lipid formulation of may contain one or more ionizable cationic lipids selected from among the following (also referred to herein as “ATX lipids”):

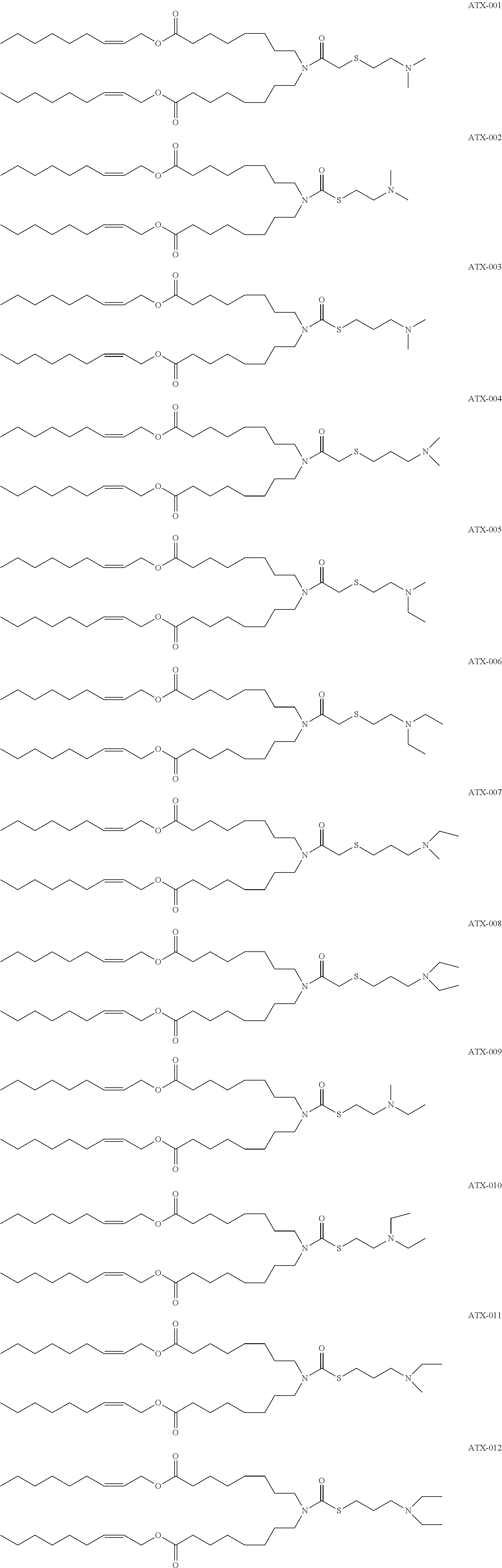

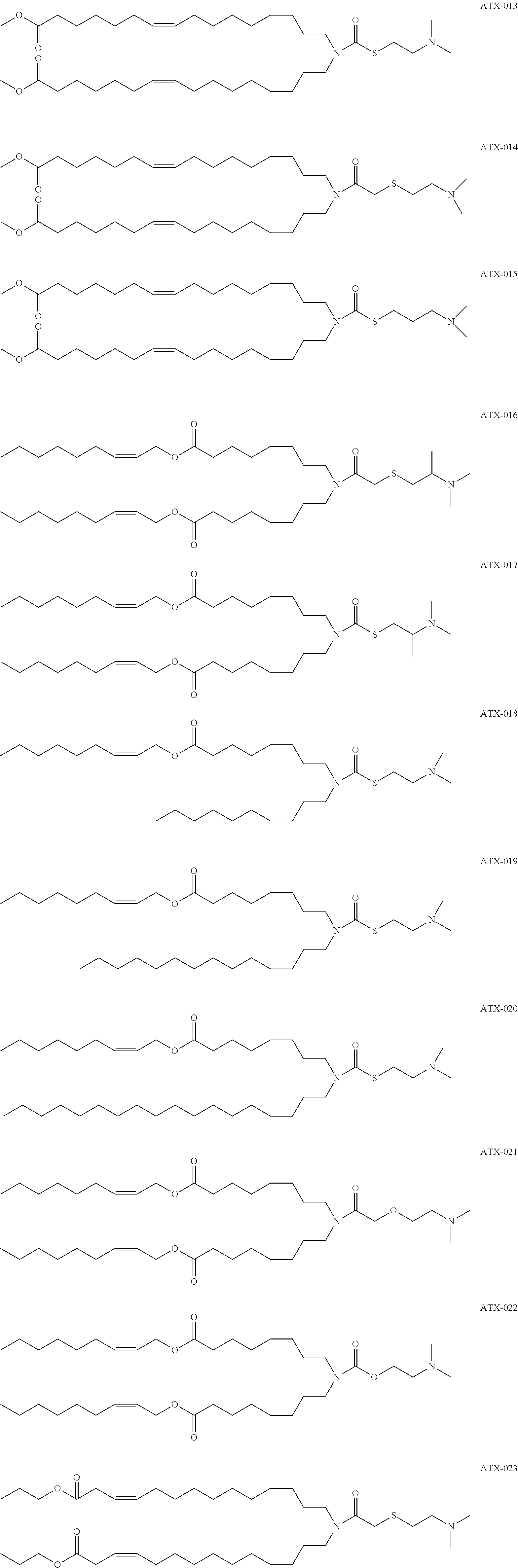

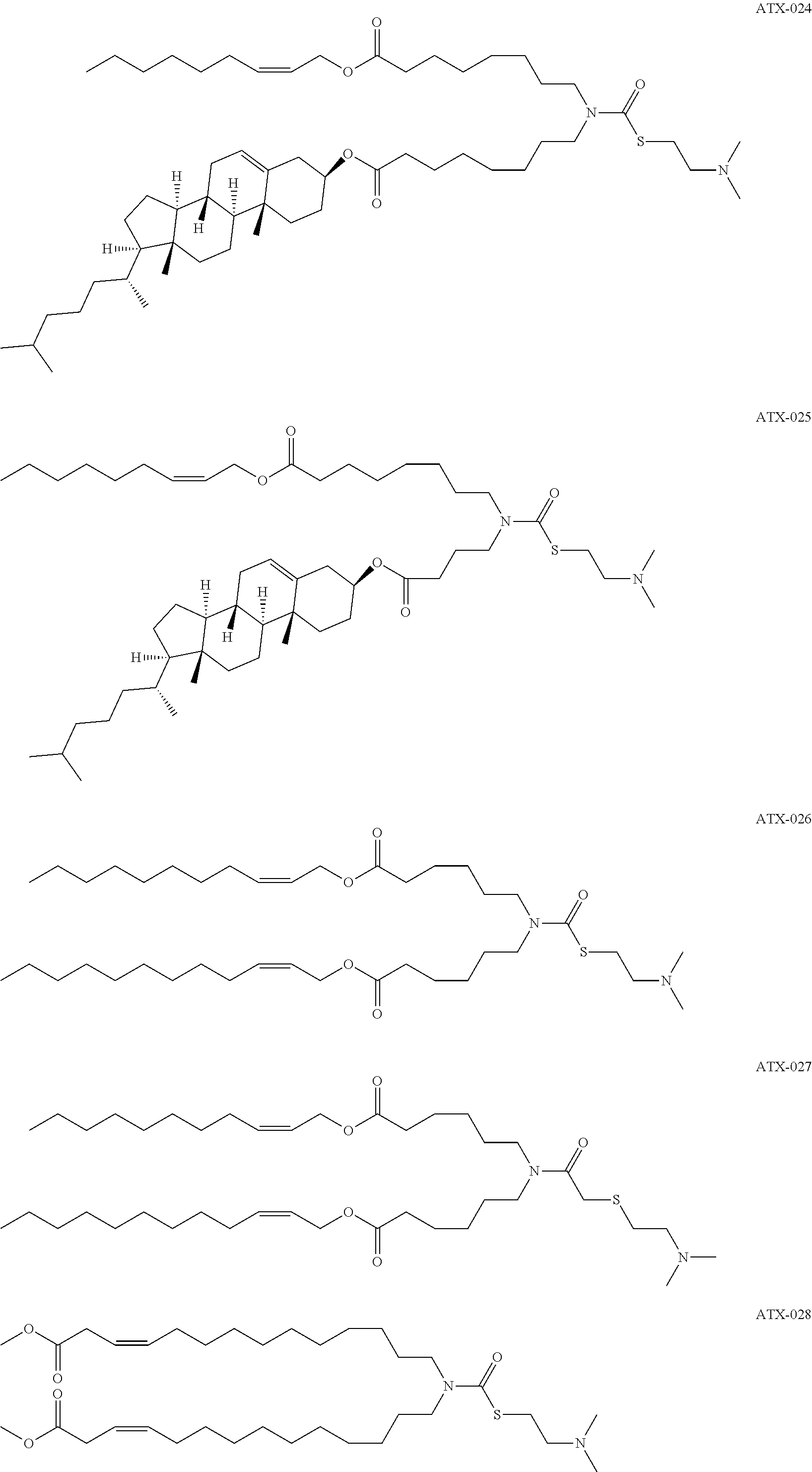

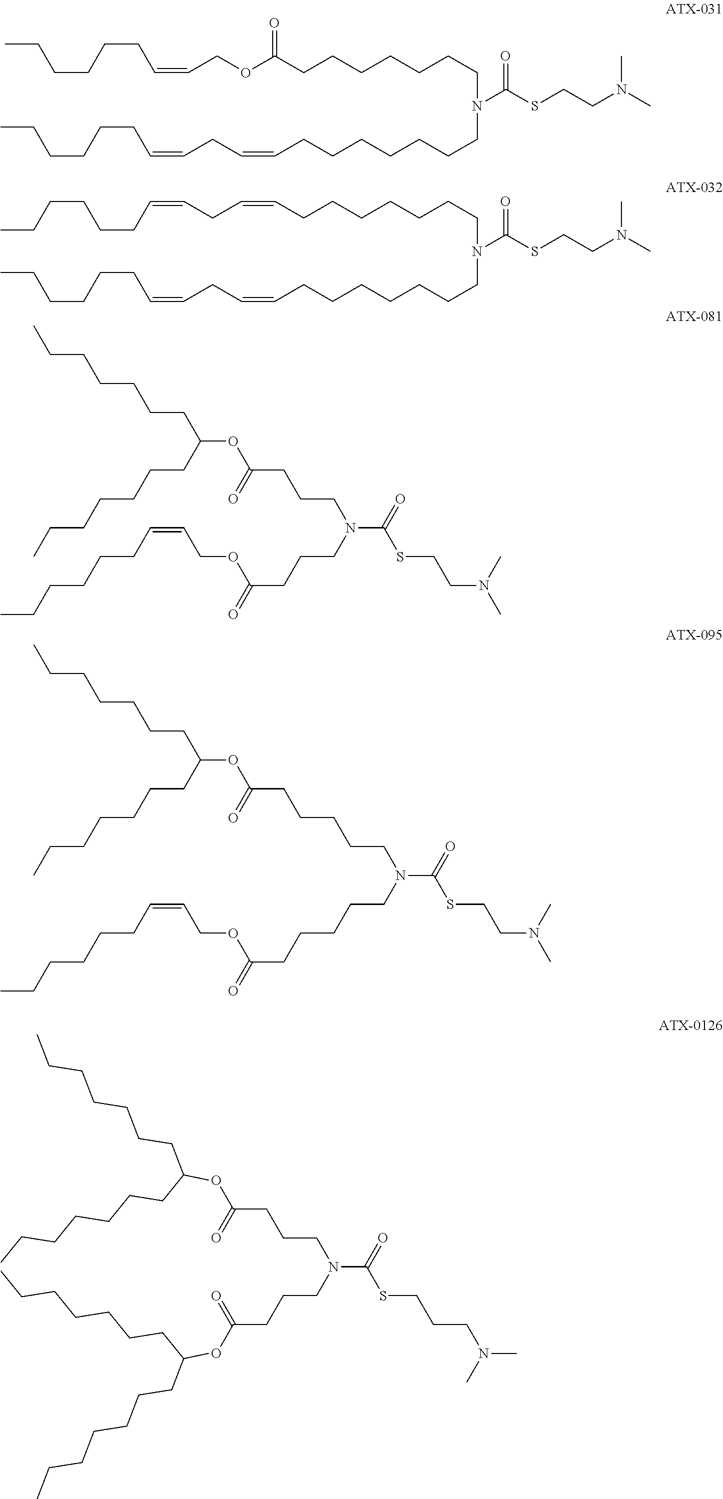

The lipid nanoparticle preferably includes a cationic lipid suitable for forming a lipid nanoparticle. Preferably, the cationic lipid carries a net positive charge at about physiological pH. The cationic lipid may be, for example, N,N-dioleyl-N,N-dimethylammonium chloride (DODAC), N,N-distearyl-N,N-dimethylammonium bromide (DDAB), 1,2-dioleoyltrimethylammoniumpropane chloride (DOTAP) (also known as N-(2,3-dioleoyloxy)propyl)-N,N,N-trimethylammonium chloride and 1,2-Dioleyloxy-3-trimethylaminopropane chloride salt), N-(1-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-di-y-linolenyloxy-N,N-dimethylaminopropane (γ-DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.CI), 1,2-Dilinoleoyl-3-trimethylaminopropane chloride salt (DLin-TAP.CI), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DM A), 2,2-Dilinoleyl-4-dimethylaminomethyl-[1,3]-dioxolane (DLin-K-DMA) or analogs thereof, (3aR,5s,6aS)—N,N-dimethyl-2,2-di((9Z,12Z)-octadeca-9,12-dienyl)tetrahydro-3aH-cyclopenta[d][1,3]dioxol-5-amine, (6Z,9Z,28Z,31Z)-heptatriaconta-6,9,28,31-tetraen-19-yl4-(dimethylamino)butanoate (MC3), 1,1′-(2-(4-(2-((2-(bis(2-hydroxydodecyl)amino)ethyl)(2-hydroxydodecyl)amino)ethyl)piperazin-1-yl)ethylazanediyl)didodecan-2-ol (C12-200), 2,2-dilinoleyl-4-(2-dimethylaminoethyl)-[1,3]-dioxolane (DLin-K-C2-DMA), 2,2-dilinoleyl-4-dimethylaminomethyl-[1,3]-dioxolane (DLin-K-DMA), (6Z,9Z,28Z,31Z)-heptatriaconta-6,9,28 31-tetraen-19-yl 4-(dimethylamino) butanoate (DLin-M-C3-DMA), 3-((6Z,9Z,28Z,31Z)-heptatriaconta-6,9,28,31-tetraen-19-yloxy)-N,N-dimethylpropan-1-amine (MC3 Ether), 4-((6Z,9Z,28Z,31 Z)-heptatriaconta-6,9,28,31-tetraen-19-yloxy)-N,N-dimethylbutan-1-amine (MC4 Ether), or any combination of any of the foregoing. Other cationic lipids include, but are not limited to, N,N-distearyl-N,N-dimethylammonium bromide (DDAB), 3P—(N—(N′,N′-dimethylaminoethane)-carbamoyl)cholesterol (DC-Choi), N-(1-(2,3-dioleyloxy)propyl)-N-2-(sperminecarboxamido)ethyl)-N,N-dimethylammonium trifluoracetate (DOSPA), dioctadecylamidoglycyl carboxyspermine (DOGS), 1,2-dileoyl-sn-3-phosphoethanolamine (DOPE), 1,2-dioleoyl-3-dimethylammonium propane (DODAP), N-(1,2-dimyristyloxyprop-3-yl)-N,N-dimethyl-N-hydroxyethyl ammonium bromide (DMRIE), and 2,2-Dilinoleyl-4-dimethylaminoethyl-[1,3]-dioxolane (XTC). Additionally, commercial preparations of cationic lipids can be used, such as, e.g., LIPOFECTIN (including DOTMA and DOPE, available from GIBCO/BRL), and Lipofectamine (comprising DOSPA and DOPE, available from GIBCO/BRL).

Other suitable cationic lipids are disclosed in International Publication Nos. WO 09/086558, WO 09/127060, WO 10/048536, WO 10/054406, WO 10/088537, WO 10/129709, and WO 2011/153493; U.S. Patent Publication Nos. 2011/0256175, 2012/0128760, and 2012/0027803; U.S. Pat. No. 8,158,601; and Love et al., PNAS, 107(5), 1864-69, 2010.

Other suitable amino lipids include those having alternative fatty acid groups and other dialkylamino groups, including those, in which the alkyl substituents are different (e.g., N-ethyl-N-methylamino-, and N-propyl-N-ethylamino-). In general, amino lipids having less saturated acyl chains are more easily sized, particularly when the complexes must be sized below about 0.3 microns, for purposes of filter sterilization. Amino lipids containing unsaturated fatty acids with carbon chain lengths in the range of C14 to C22 may be used. Other scaffolds can also be used to separate the amino group and the fatty acid or fatty alkyl portion of the amino lipid.

Preferably, the LNP comprises the cationic lipid with formula (III) according to the patent application PCT/EP2017/064066. In this context, the disclosure of PCT/EP2017/064066 is also incorporated herein by reference.

Preferably, amino or cationic lipids of the disclosure have at least one protonatable or deprotonatable group, such that the lipid is positively charged at a pH at or below physiological pH (e.g. pH 7.4), and neutral at a second pH, preferably at or above physiological pH. It will, of course, be understood that the addition or removal of protons as a function of pH is an equilibrium process, and that the reference to a charged or a neutral lipid refers to the nature of the predominant species and does not require that all of the lipid be present in the charged or neutral form. Lipids that have more than one protonatable or deprotonatable group, or which are zwitterionic, are not excluded from use in the disclosure. In certain embodiments, the protonatable lipids have a pKa of the protonatable group in the range of about 4 to about 11, e.g., a pKa of about 5 to about 7.

The cationic lipid can comprise from about 20 mol % to about 70 or 75 mol % or from about 45 to about 65 mol % or about 20, 25, 30, 35, 40, 45, 50, 55, 60, 65, or about 70 mol % of the total lipid present in the particle. In another embodiment, the lipid nanoparticles include from about 25% to about 75% on a molar basis of cationic lipid, e.g., from about 20 to about 70%, from about 35 to about 65%, from about 45 to about 65%, about 60%, about 57.5%, about 57.1%, about 50% or about 40% on a molar basis (based upon 100% total moles of lipid in the lipid nanoparticle). In one embodiment, the ratio of cationic lipid to nucleic acid is from about 3 to about 15, such as from about 5 to about 13 or from about 7 to about 11.

### Pharmaceutical Compositions

Preferably, the disclosure provides pharmaceutical compositions containing a codon optimized mRNA encoding a human OTC protein of SEQ ID NO: 3 or SEQ ID NO: 4, preferably formulated in a lipid delivery system or lipid carrier and preferably comprising pharmaceutically acceptable excipients. Pharmaceutical compositions disclosed herein preferably facilitate expression of mRNA in vivo.

Suitable routes of administration include, for example, oral, rectal, vaginal, transmucosal, pulmonary including intratracheal or inhaled, or intestinal administration; parenteral delivery, including intradermal, transdermal (topical), intramuscular, subcutaneous, intramedullary injections, as well as intrathecal, direct intraventricular, intravenous, intraperitoneal, or intranasal.

Preferably, the intramuscular administration is to a muscle selected from the group consisting of skeletal muscle, smooth muscle and cardiac muscle. In some embodiments the administration results in delivery of the mRNA to a muscle cell. In some embodiments the administration results in delivery of the mRNA to a hepatocyte (i.e., liver cell). In a particular embodiment, the intramuscular administration results in delivery of the mRNA to a muscle cell.

Preferably, mRNAs and lipid formulations thereof may be administered in a local rather than systemic manner, for example, via injection of the pharmaceutical composition directly into a targeted tissue, preferably in a sustained release formulation. Local delivery can be affected in various ways, depending on the tissue to be targeted. For example, aerosols containing compositions of the present disclosure can be inhaled (for nasal, tracheal, or bronchial delivery); can be supplied in liquid, tablet or capsule form for administration to the stomach or intestines, can be supplied in suppository form for rectal or vaginal application; or can even be delivered to the eye by use of creams, drops, or even injection. Formulations containing provided compositions complexed with therapeutic molecules or ligands can even be surgically administered, for example in association with a polymer or other structure or substance that can allow the compositions to diffuse from the site of implantation to surrounding cells. Alternatively, they can be applied surgically without the use of polymers or supports.

Pharmaceutical compositions may be administered to any desired tissue. In some embodiments, the OTC mRNA delivered by provided liposomes or compositions is expressed in the tissue in which the liposomes and/or compositions were administered. In some embodiments, the mRNA delivered is expressed in a tissue different from the tissue in which the liposomes and/or compositions were administered. Exemplary tissues in which delivered mRNA may be delivered and/or expressed include, but are not limited to the liver, kidney, heart, spleen, serum, brain, skeletal muscle, lymph nodes, skin, and/or cerebrospinal fluid.

Preferably, a pharmaceutical composition can contain a polynucleotide described herein such as a primary DNA construct or mRNA described herein within a viral or bacterial vector.

Preferably, the primary DNA construct for an mRNA described herein or an mRNA described herein can be formulated using one or more excipients to: (1) increase stability; (2) increase cell transfection; (3) permit a sustained or delayed release (e.g., from a depot formulation of the polynucleotide, primary construct, or mRNA); (4) alter the biodistribution (e.g., target the polynucleotide, primary construct, or mRNA to specific tissues or cell types); (5) increase the translation of encoded protein in vivo; and/or (6) alter the release profile of encoded protein in vivo.

In addition to traditional excipients such as any and all solvents, dispersion media, diluents, or other liquid vehicles, dispersion or suspension aids, surface active agents, isotonic agents, thickening or emulsifying agents, preservatives, excipients of the present disclosure can include, without limitation, liposomes, lipid nanoparticles, polymers, lipoplexes, core-shell nanoparticles, peptides, proteins, cells transfected with primary DNA construct, or mRNA (e.g., for transplantation into a subject), hyaluronidase, nanoparticle mimics and combinations thereof.

Accordingly, the formulations described herein can include one or more excipients, each in an amount that together increases the stability of the primary DNA construct, or mRNA, increases cell transfection by the primary construct, or mRNA, increases the expression of polynucleotide, primary construct, or mRNA encoded protein, and/or alters the release profile of polynucleotide, primary construct, or mRNA encoded proteins. Further, the primary construct and mRNA of the present disclosure may be formulated using self-assembled nucleic acid nanoparticles.

Formulations of the pharmaceutical compositions described herein may be prepared by any method known or hereafter developed in the art of pharmacology. In general, such preparatory methods include the step of associating the active ingredient with an excipient and/or one or more other accessory ingredients.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient may generally be equal to the dosage of the active ingredient which would be administered to a subject and/or a convenient fraction of such a dosage including, but not limited to, one-half or one-third of such a dosage.

Relative amounts of the active ingredient, the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient.

Pharmaceutical formulations may additionally comprise a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired.

Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; incorporated herein by reference in its entirety). The use of a conventional excipient medium may be contemplated within the scope of the embodiments of the present disclosure, except insofar as any conventional excipient medium may be incompatible with a substance or its derivatives, such as by producing any undesirable biological effect or otherwise interacting in a deleterious manner with any other component(s) of the pharmaceutical composition.

### Therapeutic Uses

The mRNA sequences, primary DNA constructs that transcribe the mRNA sequences described herein and pharmaceutical compositions thereof provide numerous in vivo and in vitro methods and are useful to treat OTC deficiency. The treatment may comprise treating a human patient with OTC deficiency. Similarly, compositions described herein, may be used in vitro or ex vivo to study OTC deficiency in cell or animal-based models. For example, cells deficient for OTC expression can be used to analyze the ability to restore OTC expression and/or activity, as well as the time period over which expression and/or activity persists. Such cells and animal models are also suitable to identify other factors involved in the pathway, whether binding partners or factors in the same biochemical pathway. In other embodiments, compositions described herein can be used to study or track mitochondrial delivery.

Polynucleotides described herein, such as a DNA construct or template or an mRNA sequence described herein can be delivered to patients or cells experiencing OTC deficiency. Preferably the mRNA sequence comprises SEQ ID NO: 119 encoding a modified OTC protein of SEQ ID NO: 4. Preferably the DNA sequence comprises SEQ ID NO: 221 encoding a modified OTC protein of SEQ ID NO: 4.

Following administration, OTC is expressed in the cells or subject. Preferably, compositions described herein are delivered to mitochondria. Preferably compositions described herein are delivered to liver cells.

Preferably the therapeutic methods described herein decrease ammonia levels in plasma and/or urine in a subject in need thereof or in cells in culture, such as a subject having an OTC deficiency. Preferably, the therapeutic methods described herein decrease orotic acid levels in plasma and/or urine in a subject in need thereof or in cells in culture. Preferably, the therapeutic methods described herein increase citrulline in plasma and/or urine in a subject in need thereof or in cells in culture. Preferably, ammonia levels, orotic acid levels and/or citrulline levels are used as biomarkers to (i) identify subjects in need of treatment and/or (ii) to evaluate efficacy of treatment using the mRNA or DNA templates described herein.

Examples of mRNA sequences for use with these methods include those listed in Table 5. Preferably cDNA templates used to transcribe the mRNA sequences described herein are listed in Table 6. Preferred mRNA sequences for administering to patients for treatment of OTC deficiency are SEQ ID NOS: 1799 and SEQ ID NOS: 1921. Preferably the preferred mRNA sequences of SEQ ID NO: 1799 and SEQ ID NO: 1921.

### Dosing

An effective dose of a mRNA, a protein or pharmaceutical formulations thereof of the present disclosure can be an amount that is sufficient to treat ORF protein deficiency in a cell and/or in a patient. A therapeutically effective dose can be an amount of an agent or formulation that is sufficient to cause a therapeutic effect. A therapeutically effective dose can be administered in one or more separate administrations, and by different routes. Generally, a therapeutically effective amount is sufficient to achieve a meaningful benefit to the subject (e.g., treating, modulating, curing, preventing and/or ameliorating phenylketonuria). For example, a therapeutically effective amount may be an amount sufficient to achieve a desired therapeutic and/or prophylactic effect. Generally, the amount of a therapeutic agent administered to a subject in need thereof will depend upon the characteristics of the subject. Such characteristics include the condition, disease severity, general health, age, sex and body weight of the subject. One of ordinary skill in the art will be readily able to determine appropriate dosages depending on these and other related factors. In addition, both objective and subjective assays may optionally be employed to identify optimal dosage ranges.

Methods provided herein contemplate single as well as multiple administrations of a therapeutically effective amount of an mRNA sequence described herein. Pharmaceutical compositions comprising an mRNA sequence encoding an ORF protein described herein can be administered at regular intervals, depending on the nature, severity and extent of the subject's condition. Preferably, a therapeutically effective amount an mRNA sequence of the present disclosure may be administered periodically at regular intervals (e.g., once every year, once every six months, once every four months, once every three months, once every two months, once a month), biweekly, weekly, daily, twice a day, three times a day, four times a day, five times a day, six times a day, or continuously.

Preferably, the pharmaceutical compositions of the mRNA of the present disclosure are formulated such that they are suitable for extended-release of the translatable compound encoding a modified protein described herein contained therein. Such extended-release compositions may be conveniently administered to a subject at extended dosing intervals. For instance, in one embodiment, the pharmaceutical compositions of the present disclosure are administered to a subject twice a day, daily or every other day. In some embodiments, the pharmaceutical compositions of the present disclosure are administered to a subject twice a week, once a week, every 10 days, every two weeks, every 28 days, every month, every six weeks, every eight weeks, every other month, every three months, every four months, every six months, every nine months or once a year. Also contemplated herein are pharmaceutical compositions which are formulated for depot administration (e.g., subcutaneously, intramuscularly) to either deliver or release an mRNA sequence encoding an OTC protein described herein over extended periods of time. Preferably, the extended-release means employed are combined with modifications made to the translatable compound encoding an OTC protein described herein to enhance stability.

A therapeutically effective dose, upon administration, can result in serum or plasma levels of OTC of 1-1000 pg/ml, or 1-1000 ng/ml, or 1-1000 μg/ml, or more. In some embodiments, administering a therapeutically effective dose of a composition comprising an mRNA sequence described herein can result in increased liver modified protein levels in a treated subject. Preferably, administering a composition comprising a mRNA described herein results in a 5%, 10%, 20%, 30%, 40%, 50%, 60%, 70%, 80%, 90%, or 95% increase in liver modified protein levels relative to a baseline modified protein level in the subject prior to treatment. Preferably, administering a therapeutically effective dose of a composition comprising an mRNA described herein will result an increase in liver OTC levels relative to baseline liver OTC levels in the subject prior to treatment. In some embodiments, the increase in liver OTC levels relative to baseline liver OTC levels will be at least 5%, 10%, 20%, 30%, 40%, 50%, 100%, 200%, or more.

Preferably, a therapeutically effective dose, when administered regularly, results in increased expression of OTC in the liver as compared to baseline levels prior to treatment. Preferably, administering a therapeutically effective dose of a composition comprising an mRNA sequence described herein results in the expression of a modified protein level at or above about 10 ng/mg, about 20 ng/mg, about 50 ng/mg, about 100 ng/mg, about 150 ng/mg, about 200 ng/mg, about 250 ng/mg, about 300 ng/mg, about 350 ng/mg, about 400 ng/mg, about 450 ng/mg, about 500 ng/mg, about 600 ng/mg, about 700 ng/mg, about 800 ng/mg, about 900 ng/mg, about 1000 ng/mg, about 1200 ng/mg or about 1500 ng/mg of the total protein in the liver of a treated subject.

Preferably, a therapeutically effective dose, when administered regularly, results in a reduction of orotic acid levels in a biological sample. In some embodiments, administering a therapeutically effective dose of a composition comprising an mRNA described herein results in a reduction of orotic acid levels in a biological sample (e.g., urine, plasma or serum sample) by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% as compared to baseline orotic acid levels before treatment. Preferably, the biological sample is selected from plasma, serum, whole blood, urine, or cerebrospinal fluid. Preferably, administering a therapeutically effective dose of a composition comprising an mRNA described herein results in reduction of orotic acid levels to about 1000 μmol/L or less, about 900 μmol/L or less, about 800 μmol/L or less, about μmol/L or less, about 600 μmol/L or less, about 500 μmol/L or less, about 400 μmol/L or less, about 300 μmol/L or less, about 200 μmol/L or less, about 100 μmol/L or less or about 50 μmol/L or less in serum or plasma. Preferably, a therapeutically effective dose, when administered regularly results in reduction of orotic acid levels to about 600 μmol/L or less in serum or plasma. In another exemplary embodiment, a therapeutically effective dose, when administered regularly results in reduction of orotic acid levels to about 360 μmol/L or less in serum or plasma. Preferably, a therapeutically effective dose, when administered regularly results in reduction of orotic acid levels to about 120 μmol/L or less in serum or plasma.

A therapeutically effective dose of an mRNA described herein in vivo can be a dose of about 0.001 to about 500 mg/kg body weight. For instance, the therapeutically effective dose may be about 0.001-0.01 mg/kg body weight, or 0.01-0.1 mg/kg, or 0.1-1 mg/kg, or 1-10 mg/kg, or 10-100 mg/kg. Preferably, a Lipid-enabled and Unlocked Nucleomonomer Agent modified RNA (LUNAR)-mRNA (see WO 2015/074085 and U.S. 2018/0169268), encoding an OTC protein described herein, is provided at a dose ranging from about 0.1 to about 10 mg/kg body weight.

### Combinations

The cDNA primary constructs, mRNA or encoded OTC proteins described herein may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent. Preferably, the methods of treatment of the present disclosure encompass the delivery of pharmaceutical, prophylactic, diagnostic, or imaging compositions in combination with agents that may improve their bioavailability, reduce and/or modify their metabolism, inhibit their excretion, and/or modify their distribution within the body. As a non-limiting example, mRNA disclosed herein and preferably an mRNA sequence comprising SEQ ID NO: 119, encoding a modified OTC protein of SEQ ID NO: 4 may be used in combination with a pharmaceutical agent for the treatment of OTC deficiency. The pharmaceutical agent includes, but is not limited to one or more of sodium phenylbutyrate, glycerol phenylbutyrate, sodium phenylacetate, sodium benzoate, arginine, citrulline, Multiple vitamins, calcium supplements or combined with low protein/high caloric diet. In general, it is expected that agents utilized in combination with be utilized at levels that do not exceed the levels at which they are utilized individually. In some embodiments, the levels utilized in combination will be lower than those utilized individually. In one embodiment, the combinations, each or together may be administered according to the split dosing regimens as are known in the art.

### EXAMPLES

### Example 1: Material and Methods

### In Vitro Transcription Protocol

The mRNAs were synthesized in vitro using T7RNA polymerase-mediated DNA-dependent RNA transcription where uridine triphosphate (UTP) was substituted and unsubstituted with modified UTPs such as 5 methoxy UTP (5MeOU), N1-methoxy methyl pseudo UTP (N1-MOM), 5-hydroxy methyl UTP, 5-carboxy UTP, and mixture of modifications using linearized template for each UTR combination. The mRNA was purified using column chromatography, the DNA and double stranded mRNA contamination of all mRNAs was removed using an enzymatic reaction, and the mRNA was concentrated, and buffer exchanged.

Preparation of Lipid Encapsulated mRNA

Lipid encapsulated mRNA particles were prepared by mixing lipids (ATX lipid:DSPC:Cholesterol:PEG-DMG) in ethanol with OTC mRNA dissolved in Citrate buffer. The mixed material was instantaneously diluted with Phosphate Buffer. Ethanol was removed by dialysis against phosphate buffer using regenerated cellulose membrane (100 kD MWCO) or by tangential flow filtration (TFF) using modified polyethersulfone (mPES) hollow fiber membranes (100 kD MWCO). Once the ethanol was completely removed, the buffer was exchanged with HEPES (4-(2-hydroxyethyl)-1-piperazineethanesulfonic acid) buffer containing 50 mM NaCl and 9% sucrose, pH 7.3. The formulation was concentrated followed by 0.2 μm filtration using PES filters. The mRNA concentration in the formulation was then measured by Ribogreen fluorimetric assay following which the concentration was adjusted to a final desired concentration by diluting with HEPES buffer containing 50 mM NaCl, 9% sucrose, pH 7.3 containing glycerol. The final formulation was then filtered through a 0.2 μm filter and filled into glass vials, stoppered, capped and placed at −70±5° C. The frozen formulations were characterized for their mRNA content and percent encapsulation by Ribogreen assay, mRNA integrity by fragment analyzer, lipid content by high performance liquid chromatography (HPLC), particle size by dynamic light scattering on a Malvern Zetasizer Nano ZS, pH and Osmolality.

### In-Cell Western (ICW)

96-well collagen plates were used to seed the cells at the appropriate density in Dulbecco's Modified Eagle Media (DMEM)/Fetal Bovine Serum (FBS) culture media. At the optimal confluence, cells were transfected with the targeted mRNAs diluted in the transfection reagent mix (MessengerMax and Opti-MEM). Cells were placed in the CO₂ incubator and allowed to grow. At the desire timepoint, media was removed, and cells were fixed in 4% fresh paraformaldehyde (PFA) for 20 min. After that, fixative was removed, and cells were permeabilize in tris buffered saline with TWEEN (TBST) for 5 min several times. When permeabilization washes were complete, cells were incubated with a blocking buffer (ODYSSEY® Blocking Buffer (PBS) (Li-Cor, Lincoln, NE)) for 45 min. Primary antibody was then added and incubated for 1 h at room temperature. Cells were then washed several times in TBST and incubated for 1 h with a secondary antibody diluted in blocking buffer and containing a CellTag 700 stain. To finalize, cells were washed several times in TBST followed by a last wash in tris-buffered saline (TBS). The plate was imaged using the Licor detection system and data was normalized to the total number of cells labeled by the CellTag 700.

### Example 2: UTRs Screening in Hepa1,6 and Hep3B—Correlation at 24 h and 48 h

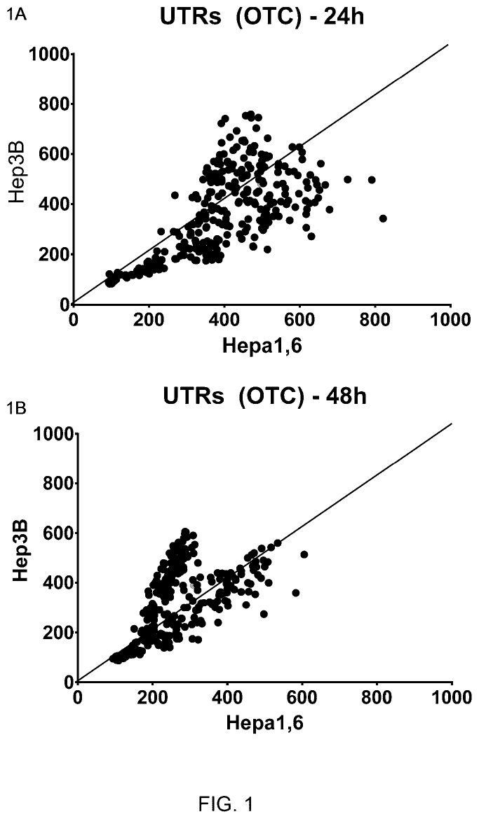

A UTR library was screened in vitro using mRNA construct #571 comprising the sequence of SEQ ID NO: 34 as CDS (coding sequence). In-Cell Western assays as described in Example 1 were used to transfect the different mRNAs into Hepa1,6 and Hep3B using commercially available transfection reagents. OTC protein expression levels were measured by near-infrared fluorescent imaging systems. Commercially available antibodies for OTC were used for detection. Untransfected and reference sequences were used as internal controls. FIG. 1, Panel A is a scatter plot of OTC protein expression levels found in Hepa1,6 and Hep3B cells at 24 hours. FIG. 1, Panel B is a scatter plot of OTC protein expression levels found in Hepa1,6 and Hep3B cells at 48 hours. The aim of the screening was to determine an UTR-specific impact on OTC expression levels in a human (Hep3B) and a mouse (Hepa1,6) liver cell line that will be beneficial to determine which UTRs would work best in both models, in particular, its translatability from mouse-to-human. Top expressing UTRs were used in further profiling studies.

### Example 3: Round 1 of Protein Stability Compounds Screened in Hepa1,6 and Hep3B at 24 h—Correlation

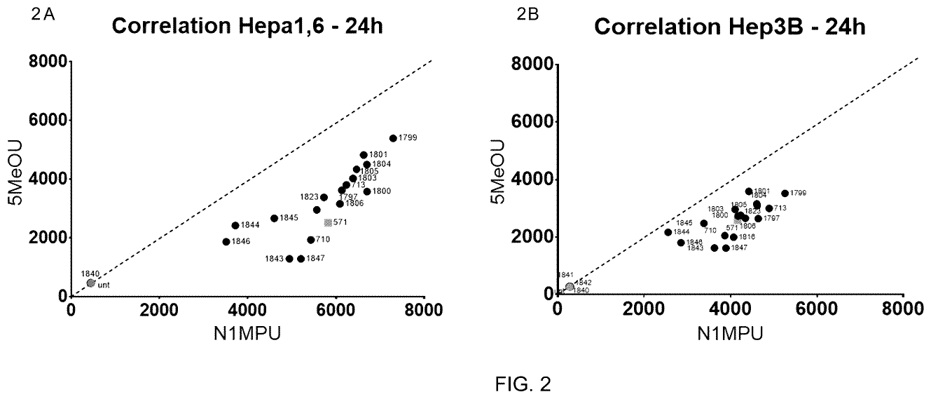

In vitro screening of certain mRNA constructs of Table 5 that were designed based on a protein-stability approach was performed. The mRNA constructs were tested in two different chemistries N¹-methylpseudouridine (N1MPU) and 5-methoxyuridine (5MeOU) meaning that 100% of the uridines in each mRNA were N1MPU only or 5MeOU only (not a combination of 5MeOU or N1MPU). In-Cell Western (ICW) assays as described in Example 1 were used to transfect the different mRNAs into Hepa1,6 and Hep3B using commercially available transfection reagents. OTC protein expression levels were measured by near-infrared fluorescent imaging systems. Commercially available antibodies for OTC were used for detection. Untransfected and reference sequences were used as internal controls. FIG. 2, panel A is a scatter plot showing the correlation of OTC protein expression levels in Hepa1,6 cells at 24 hours as a function of mRNAs tested in N1MPU and 5MeOU chemistries. FIG. 2, panel B is a scatter plot showing the correlation of OTC protein expression levels in Hep3B cells at 24 hours as a function of mRNAs tested in N1MPU and 5MeOU chemistries. These figures exhibit the degree of variability in expression levels when mRNAs from two different chemistries are tested in a mouse and a human liver cell line. It can be seen that that in this experiment, most of the compounds express better when an N1MPU chemistry is used in the mRNA.

### Example 4: Round 2 of Protein Stability Compounds Screened in Human Primary Hepatocytes at 24 h and 48 h—Correlation

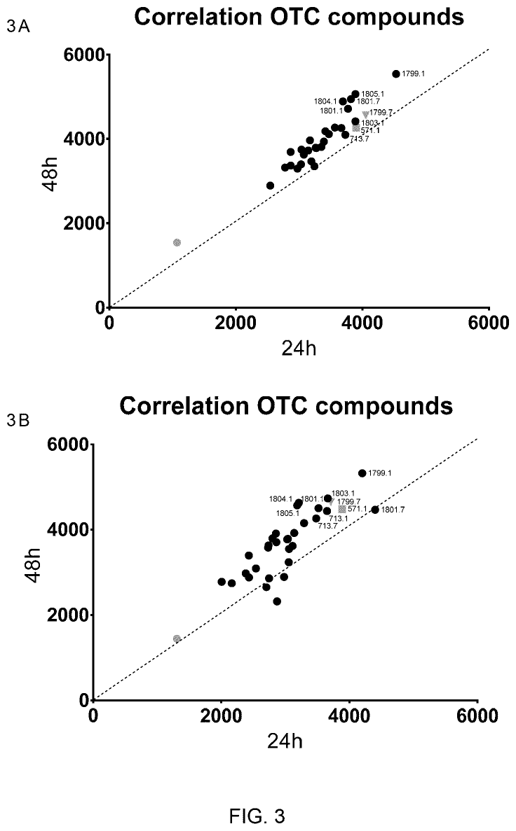

In vitro screening of certain mRNA constructs of Table 5 that were designed based on a protein stability approach was performed. The mRNAs were tested in two different chemistries, 100% of the Uridines are N1MPU indicated by the name of mRNA constructs followed by “0.1” and 100% of the uridines are 5MeOU indicated by the name of mRNA constructs followed by “0.7”. In-Cell Western (ICW) assays as described in Example 1 were used to transfect the different mRNAs into human primary hepatocytes using commercially available transfection reagents. OTC protein expression levels were measured by near-infrared fluorescent imaging systems. Commercially available antibodies for OTC were used for detection. Untransfected and reference sequences were used as internal controls. (FIG. 3, Panels A and B). The results indicate that, in contrast to the experiments conducted in cancer cell lines (Hepa1,6; Hep3B; Example 3, both chemistries express similarly in human primary hepatocytes.

### Example 5: Round 3 of Protein Stability Compounds Screening in Human Primary Hepatocytes at 24 h and 48 h—Correlation

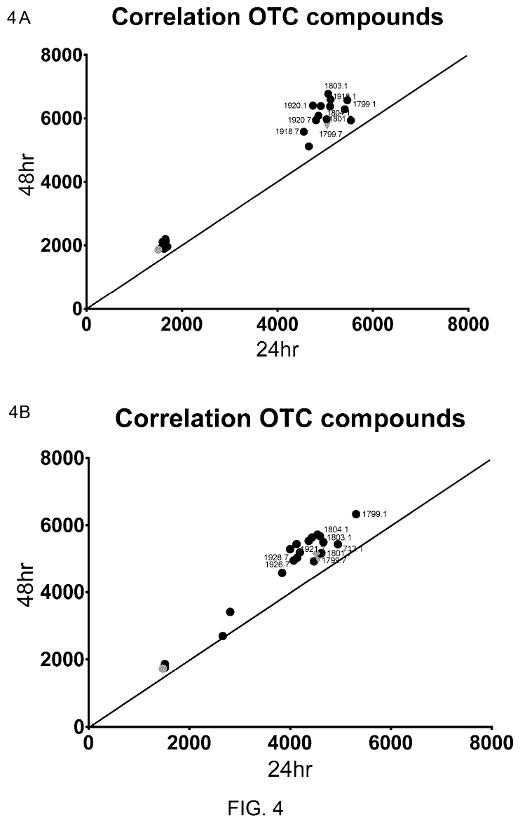

In vitro screening of novel compounds designed based on a protein stability approach was performed. mRNAs were tested in two different chemistries, N1MPU indicated by the name of mRNA constructs followed by “0.1” and 5MeOU indicated by the name of mRNA constructs followed by “0.7”. In-Cell Western (ICW) assays as described in Example 1 were used to transfect the different mRNAs into human primary hepatocytes using commercially-available transfection reagents. OTC protein expression levels were measured by near-infrared fluorescent imaging systems. Commercially available antibodies for OTC protein were used for detection. Untransfected and reference sequences were used as internal controls. (FIG. 4, Panels A and B). The results indicate that, in contrast to the experiments conducted in cancer cell lines (Hepa1,6; Hep3B; Example 3), both chemistries express similarly in human primary hepatocyte.

### Example 6: OTC Protein-Expression Levels in Human Primary Cells Transfected with OTC mRNA Constructs 1799.7 (5MeOU Chemistry) Encoding the OTC Protein of SEQ ID NO: 3 and 1921.7 (5MeOU Chemistry) Encoding the Modified OTC Protein of SEQ ID NO: 4

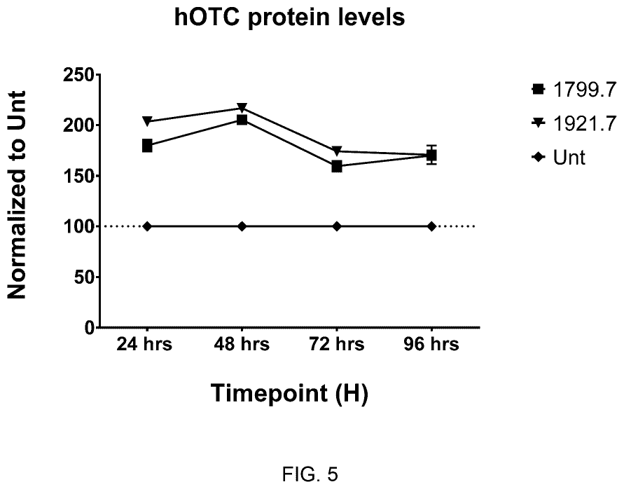

In-Cell Western (ICW) assays as described in Example 1 were used to transfect OTC mRNAs into human primary hepatocytes using commercially available transfection reagents. OTC protein expression levels were measured by near-infrared fluorescent imaging systems during a time course study up to 96 h. Commercially available OTC antibodies were used for detection. Untransfected cells were used as internal control. Plot shows OTC protein levels normalized to untransfected controls. (FIG. 5). The purpose of this study was to evaluate the half-life of the unmodified versus the modified protein sequence (encoded by constructs 1799.7, 1921.7, respectively) under in vitro conditions in transfected human primary hepatocytes. The results indicate that 1921.7 demonstrated more stable expression than 1799.7

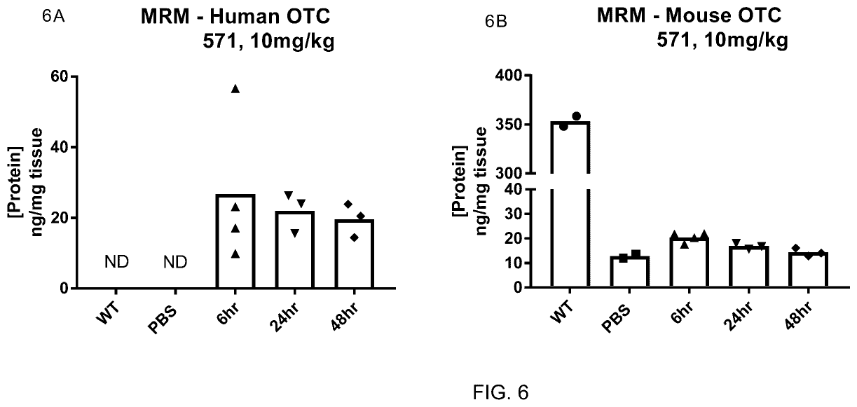

Example 7: OTC expression levels measured by multiple reaction monitoring (MRM) mass spectrometry in spf/ash mice dosed at 10 mg/kg. Spf/ash mice received an IV injection with either PBS or lipid-formulated (as described in Example 1) OTC-mRNAs at a 10 mg/kg dosing. WT mice were used as internal controls to determine endogenous levels. A time course (6 h, 24 h and 48 h) was performed, and expression levels were measured by MRM using human and mouse specific epitopes for OTC. Graphs were made that represent the amount of protein (ng/mg tissue) detected by MRM specific for human OTC (FIG. 6, Panel A) or mouse (FIG. 6, Panel B). Human- and mouse-specific heavy peptides were designed to measure total levels of OTC in both species. This data set shows quantitative levels of human-specific OTC derived from translation of delivered mRNAs are detected. This expression maintains a high level of stability up to 48 h.

### Example 8: OTC Expression Levels Measured by Western Blot in WT Mice Dosed at 3 mg/kg

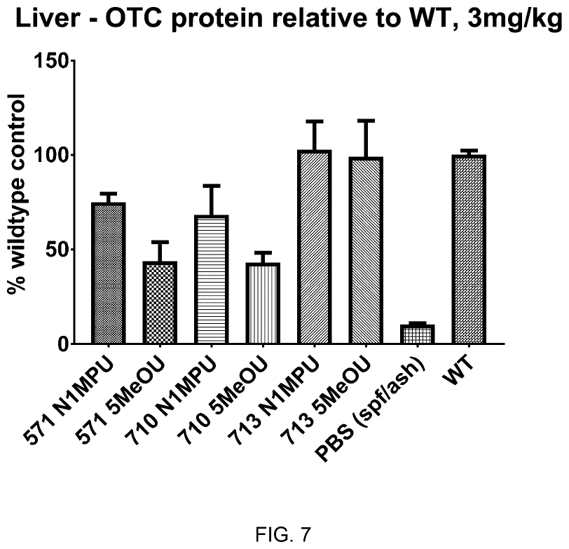

Spf/ash mice received an IV injection with either phosphate buffered saline (PBS) or lipid-formulated OTC-mRNAs at a 3 mg/kg dosing using two different chemistries (N1MPU and 5MeOU). WT mice were used as internal controls to determine endogenous levels. Animals were sacrificed 24 h post-dose. OTC expression levels were measured by Western Blot (WB) using an OTC specific antibody. In the results provided in FIG. 7, the bars represent the percentage of expression relative to WT levels (100%). The data generated in this figure shows that WT levels of total OTC in a mouse background were achieved for several codon-optimized sequences.

### Example 9: OTC Expression Levels Measured by MRM in a Dose Range Finding Study

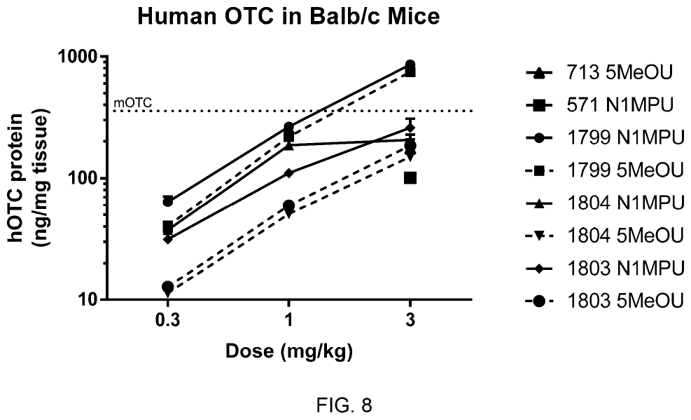

Balb/c mice received an IV injection with either PBS or lipid-formulated OTC-mRNAs at three different doses: 0.3 mg/kg, 1 mg/kg and 3 mg/kg and using two different chemistries (N1MPU and 5MeOU). Animals were sacrificed 24 h post-dose and expression levels were measured by MRM using human and mouse specific epitopes for OTC. The graph in FIG. 8 represents the percentage of expression of human OTC (ng) per mg of liver tissue in Balb/c mice. The horizontal dotted line represents the relative mouse OTC levels in Balb/c mice. (FIG. 8). Expression levels of hOTC protein for mRNA construct 713 5MeOU and mRNA construct 571 N1MPU are indicated by arrows in FIG. 8. In this figure, the MRM was used to quantitatively determine human-selective and mouse-selective OTC protein levels. The data generated in this figure shows, in a dose dependent manner, that WT levels of human OTC in a mouse background are achieved with the codon-optimized sequences disclosed herein.

### Example 10: Urinary Orotate Levels Measured in PBS and Treated Spf/Ash Mice

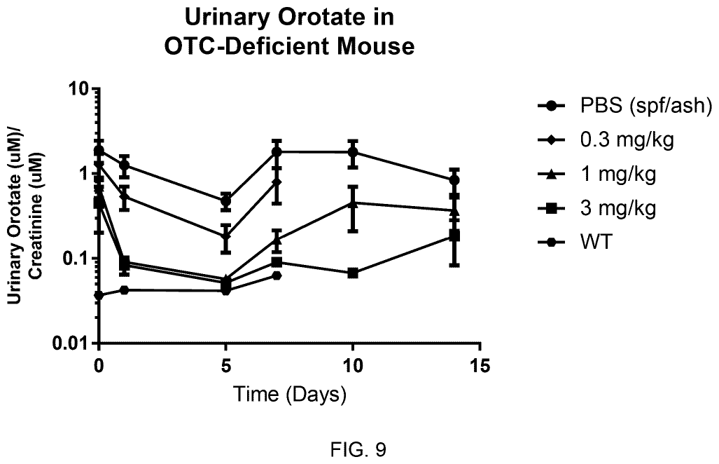

Spf/ash mice received an IV injection with either PBS or lipid-formulated OTC-mRNA construct 1799.7 (5MeOU chemistry) at three different doses: 0.3 mg/kg, 1 mg/kg and 3 mg/kg. WT and spf/ash mice were used to determine baseline and high urinary orotate levels, respectively. A spf/ash time course was determined, and urinary orotate levels were measured at each timepoint. The results can be seen in FIG. 9. Urinary orotate was normalized to creatinine, which is represented in the graph in the Y axis throughout the time course and serve as a proof-of-concept of functional restoration of OTC activity post-injection. At 3 mg/kg, a sustainable reduction of urinary orotate levels up to 14 days was observed.

### Example 11: Pharmacokinetic/Pharmacodynamic (PK/PD) Analysis Comparing Human OTC Expression Levels and Urinary Orotate at 96 h

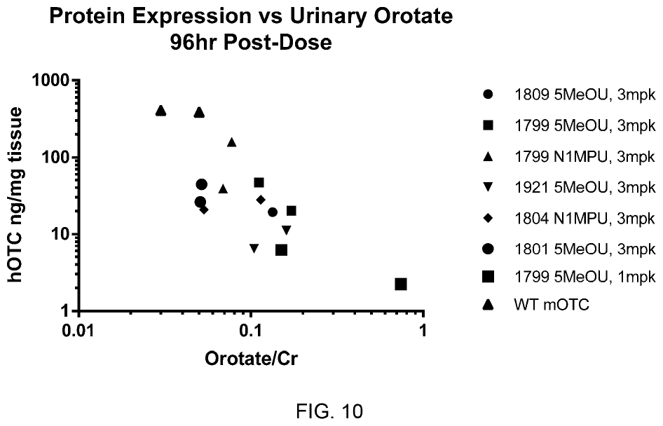

Spf/ash mice received an IV injection with either PBS or certain lipid-formulated OTC-mRNAs from Table 5 at 1 mg/kg and 3 mg/kg using two different chemistries (N1MPU and 5MeOU). WT mice were used as internal controls. Human-specific OTC levels were measured by MRM whereas urinary orotate was determined in each sample and normalized to creatinine. PK/PD is plotted in FIG. 10. PK/PD analysis shows the correlation between protein expression levels and reduction of urinary orotate in a compound-specific manner. Construct 1799.7 shows a high PK/PD correlation.

### Example 12: Fractioning of Spf/Ash Mice In Vivo Samples Treated with Selected mRNAs

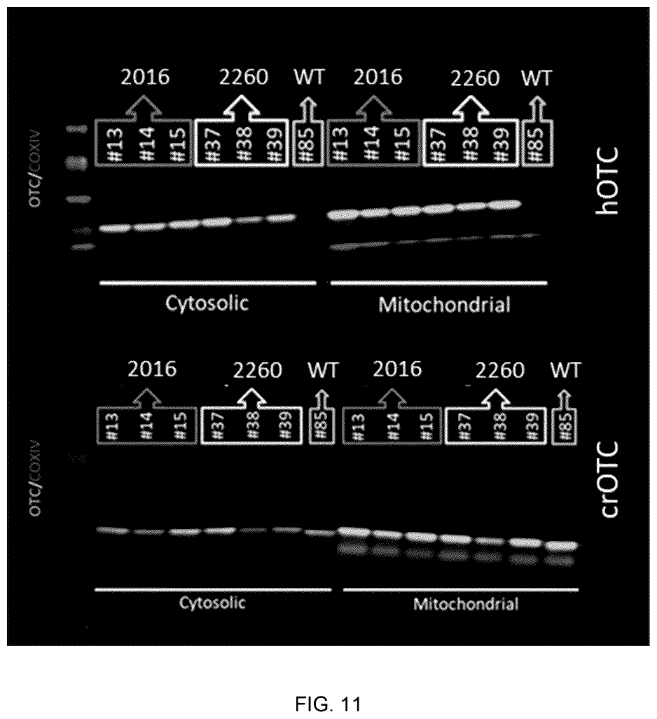

Spf/ash mice received an IV injection with either PBS or lipid-formulated (as described in Example 1) OTC-mRNAs at 1 mg/kg and 3 mg/kg. WT mice were used as internal controls. Sample fractioning was performed on the liver samples, separating a cytosolic and a mitochondrial fraction. OTC levels were measured by WB using human specific (hOTC) and crossreactive (crOTC) antibodies (FIG. 11). Cyclooxygenase IV (CoxIV) was used as a mitochondrial control. OTC protein expression levels were measured by near-infrared fluorescent imaging systems and normalized to total protein. WB indicates differences in OTC expression levels within mitochondrial and cytosolic fractions when 2016 and 2260 mRNAs were delivered in the spf/ash mice. These results indicate that both compounds can efficiently target the mitochondria.

### Example 13: Plot of the Mitochondrial Vs Cytosolic Fractions of Spf/Ash Mice Samples Treated with mRNA Constructs 2016 and 2260

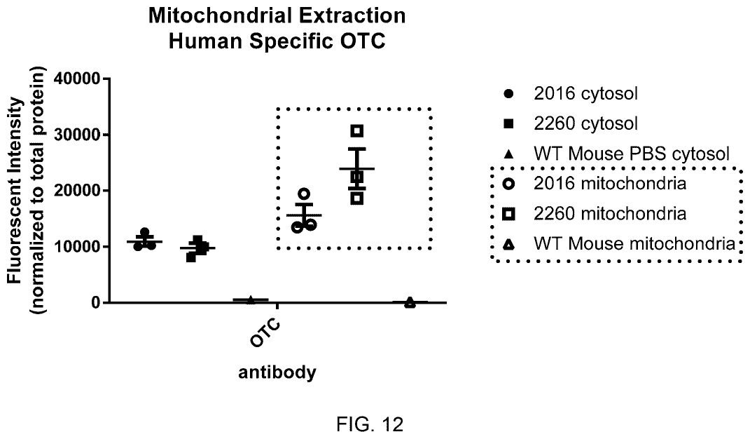

Spf/ash mice received an IV injection with either PBS or lipid-formulated OTC-mRNAs at 3 mg/kg. WT mice were used as internal controls. Sample fractioning was performed on the liver samples, separating a cytosolic and a mitochondrial fraction. OTC levels were measured by western blot using a human specific antibody. OTC protein expression levels were measured by near-infrared fluorescent imaging systems and both fractions normalized to total protein were plotted (FIG. 12). The plot of protein expression levels shown in FIG. 11 (Example 12), indicate that even though both compounds, 2016 and 2260, deliver similar protein levels in the cytosol, it is in the mitochondria where 2260 delivers more human OTC than 2016. The 2260 includes a modified mitochondrial signaling peptide sequence of the invention.

### Example 14: Urinary Orotate Levels in Spf/Ash Mice Treated with mRNA Construct 2260

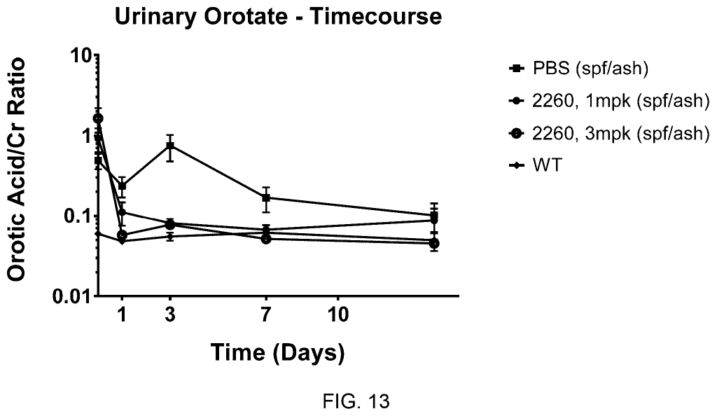

Spf/ash mice received an IV injection with either PBS or lipid-formulated (as described in Example 1) OTC-mRNA at 1 mg/kg and 3 mg/kg. WT mice were included as an internal control. Urinary orotate levels were measured at 0, 1, 3, 7 and 14 days, and levels were normalized to creatinine (FIG. 13). The functional read-out of this assay shows that urinary orotate levels are reduced for up to 14 days with compound 2260, in a dose dependent manner.

### Example 15: Survival of Spf/Ash Mice on a High Protein Diet During Treatment with OTC-mRNA Construct 1799.7 (5MeOU Chemistry)

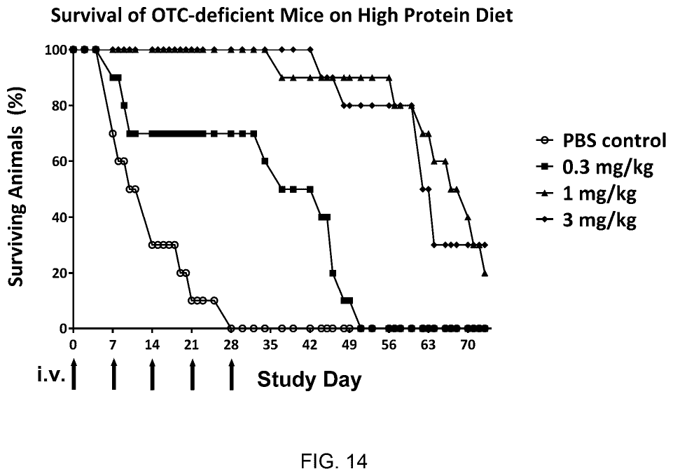

Spf/ash mice received an IV injection with either PBS or lipid-formulated OTC-mRNA (1799.7) at three doses: 0.3 mg/kg, 1 mg/kg and 3 mg/kg. Mice were under a high protein diet since day 0 to the end of the study. Treated animals were injected by IV on days 0, 7, 14, 21 and 28 (arrows). Survival rates were determined every week. The plot in FIG. 14 summarizes the entire study timeline and the survival rates observed for the different groups. The results show that animals treated with human OTC mRNAs described herein have a higher chance to survive during a hyperammonemic crisis, suggesting the protective role of OTC mRNAs described herein in detoxifying the animals from toxic ammonia. This survival rate was dose-dependent, and animals treated with a 3 mg/kg dose have a higher survival rate than animals treated at 1 mg/kg or 0.3 mg/kg.

### Example 16: Comparison of hOTC Expression Levels with Different Modifications

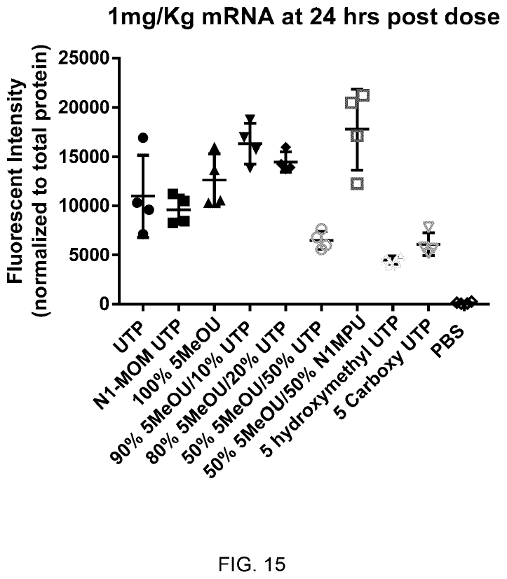

Lipid-formulated OTC-mRNA construct 2262 doses were injected by IV in 8 week-old male C57BL/6 mice at a dose of 1 mg/kg. Different chemistries were used in this study as indicated in the bottom axis of the chart provided in FIG. 15. The mice livers were harvested at 24 hours post IV-administration, and western blot was performed using a hOTC specific antibody. Levels were normalized to total protein (FIG. 15). Each construct was formulated as a lipid nanoparticle comprising an ATX lipid as described in Example 1. This dataset indicates the impact of different uridine chemistries on the expression levels of our codon-optimized mRNAs.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this disclosure has been particularly shown and described with references to preferred embodiments thereof, it will be understood by those skilled in the art that various changes in form and details may be made therein without departing from the scope of the disclosure encompassed by the appended claims. It should also be understood that the embodiments described herein are not mutually exclusive and that features from the various embodiments may be combined in whole or in part in accordance with the disclosure.

1. An ornithine transcarbamylase (OTC) protein comprising the amino acid sequence of SEQ ID NO:4 and having OTC enzymatic activity.

2. A polynucleotide encoding the OTC protein of claim 1.

3. (canceled)

4. (canceled)

5. The polynucleotide of claim 2, wherein the polynucleotide is an mRNA or DNA.

6. The polynucleotide of claim 5, wherein the polynucleotide is an mRNA comprising a 3′poly A tail.

7. (canceled)

8. (canceled)

9. The polynucleotide of claim 5, wherein the polynucleotide is an mRNA comprising a 5′ untranslated region (5′UTR).

10. The polynucleotide of claim 9, wherein the 5′UTR comprises a sequence of a 5′ UTR of human IL-6, alanine aminotransferase 1, human apolipoprotein E, human fibrinogen alpha chain, human transthyretin, human haptoglobin, human alpha-1-antichymotrypsin, human antithrombin, human alpha-1-antitrypsin, human albumin, human beta globin, human complement C3, human complement C5, SynK (thylakoid potassium channel protein derived from the cyanobacteria, Synechocystis sp.), mouse beta globin, mouse albumin, or tobacco etch virus, or a sequence of a 5′ UTR of a gene expressed by Arabidopsis thaliana.

11. The polynucleotide of claim 10, wherein the 5′ UTR comprises a sequence selected from SEQ ID NO: 6, SEQ ID NOS: 125-127 or SEQ ID NOS: 230-250.

12. The polynucleotide of claim 10, wherein the 5′UTR comprises a sequence of a 5′ UTR of mouse beta globin.

13. The polynucleotide of claim 5, wherein the polynucleotide is an mRNA comprising a 3′ UTR.

14. The polynucleotide of claim 13, wherein the 3′ UTR comprises a sequence selected from SEQ ID NOS: 16-22.

15. The polynucleotide of claim 5, wherein the polynucleotide is an mRNA comprising a Kozak sequence of SEQ ID NO: 23 or a partial Kozak sequence of SEQ ID NO: 24.

16. The polynucleotide of claim 5, wherein the polynucleotide is an mRNA comprising a 5′ cap.

17. (canceled)

18. The polynucleotide of claim 5, wherein
(a) the polynucleotide is an mRNA comprising an optimized coding region comprising the sequence of SEQ ID NO: 221, wherein T is substituted with U; or
(b) the polynucleotide is DNA comprising an optimized coding region comprising the sequence of SEQ ID NO: 221.

19. The polynucleotide of claim 5, wherein the polynucleotide is an mRNA comprising the sequence of SEQ ID NO: 119, SEQ ID NO: 251, or SEQ ID NO: 252.

20. The polynucleotide of claim 5, wherein the polynucleotide is an mRNA and wherein
(a) the percentage of uracil nucleobases in the coding region of the polynucleotide is reduced with respect to the percentage of uracil nucleobases in the wild-type OTC nucleic acid sequence; or
(b) 1-100% of the uridines are modified uridine analogs; or
(c) 1-100% of the uridine nucleotides are each independently a modified uridine analog selected from the group consisting of 5-methoxyuridine, N¹-methylpseudouridine, 5-hydroxyuridine, 5-methyluridine, 5-hydroxymethyluridine, 5-carboxyuridine, 5-carboxymethylesteruridine, 5-formyluridine, 5-propynyluridine, 5-bromouridine, 5-fluorouridine, 5-iodouridine, 2-thiouridine, 6-methyluridine, N¹-ethylpseudouridine, N¹-propylpseudouridine, N¹-cyclopropylpseudouridine, N¹-phenylpseudouridine, N¹-aminomethylpseudouridine, N³-methylpseudouridine, N¹-hydroxypseudouridine, N¹-hydroxymethylpseudouridine, 5-methoxycarbonylmethyl-2-thiouridine, 5-methylaminomethyl-2-thiouridine, 5-carbamoylmethyluridine, 5-carbamoylmethyl-2′-O-methyluridine, 1-methyl-3-(3-amino-3-carboxypropyl)pseudouridine, 5-methylaminomethyl-2-selenouridine, 5-carboxymethyluridine, 5-methyldihydrouridine, 5-taurinomethyluridine, 5-taurinomethyl-2-thiouridine, 5-(isopentenylaminomethyl)uridine, 2′-O-methylpseudouridine, 2-thio-2′O-methyluridine, and 3,2′-O-dimethyluridine; or
(d) 100% of the uridine nucleotides are 5-methoxyuridine; or
(e) 100% of the uridine nucleotides are N¹-methylpseudouridine.

21.-27. (canceled)

28. A pharmaceutical composition comprising the polynucleotide of claim 2 and a pharmaceutically acceptable carrier, wherein the pharmaceutically acceptable carried is a lipid formulation.

29. A pharmaceutical composition comprising the polynucleotide of claim 20 and a pharmaceutically acceptable carrier, wherein the pharmaceutically acceptable carried is a lipid formulation.

30.-35. (canceled)

36. A method of treating OTC deficiency in a patient identified as suffering from OTC deficiency, comprising administering to the patient, a pharmaceutical composition of claim 28, wherein upon administration of the pharmaceutical composition to the patient, the OTC protein is expressed in the patient.

37. A method of treating OTC deficiency in a patient identified as suffering from OTC deficiency, comprising administering to the patient a polynucleotide of claim 5, wherein the polynucleotide is an mRNA and expresses the OTC protein in the patient.

38. A vector comprising the polynucleotide of claim 5.

39.-76. (canceled)
